SEQUENCE LISTING (51 sequences)

SEQ ID NO: 1 - DNA, 8667 bp, Zea mays
aataagttgagtcggggtagatctcaaggctacataaaatagttttttttctagaatggatgcatttgttttaagagaaaaatgatgcacttggaatgcatcaagcgaagggatgcaagaatgttggaaaaacacatgaacccgtatcgcgagatgcttatttagccattcttttatcacagtggatgcatatgcaacaaaaccaaaacagatggttagcgagtgacagtatatagagatctaaagattccgacacttcatcggtaaaaaaagcagcattaaccgagtgaatggaagaaaaacgaatttctcatattacacaacaggttttcttaaaaaaaaaaacgttacctcggtatttatattaagaagagactaaaatatggtcccgtcgagaaaatttctaaacactagtcttcatcactagtgagccgtcaccatctagtttgcaacggtccagtcagagtgcaggacataactcaggactcaagagcagacggagcacggtgagagaattttttttaatcaagcctaaaattcacctccggacaaattgaacctggaacgggtgctactcataaatattaaccatgagaccttttcgccgcagcaggttttctatcggcagttagattttagtgacgatgaaaatgatagaacgcaacgtgccgcatgcatccattcccattcgttttccacagtacatgtaggagtactgtgcaagtagggtccgtacattcagtctctctcaccagttggactcttctactgctacaaagacatgagctgcctggaatgggaaccggaggagcgagcgagcctgacggtctcacacacacacagtcacactcccaagccaattattataagaggcgagatgagcaactccagctcttaaccaatccactcctcctccctctccacctcctctgctttgctctgccactctgctgaggtggggggcagaggagctccccctccctcctctcccctcctcgccatgtctagcagcgacccggaggagatcagggcgcgcgtcgtcgttctcggttcgccccatgccgacggcggcgacgagtgggcccggcccgagctcgaggccttccatctgccgtctcccgcccaccagcctcctggcttcctagccgggcaaccggaagcagcagagcaacccacgctccctgctcctgctggccgcagcagcagcagcagcaacacgcctactacatctgccggtggcggcgctgctcctcctcctccttcttcgcctccccctccgccggcttctctggagaccgagcagccgcccaatgccaggccagcctccgccggcgccaatgacagcaagaagcccaccccgcccgccgccctgcgcgacctcttccgcttcgccgacggcctcgactgcgcgctcatgctcatcggcaccctcggcgcgctcgtccacggctgctcgctccccgtcttcctccgcttcttcgccgacctcgtcgactccttcggctcccacgccgacgacccggacaccatggtccgcctcgtcgtcaagtacgccttctacttcctcgtcgtcggagcggcaatctgggcatcctcgtgggcaggtacgctatccctcctcctcctgccgccccagcttgtgtgcgtcgcgaattggcggtcaatttggattggatgacaaatcacgtcggtcagccaatcgccgtggctacaaacgagatgttcaaatcgttcgccccgctcgcagagatctcttgctggatgtggaccggcgagcggcagtcgacgcggatgcggatccggtacctggacgcggcgctgcggcaggacgtgtccttcttcgacaccgacgtgcgggcctcggacgtgatctacgccatcaacgcggacgcagtggtggtgcaggacgccatcagcgagaagctgggcaacctcatccactacatggccaccttcgtggccggcttcgtcgtggggttcacggccgcgtggcagctggcgctggtcacgctggccgtggtgccgctcatcgccgtcatcggcgggctgagcgccgccgcgctcgccaagctctcgtcccgcagccaggacgcgctctccggcgccagcggcatcgcggagcaggcgctcgcgcagatacggatcgtgcaggcgttcgtcggcgaggagcgcgagatgcgggcctactcggcggcgctggccgtggcgcagaggatcggctaccgcagcggcttcgccaagggtctcggcctcggcggcacctacttcaccgtcttctgctgctacgggctcctgctctggtacggcggccacctcgtgcgcgcccagcacaccaacggcgggctcgccatcgccaccatgttctccgtcatgatcggcggactgtaaggcccaccacaccactctctccttctcctgctcctcggcccgcccgtcgtcattgctgctgacggtgtctgtggatcgcgtgcagggccctcgggcagtcggcgccgagcatggccgcgttcgccaaggcgcgtgtggcggctgccaagatcttccgcatcatcgaccacaggccgggcatctcctcgcgcgacggcgcggagccagagtcggtgacggggcgggtggagatgcggggcgtggacttcgcgtacccgtcgcggccggacgtccccatcctgcgcggcttctcgctgagcgtgcccgccgggaagaccatcgcgctggtgggcagctccggctccgggaagagcacggtggtttcgctcatcgagaggttctacgaccccagcgcaggtatactacctagtactgttactacttttagcgcattaatctgaggatgtccagttcgcttgcttgccaatcgccattgccatcgcaacaatacattgccaactgccattgctgggtagactagtactgtagtacagtagcagttagaagaagcctccactgtacattgcattgccaaacaaaagtgaattgtgcagtaactctgtaccaccacattgacatggaaatgaagtgaatgcttggagcatgcagagctggccggcctcatgggctgctgctacctgctagctagccaaccagaaccagccatcctctttcttgcttttctttttactttctttggtcgtggctgtttgtggtcatacatacattcacgcgcagagcagaagagctagctaagctaggtgggtgtgcctgcaacgcgggacaaagaaaactatttgttgcctggcaagatgctactgttgcctagcacatgcctgccattgaccgactgctcagtgagaagtggttcagttgtgctgttgacagtatagatagatatatatatagtagccctgtatattttctttttcagacaagaaaaaaagaagaacgagatgaagtctgcaattcggttttggcagggcaaatcctgctggacgggcacgacctcaggtcgctgaagctgcgatggctgcggcggcagatcgggctggtgagccaggagccggcgctgttcgcgacgagcatcagggagaacctgctgctggggcgggacagccagagcgcgacgctggcggagatggaggaggcggccagggtggccaacgcccactccttcatcatcaaactccccgacggctacgacacgcaggtccgtcccgtatagctagctcactagctgcactgccacttctctcgcttgctccccaccgttgctgcctgttgctctccaatccacttgtcggtgtctggaccacacgtgcctgcttgcctagctgctccacatctgctttccctgtccaaccttatgcaactcactcagtcactctatactatatcaaatacatttctagagtttaaagcttatcttagaataaatgcatctttagctacgagacaacctaacttcagttgttgttgttttttttactttctctcttctcaaaaatacattgattacgtctttacagcgatctttttattccaaacctaaaaatgcatgcactcactctaaaagcgcaaagggagcatcttttttccccatcatctgcacgcagccttttcttttcctcatgtcacgagggactgaaggtgtgtatgcagcgtcaagtcatccatccgttccactccactcactcatgcgtcgcgcactctgcgctcgtgcctgcccggggctaaagctttagtagctagcctcagatcagatactgttcgtgtttgttaggccgcggcagcggcacatgagctcatgacagccggcagcaccaccaccaacaccatggaagaggggtcggggtccatcacatagacatagatgcctgttgtagactagggacgggagggcaattgttaggcgcctgttgccatcgcatttgctgctgtgggttgccaacaagtaacatgccaggatgctttgctatcacgcacaggacaggaggagaggtcctttttctcgacacaagatctactaaactagcacttgctgatgagtgcataggatgaatggatgatgaacatctagagtgagagagagaaaatatgttaataataataaagagtagtaatagtagtagtaggattaagaatcaacctggggtacgtaggaagaggtacaatccctaggaatctagagtatgagaagtatgggaggagttgtgagtgaaacggaacaaattccgagttggtattttgtcgggaatgtcaagttgatttttgatcctagtgcaagcaagaattatcaatcactcagactcagcctgtctgtgtctgtccaccccagctcttgctactctacttactactgtgctactagtggtagggtaggtatcttccataaactgttattataaactgtcatctgagaaagagagccagtcaaacccatgctgctgcttatttttaatcactgtcaaatggcaggcaggcaggcagtctggttagttaataacatctgggaagggtttaatcaaaccaaatcaaatcatacgaaatctagaggccacatgggatggggccatatgtactgtactagcataagtagcggctagattttattagaacacggactcacactcccataactataactgactttgatcatgattccttgccaagcaatgctcgcatgcccatgcatgcatcatccctggtcaaactcaaacactctccaccgtcagggaataagacttattattttattaacaattccatttttatttattaattacggctggacgaggagtactagtttatttgatgagagacatggcagtccaagtcaaactcgtttgtctgaccatggcggtgatggccggttgcaggttggggagcgcggcctgcagctctccggtgggcagaagcagcgcatcgccatcgcccgcgccatgctcaagaaccccgccatcctgctgctggacgaggccaccagcgcgctggactccgagtctgagaagctcgtgcaggaggcgctggaccgcttcatgatcgggcgcaccaccctggtgatcgcgcacaggctgtccaccatccgcaaggccgacgtggtggccgtgctgcagggcggcgccgtctccgagatgggcgcgcacgacgagctgatggccaagggcgagaacggcacctacgccaagctcatccgcatgcaggagcaggcgcacgaggcggcgctcgtcaacgcccgccgcagcagcgccaggccctccagcgcccgcaactccgtcagctcgcccatcatgacgcgcaactcctcctacggccgctccccctactcccgccgcctctccgacttctccacctccgacttcaccctctccatccacgacccgcaccaccaccaccggaccatggcggacaagcagctggcgttccgcgccggcgccagctccttcctgcgcctcgccaggatgaactcgcccgagtgggcctacgcgctcgccggctccatcggctccatggtctgcggctccttcagcgccatcttcgcctacatcctcagcgccgtgctcagcgtctactacgcgccggacccgcggtacatgaagcgcgagatcgcaaaatactgctacctgctcatcggcatgtcctccgcggcgctgctgttcaacacggtgcagcacgtgttctgggacacggtgggcgagaacctgaccaagcgggtgcgcgagaagatgttcgccgccgtgctccgcaacgagatcgcctggttcgacgcggacgagaacgccagcgcgcgcgtggccgccaggctagcgctggacgcccagaacgtgcgctccgccatcggggaccgcatctccgtcatcgtccagaactcggcgctgatgctggtggcctgcaccgcggggttcgtcctccagtggcgcctcgcgctcgtgctcctcgccgtgttcccgctcgtcgtgggcgccaccgtgctgcagaagatgttcatgaagggcttctcgggggacctggaggccgcgcacgccagggcgacgcagatcgcgggcgaggccgtggccaacctgcgcaccgtggccgcgttcaacgcggagcgcaagatcacggggctgttcgaggccaacctgcgcggcccgctccggcgctgcttctggaaggggcagatcgccggcagcggctacggcgtggcgcagttcctgctgtacgcgtcctacgcgctggggctgtggtacgcggcgtggctggtgaagcacggcgtgtccgacttctcgcgcaccatccgcgtgttcatggtgctgatggtgtccgcgaacggcgccgccgagacgctgacgctggcgccggacttcatcaagggcgggcgcgcgatgcggtcggtgttcgagacgatcgaccgcaagacggaggtggagcccgacgacgtggacgcggcgccggtgccggagcggccgaggggcgaggtggagctgaagcacgtggacttctcgtacccgtcgcggccggacatccaggtgttccgcgacctgagcctccgtgcgcgggccgggaagacgctggcgctggtggggccgagcgggtgcggcaagagctcggtgctggcgctggtgcagcggttctacgagcccacgtccgggcgcgtgctcctggacggcaaggacgtgcgcaagtacaacctgcgggcgctgcggcgcgtggtggcggtggtgccgcaggagccgttcctgttcgcggcgagcatccacgagaacatcgcgtacgggcgcgagggcgcgacggaggcggaggtggtggaggcggcggcgcaggcgaacgcgcaccggttcatcgcggcgctgccggaggggtaccggacgcaggtgggcgagcgcggggtgcagctgtcgggagggcagcggcagcggatcgcgatcgcgcgcgcgctggtgaagcaggcggccatcgtgctgctggacgaggcgaccagcgcgctggacgccgagtcggagcggtgcgtgcaggaggcgctggagcgcgcggggtccgggcgcaccaccatcgtggtggcgcaccggctggccacggtgcgcggcgcgcacaccatcgcggtcatcgacgacggcaaggtggcggagcaggggtcgcactcgcacctgctcaagcaccatcccgacgggtgctacgcgcggatgctgcagctgcagcggctgacgggcgcggcggccgggcccgggccgtcgacctcgtgcaacggggccgcgtaggacggaatggatggatggatgggtttggttcctcgagagattgatgggtgaggaagctgaagctccggatcaaatggtggtactccatgatcgcaacaatgaggggaaaaaaagaaaggagaaaatacggtggttcatatgattgtacaatttgacgatctgtttgagtcggggttttaggatgatgtaaaccttcactcgccttttttttactcttgtttctcatccgcatcagtatcatctatctacatacagtgtcagagatgggaactgatccgcatcatcatctacctcccaaggcaccccagattgtattaatgtacttagttagcctgttttatatatacttataagtaccaaatagcagaattttactccttatctgcagtagcacgaaagaagaaacggcaagctacacctatctctggaagcaaaagaatctccccattctctctcttcctacgaggacaccccatccttttcagacagacagacagacggaccaaactgcaccacccaagaaccgatcgagagcagaggtagcattctctgcactgtagagtgtagaccatactacgctgctctcgagacatgatcccgggaatatctaccgaattgtttgtcagtggcatagccgcatagctctcagctgcctaatgaattgtttggtgccaaccgcgctgcaggcttgcagacgacccgggaagggtcagggattgcacaactactagcatcagtggagagcgaaaggccagaacggcaggcttgcaggaaaacagggaccgtgagtttgacctggccttggggtggggagtcagtcagtcacatgcgatgcgatgcagtggaggaacccatcttccaacgcacgcggcacgattgatttcagggttgctttcgccgctgccaatccaccgatccctggacactggtgcgttccttcgtcgcacatgacggtgcgtcgccccaaccgcacaggactgggagaagggacggattgggg

SEQ ID NO: 2 - DNA, 23 bp, Zea mays
gctcatcgagaggttctacgacc

SEQ ID NO: 3 - DNA, 23 bp, Zea mays
atgatgaaggagtgggcgttggc

SEQ ID NO: 4 - DNA, 23 bp, Zea mays
cgatctcgcgcttcatgtaccgc

SEQ ID NO: 5 - DNA, 23 bp, Zea mays
gtgggcagaagcagcgcatcgcc

SEQ ID NO: 6 - DNA, 23 bp, Zea mays
gccgcctctccgacttctccacc

SEQ ID NO: 7 - DNA, 23 bp, Zea mays
cctacatcctcagcgccgtgctc

SEQ ID NO: 8 - DNA, 18 bp, Artificial Sequence
  PCR Primer
ccatcctgcgcggcttct

SEQ ID NO: 9 - DNA, 19 bp, Artificial Sequence
  PCR Primer
atctcgttgcggagcacgg

SEQ ID NO: 10 - DNA, 19 bp, Artificial Sequence
  PCR Primer
gttcgcgacgagcatcagg

SEQ ID NO: 11 - DNA, 40 bp, Zea mays
ggtttcgctcatcgagaggtccaccctggccgcctcctcc

SEQ ID NO: 12 - DNA, 40 bp, Zea mays
ggtagtatacctgcgctggcactccttcatcatcaaactc

SEQ ID NO: 13 - DNA, 40 bp, Zea mays
tggtttcgctcatcgagagggtccggcgcgtagtagacgc

SEQ ID NO: 14 - DNA, 40 bp, Zea mays
tgacattcccgacaaaatactgctacctgctcatcggcat

SEQ ID NO: 15 - DNA, 40 bp, Zea mays
ggtttcgctcatcgagaggtgtccggcgcgtagtagacgc

SEQ ID NO: 16 - DNA, 40 bp, Zea mays
gagtttgatgatgaaggagtccagcgcaggtatactacct

SEQ ID NO: 17 - DNA, 40 bp, Zea mays
tttcgctcatcgagaggttcgccaccctggccgcctcctc

SEQ ID NO: 18 - DNA, 40 bp, Zea mays
tctactacgcgccggacccgatgaagcgcgagatcgcaaa

SEQ ID NO: 19 - DNA, 40 bp, Zea mays
ggtttcgctcatcgagaggttcaccctggccgcctcctcc

SEQ ID NO: 20 - DNA, 40 bp, Zea mays; identical to SEQ ID NO: 12

SEQ ID NO: 21 - DNA, 40 bp, Zea mays; identical to SEQ ID NO: 18

SEQ ID NO: 22 - DNA, 40 bp, Zea mays
tttcgctcatcgagaggttccagcgcaggtatactaccta

SEQ ID NO: 23 - DNA, 40 bp, Zea mays
gaggaggcggccagggtggcactccttcatcatcaaactc

SEQ ID NO: 24 - DNA, 40 bp, Zea mays
tctactacgcgccggacccgaagcgcgagatcgcaaaata

SEQ ID NO: 25 - DNA, 40 bp, Zea mays
gtttcgctcatcgagaggttgtatactacctagtactgtt

SEQ ID NO: 26 - DNA, 40 bp, Zea mays
gagatggaggaggcggccagcactccttcatcatcaaact

SEQ ID NO: 27 - DNA, 40 bp, Zea mays
gtctactacgcgccggaccctgaagcgcgagatcgcaaaa

SEQ ID NO: 28 - DNA, 40 bp, Zea mays
tttcgctcatcgagaggttcgaccccagcgcaggtatact

SEQ ID NO: 29 - DNA, 40 bp, Zea mays
gaggaggcggccagggtggaactccttcatcatcaaactc

SEQ ID NO: 30 - DNA, 40 bp, Zea mays
ggtttcgctcatcgagaggtcgggtccggcgcgtagtaga

SEQ ID NO: 31 - DNA, 40 bp, Zea mays
ggagtttgatgatgaaggaggaagcgcgagatcgcaaaat

SEQ ID NO: 32 - DNA, 40 bp, Zea mays
ttcgctcatcgagaggttctccttcatcatcaaactcccc

SEQ ID NO: 33 - DNA, 40 bp, Zea mays
tacgcgccggacccgcggtaagcgcgagatcgcaaaatac

SEQ ID NO: 34 - DNA, 40 bp, Zea mays
gtttcgctcatcgagaggttccctggccgcctcctccatc

SEQ ID NO: 35 - DNA, 40 bp, Zea mays
ctaggtagtatacctgcgctctccttcatcatcaaactcc

SEQ ID NO: 36 - DNA, 40 bp, Zea mays
ggtttcgctcatcgagaggtatactacctagtactgttac

SEQ ID NO: 37 - DNA, 40 bp, Zea mays; identical to SEQ ID NO: 23

SEQ ID NO: 38 - DNA, 40 bp, Zea mays
ctactacgcgccggacccgcatgaagcgcgagatcgcaaa

SEQ ID NO: 39 - DNA, 877 bp, Zea mays
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttcccc

SEQ ID NO: 40 - DNA, 3750 bp, Lachnospiraceae bacterium G532R/K595R
atggcgggatctaagaagagaagaattaaacaagattcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcacggatctaagaagagaagaattaaacaagattga

SEQ ID NO: 41 - DNA, 30 bp, Solanum lycopersicum
ggatctaagaagagaagaattaaacaagat

SEQ ID NO: 42 - DNA, 200 bp, Zea mays
cggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaatt

SEQ ID NO: 43 - DNA, 19 bp, Artificial Sequence
  RNA assay primer
cagatacggatcgtgcagg

SEQ ID NO: 44 - DNA, 20 bp, Artificial Sequence
  RNA assay primer
gcacctacttcaccgtcttc

SEQ ID NO: 45 - DNA, 19 bp, Artificial Sequence
  RNA assay primer
tggacttctcgtacccgtc

SEQ ID NO: 46 - DNA, 18 bp, Artificial Sequence
  RNA assay primer
ctggtgcagcggttctac

SEQ ID NO: 47 - DNA, 20 bp, Artificial Sequence
  RNA assay probe
tggcgcagaggatcggctac

SEQ ID NO: 48 - DNA, 19 bp, Artificial Sequence
  RNA assay probe
ttccgcgacctgagcctcc

SEQ ID NO: 49 - DNA, 31 bp, Artificial Sequence
  RACE primer
tcgcgcagatacggatcgtgcaggcgttcgt

SEQ ID NO: 50 - DNA, 4548 bp, Zea mays
ggtggggggcagaggagctccccctccctcctctcccctcctcgccatgtctagcagcgacccggaggagatcagggcgcgcgtcgtcgttctcggttcgccccatgccgacggcggcgacgagtgggcccggcccgagctcgaggccttccatctgccgtctcccgcccaccagcctcctggcttcctagccgggcaaccggaagcagcagagcaacccacgctccctgctcctgctggccgcagcagcagcagcagcaacacgcctactacatctgccggtggcggcgctgctcctcctcctccttcttcgcctccccctccgccggcttctctggagaccgagcagccgcccaatgccaggccagcctccgccggcgccaatgacagcaagaagcccaccccgcccgccgccctgcgcgacctcttccgcttcgccgacggcctcgactgcgcgctcatgctcatcggcaccctcggcgcgctcgtccacggctgctcgctccccgtcttcctccgcttcttcgccgacctcgtcgactccttcggctcccacgccgacgacccggacaccatggtccgcctcgtcgtcaagtacgccttctacttcctcgtcgtcggagcggcaatctgggcatcctcgtgggcagagatctcttgctggatgtggaccggcgagcggcagtcgacgcggatgcggatccggtacctggacgcggcgctgcggcaggacgtgtccttcttcgacaccgacgtgcgggcctcggacgtgatctacgccatcaacgcggacgcagtggtggtgcaggacgccatcagcgagaagctgggcaacctcatccactacatggccaccttcgtggccggcttcgtcgtggggttcacggccgcgtggcagctggcgctggtcacgctggccgtggtgccgctcatcgccgtcatcggcgggctgagcgccgccgcgctcgccaagctctcgtcccgcagccaggacgcgctctccggcgccagcggcatcgcggagcaggcgctcgcgcagatacggatcgtgcaggcgttcgtcggcgaggagcgcgagatgcgggcctactcggcggcgctggccgtggcgcagaggatcggctaccgcagcggcttcgccaagggtctcggcctcggcggcacctacttcaccgtcttctgctgctacgggctcctgctctggtacggcggccacctcgtgcgcgcccagcacaccaacggcgggctcgccatcgccaccatgttctccgtcatgatcggcggactggccctcgggcagtcggcgccgagcatggccgcgttcgccaaggcgcgtgtggcggctgccaagatcttccgcatcatcgaccacaggccgggcatctcctcgcgcgacggcgcggagccagagtcggtgacggggcgggtggagatgcggggcgtggacttcgcgtacccgtcgcggccggacgtccccatcctgcgcggcttctcgctgagcgtgcccgccgggaagaccatcgcgctggtgggcagctccggctccgggaagagcacggtggtttcgctcatcgagaggttctacgaccccagcgcagggcaaatcctgctggacgggcacgacctcaggtcgctgaagctgcgatggctgcggcggcagatcgggctggtgagccaggagccggcgctgttcgcgacgagcatcagggagaacctgctgctggggcgggacagccagagcgcgacgctggcggagatggaggaggcggccagggtggccaacgcccactccttcatcatcaaactccccgacggctacgacacgcaggttggggagcgcggcctgcagctctccggtgggcagaagcagcgcatcgccatcgcccgcgccatgctcaagaaccccgccatcctgctgctggacgaggccaccagcgcgctggactccgagtctgagaagctcgtgcaggaggcgctggaccgcttcatgatcgggcgcaccaccctggtgatcgcgcacaggctgtccaccatccgcaaggccgacgtggtggccgtgctgcagggcggcgccgtctccgagatgggcgcgcacgacgagctgatggccaagggcgagaacggcacctacgccaagctcatccgcatgcaggagcaggcgcacgaggcggcgctcgtcaacgcccgccgcagcagcgccaggccctccagcgcccgcaactccgtcagctcgcccatcatgacgcgcaactcctcctacggccgctccccctactcccgccgcctctccgacttctccacctccgacttcaccctctccatccacgacccgcaccaccaccaccggaccatggcggacaagcagctggcgttccgcgccggcgccagctccttcctgcgcctcgccaggatgaactcgcccgagtgggcctacgcgctcgccggctccatcggctccatggtctgcggctccttcagcgccatcttcgcctacatcctcagcgccgtgctcagcgtctactacgcgccggacccgcggtacatgaagcgcgagatcgcaaaatactgctacctgctcatcggcatgtcctccgcggcgctgctgttcaacacggtgcagcacgtgttctgggacacggtgggcgagaacctgaccaagcgggtgcgcgagaagatgttcgccgccgtgctccgcaacgagatcgcctggttcgacgcggacgagaacgccagcgcgcgcgtggccgccaggctagcgctggacgcccagaacgtgcgctccgccatcggggaccgcatctccgtcatcgtccagaactcggcgctgatgctggtggcctgcaccgcggggttcgtcctccagtggcgcctcgcgctcgtgctcctcgccgtgttcccgctcgtcgtgggcgccaccgtgctgcagaagatgttcatgaagggcttctcgggggacctggaggccgcgcacgccagggcgacgcagatcgcgggcgaggccgtggccaacctgcgcaccgtggccgcgttcaacgcggagcgcaagatcacggggctgttcgaggccaacctgcgcggcccgctccggcgctgcttctggaaggggcagatcgccggcagcggctacggcgtggcgcagttcctgctgtacgcgtcctacgcgctggggctgtggtacgcggcgtggctggtgaagcacggcgtgtccgacttctcgcgcaccatccgcgtgttcatggtgctgatggtgtccgcgaacggcgccgccgagacgctgacgctggcgccggacttcatcaagggcgggcgcgcgatgcggtcggtgttcgagacgatcgaccgcaagacggaggtggagcccgacgacgtggacgcggcgccggtgccggagcggccgaggggcgaggtggagctgaagcacgtggacttctcgtacccgtcgcggccggacatccaggtgttccgcgacctgagcctccgtgcgcgggccgggaagacgctggcgctggtggggccgagcgggtgcggcaagagctcggtgctggcgctggtgcagcggttctacgagcccacgtccgggcgcgtgctcctggacggcaaggacgtgcgcaagtacaacctgcgggcgctgcggcgcgtggtggcggtggtgccgcaggagccgttcctgttcgcggcgagcatccacgagaacatcgcgtacgggcgcgagggcgcgacggaggcggaggtggtggaggcggcggcgcaggcgaacgcgcaccggttcatcgcggcgctgccggaggggtaccggacgcaggtgggcgagcgcggggtgcagctgtcgggagggcagcggcagcggatcgcgatcgcgcgcgcgctggtgaagcaggcggccatcgtgctgctggacgaggcgaccagcgcgctggacgccgagtcggagcggtgcgtgcaggaggcgctggagcgcgcggggtccgggcgcaccaccatcgtggtggcgcaccggctggccacggtgcgcggcgcgcacaccatcgcggtcatcgacgacggcaaggtggcggagcaggggtcgcactcgcacctgctcaagcaccatcccgacgggtgctacgcgcggatgctgcagctgcagcggctgacgggcgcggcggccgggcccgggccgtcgacctcgtgcaacggggccgcgtaggacggaatggatggatggatgggtttggttcctcgagagattgatgggtgaggaagctgaagctccggatcaaatggtggtactccatgatcgcaacaatgaggggaaaaaaagaaaggagaaaatacggtggttcatatgattgtacaatttgacgatctgtttgagtcggggttttaggatgatgtaaaccttcactcgccttttttttactcttgtttctcatccgcatcagtatcatctatctacatacagtgtcagagatgggaactgatccgcatcatcatctacctcccaaggcaccccagattgtattaatgtacttagttagcctgttttatatatacttataagtaccaaat

SEQ ID NO: 51 - protein, 1379 aa, Zea mays
MSSSDPEEIRARVVVLGSPHADGGDEWARPELEAFHLPSPAHQPPGFLAGQPEAAEQPTLPAPAGRSSSSSNTPTTSAGGGAAPPPPSSPPPPPASLETEQPPNARPASAGANDSKKPTPPAALRDLFRFADGLDCALMLIGTLGALVHGCSLPVFLRFFADLVDSFGSHADDPDTMVRLVVKYAFYFLVVGAAIWASSWAEISCWMWTGERQSTRMRIRYLDAALRQDVSFFDTDVRASDVIYAINADAVVVQDAISEKLGNLIHYMATFVAGFVVGFTAAWQLALVTLAVVPLIAVIGGLSAAALAKLSSRSQDALSGASGIAEQALAQIRIVQAFVGEEREMRAYSAALAVAQRIGYRSGFAKGLGLGGTYFTVFCCYGLLLWYGGHLVRAQHTNGGLAIATMFSVMIGGLALGQSAPSMAAFAKARVAAAKIFRIIDHRPGISSRDGAEPESVTGRVEMRGVDFAYPSRPDVPILRGFSLSVPAGKTIALVGSSGSGKSTVVSLIERFYDPSAGQILLDGHDLRSLKLRWLRRQIGLVSQEPALFATSIRENLLLGRDSQSATLAEMEEAARVANAHSFIIKLPDGYDTQVGERGLQLSGGQKQRIAIARAMLKNPAILLLDEATSALDSESEKLVQEALDRFMIGRTTLVIAHRLSTIRKADVVAVLQGGAVSEMGAHDELMAKGENGTYAKLIRMQEQAHEAALVNARRSSARPSSARNSVSSPIMTRNSSYGRSPYSRRLSDFSTSDFTLSIHDPHHHHRTMADKQLAFRAGASSFLRLARMNSPEWAYALAGSIGSMVCGSFSAIFAYILSAVLSVYYAPDPRYMKREIAKYCYLLIGMSSAALLFNTVQHVFWDTVGENLTKRVREKMFAAVLRNEIAWFDADENASARVAARLALDAQNVRSAIGDRISVIVQNSALMLVACTAGFVLQWRLALVLLAVFPLVVGATVLQKMFMKGFSGDLEAAHARATQIAGEAVANLRTVAAFNAERKITGLFEANLRGPLRRCFWKGQIAGSGYGVAQFLLYASYALGLWYAAWLVKHGVSDFSRTIRVFMVLMVSANGAAETLTLAPDFIKGGRAMRSVFETIDRKTEVEPDDVDAAPVPERPRGEVELKHVDFSYPSRPDIQVFRDLSLRARAGKTLALVGPSGCGKSSVLALVQRFYEPTSGRVLLDGKDVRKYNLRALRRVVAVVPQEPFLFAASIHENIAYGREGATEAEVVEAAAQANAHRFIAALPEGYRTQVGERGVQLSGGQRQRIAIARALVKQAAIVLLDEATSALDAESERCVQEALERAGSGRTTIVVAHRLATVRGAHTIAVIDDGKVAEQGSHSHLLKHHPDGCYARMLQLQRLTGAAAGPGPSTSCNGAA

(19) United States
(12) Patent Application Publication
(10) Pub. No.: US 2023/0416770 A1
(43) Pub. Date: Dec. 28, 2023

(54) Methods and Compositions for Generating Dominant Brachytic Alleles Using Genome Editing

(71) Applicant: Monsanto Technology LLC, St. Louis, MO (US)

(72) Inventors: Edward J. CARGILL, Chesterfield, MO (US); Linda A. RYMARQUIS, High Ridge, MO (US); Thomas L. SLEWINSKI, Chesterfield, MO (US); Michelle VALENTINE, Foristell, MO (US)

(73) Assignee: Monsanto Technology LLC, St. Louis, MO (US)

(21) Appl. No.: 18/253,652

(22) PCT Filed: Nov. 19, 2021
(86) PCT No.: PCT/US2021/060116

§ 371 Date: May 19, 2023

Related U.S. Application Data
(60) Provisional application No. 63/117,225, filed on Nov. 23, 2020.

(51) Int. Cl. C12N 15/82; A01H 6/46

(52) U.S. Cl. CPC C12N 15/8262; C12N 15/8218; A01H 6/4684

(57) ABSTRACT

The present disclosure provides compositions and methods for altering auxin accumulation in corn or other cereal plants. Methods and compositions are also provided for altering the expression of genes related to auxin efflux through editing of a Brachytic2 (br2) gene to introduce an antisense sequence or segment or a deletion or premature stop codon into the gene. Modified plant cells and plants having a dominant or semi-dominant allele reducing the expression or activity of a br2 gene product are further provided comprising improved characteristics, such as reduced plant height and increased lodging resistance, but without off-types in the plant.

### CROSS-REFERENCE TO RELATED APPLICATIONS

This application claims benefit of U.S. Provisional Application No. 63/117,225, filed Nov. 23, 2020, which is incorporated by reference in its entirety herein.

### INCORPORATION OF SEQUENCE LISTING

The instant application contains a Sequence Listing which has been submitted electronically in ASCII format and is hereby incorporated by reference in its entirety. Said ASCII copy, created on Nov. 19, 2021, is named P34821WO00_SL.txt and is 43,155 bytes in size.

### FIELD

The present disclosure relates to dominant or semi-dominant alleles of the brachytic 2 gene generated via targeted genome editing.

### BACKGROUND

Sustained increases in crop yields have been achieved over the past few decades last century through the development of improved varieties and agronomic practices. Semi-dwarf varieties of certain crops, such as wheat and rice, were developed having reduced plant height and improved lodging tolerance have been developed. Moreover, dwarf and semi-dwarf traits or varieties have the potential for higher planting densities to help improve crop yields. Indeed, the development of dwarf and semi-dwarf varieties of wheat and rice served as a cornerstone of the so-called “Green revolution” of the late 20th century.

Maize (Zea mays L.), a member of the Poaceae (or Gramineae) Family, provides cylindrical stalks similar to those from other grasses. Commercial hybrid maize can grow to a height of more than 2 meters with each plant having either one or two ears. As a result of its height and vertical structure, a maize plant can be subjected to significant mechanical forces, particularly during high-wind weather events, that can cause maize plants to lodge resulting in a loss of harvestable yield. Conversely, a reduction in the height of a maize plant can improve its mechanical stability and lodging resistance under such conditions.

Many dwarfing mutants have been described in maize, but a majority of these mutants lead to reductions in grain yield and consequently have not been used to enhance crop yield in corn despite the potential lodging benefit. Therefore, an important goal in commercial breeding is to identify novel dwarf or semi-dwarf mutations that confer a short stature phenotype without negatively impacting other organs, especially reproductive organs (e.g., ears), that could ultimately impact yield. In maize, brachytic mutants have been shown to have a short stature phenotype due to shortening of internode lengths without a corresponding reduction in the number of internodes or the number and size of other organs, including the leaves, ear and tassel.

Three brachytic mutants have been isolated in maize to date: brachytic1 (br1), brachytic2 (br2) and brachytic3 (br3). Br3 is also commonly referred to as brevis plant 1 (bv1). Both br1 and br3 mutations cause a reduction in corn plant height which has been thought too severe for commercial exploitation due to potential impacts over yield. In contrast, br2 mutants have particular agronomic potential because of the shortening of the lower stalk internodes with no obvious reduction in other plant organs. In addition, br2 lines exhibit an increased stalk strength and tolerance to wind lodging, while the leaves are often darker and persist longer as active green leaves than corresponding wild-type plants. See, e.g., PCT/US2016/029492, the entire content and disclosure of which are incorporated herein by reference.

There is a need for the development of dominant or semi-dominant traits that cause a dwarf or semi-dwarf phenotype in corn or maize plants that can be used to improve yield and/or lodging resistance and which do not need to be present in a homozygous state to provide a yield and/or lodging benefit, thus facilitating the production of hybrid corn plants carrying the trait. The present disclosure provides dominant or semi-dominant mutations or edits of the endogenous br2 locus that can produce a dwarf or semi-dwarf trait with improved yield and/or lodging resistance in corn or maize plants.

### SUMMARY

In an aspect, this disclosure provides a modified corn plant, or plant part thereof, comprising a mutant allele at an endogenous br2 locus, where the mutant allele comprises a DNA insertion encoding an antisense RNA sequence complementary to at least a portion of a br2 mRNA sequence, wherein the mutant allele produces a RNA transcript comprising the antisense RNA sequence and is able to suppress the expression of a wild-type allele of the br2 locus.

In an aspect, the present disclosure provides a modified corn plant, or plant part thereof, comprising a mutant allele of the endogenous br2 locus, where the mutant allele comprises a DNA segment inserted into the endogenous br2 locus, where the DNA segment encodes an antisense RNA sequence that is at least 70%, at least 75%, at least 80%, at least 85%, at least 90%, at least 95%, at least 96%, at least 97%, at least 98%, at least 99%, or 100% complementary to at least 20, at least 25, at least 30, at least 40, at least 50, at least 75, at least 100, at least 150, at least 200, at least 300, at least 400, at least 500, at least 750, or at least 1000 consecutive nucleotides of one or more of SEQ ID NOs: 1 and 50, and where the mutant allele produces a RNA transcript comprising the antisense RNA sequence.

In an aspect, the present disclosure provides a method for producing a mutant allele of the endogenous br2 locus, the method comprising: (a) generating a first double-stranded break (DSB) in the endogenous br2 locus in a corn cell using a targeted editing technique; and (b) inserting at the first DSB a DNA segment using a targeted editing technique, where the DNA segment encodes an antisense RNA sequence that is at least 70%, at least 75%, at least 80%, at least 85%, at least 90%, at least 95%, at least 96%, at least 97%, at least 98%, at least 99%, or 100% complementary to at least 20, at least 25, at least 30, at least 40, at least 50, at least 75, at least 100, at least 150, at least 200, at least 300, at least 400, at least 500, at least 750, or at least 1000 consecutive nucleotides of one or more of SEQ ID NOs: 1 and 50, and where the mutant allele of the endogenous br2 locus produces a RNA transcript comprising the antisense RNA sequence.

In an aspect, this disclosure provides a method for generating a corn plant comprising: (a) fertilizing at least one female corn plant with pollen from a male corn plant, where the female corn plant comprises a mutant allele of an endogenous Brachytic2 (br2) locus, where the mutant allele comprises a DNA segment inserted into the endogenous br2 locus, where the DNA segment encodes an antisense RNA sequence that is at least 70%, at least 75%, at least 80%, at least 85%, at least 90%, at least 95%, at least 96%, at least 97%, at least 98%, at least 99%, or 100% complementary to at least 20, at least 25, at least 30, at least 40, at least 50, at least 75, at least 100, at least 150, at least 200, at least 300, at least 400, at least 500, at least 750, or at least 1000 consecutive nucleotides of one or more of SEQ ID NOs: 1 and 50, and where the mutant allele of the endogenous br2 locus produces a RNA transcript comprising the antisense RNA sequence; and (b) obtaining at least one seed produced by said fertilizing of step (a). In another aspect, the method further comprises (c) growing the at least one seed obtained in step (b) to generate at least one progeny corn plant comprising said mutant allele. In an aspect, the at least one progeny corn plant obtained in step (c) is heterozygous for the mutant allele.

In an aspect, this disclosure provides a modified corn plant, or plant part thereof, comprising a deletion within an endogenous br2 locus as compared to a control corn plant or plant part thereof.

In an aspect, this disclosure provides a modified corn plant, or plant part thereof, comprising a deletion of or within at least one exon of an endogenous br2 locus as compared to a control corn plant or plant part thereof.

In an aspect, this disclosure provides a modified corn plant, or plant part thereof, comprising a deletion of at least one nucleotide from at least one exon of an endogenous br2 locus as compared to a control corn plant or plant part thereof.

In an aspect, this disclosure provides a modified corn plant, or plant part thereof, comprising a premature stop codon within a nucleic acid sequence encoding a Brachytic2 protein as compared to a nucleic acid sequence of a control corn plant or plant part thereof.

In an aspect, this disclosure provides a method for producing a mutant allele of an endogenous Brachytic2 (br2) locus, the method comprising (a) generating at least a first double-stranded break (DSB) and a second DSB in the endogenous br2 locus in at least one corn cell using a targeted editing technique; and (b) identifying at least one corn cell from step (a) comprising a deletion of the endogenous br2 locus between the first DSB and the second DSB.

In an aspect, this disclosure provides a method for producing a mutant allele of an endogenous Brachytic2 (br2) locus, the method comprising: (a) generating at least a double-stranded break (DSB) in the endogenous br2 locus in at least one corn cell using a targeted editing technique; and (b) identifying at least one corn cell, corn seed or corn plant from the at least one corn cell in step (a) comprising a premature stop codon in the coding sequence of the endogenous br2 locus.

### BRIEF DESCRIPTION OF THE DRAWINGS

FIG. 1 comprises FIG. 1A and FIG. 1B, which comprise schematics of br2 editing strategies. FIG. 1A depicts a strategy for generating inversions or antisense sequences in a br2 gene, and FIG. 1B depicts a strategy for generating hairpins in a br2 gene.

FIG. 2 comprises FIG. 2A, FIG. 2B, and FIG. 2C, which depict three different edited alleles of the br2 locus or gene relative to the wild-type (WT) gene and target sites of the guide RNAs.

FIG. 3 comprises FIG. 3A and FIG. 3B, which depict RNA expression of br2 in F2 edited plants. FIGS. 3A and 3B depict RNA expression levels of br2 at the base of the third leaf or the sixth node, respectively, in wildtype (WT) plants, homozygous (HOM) plants for the edited allele, heterozygous (HET) plants for the edited allele, and null segregants.

FIG. 4 depicts RNA expression in F4 edited plants in leaf, node, and internode tissue.

FIG. 5 comprises FIG. 5A, FIG. 5B, FIG. 5C, FIG. 5D, and FIG. 5E, which each characterize br2 transcripts via a quantitative RACE assay for wildtype (FIG. 5A); edit 1 (FIG. 5B); edit 2 (FIG. 5C); edit 3 (FIG. 5D); and edit 4 (FIG. 5E).

### DETAILED DESCRIPTION

Unless defined otherwise herein, terms are to be understood according to conventional usage by those of ordinary skill in the relevant art. To facilitate understanding of the disclosure, several terms and abbreviations as used herein are defined below as follows:

The term “and/or” when used in a list of two or more items, means that any one of the listed items can be employed by itself or in combination with any one or more of the listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

The term “about” as used herein, is intended to qualify the numerical values that it modifies, denoting such a value as variable within a margin of error. When no particular margin of error, such as a standard deviation to a mean value, is recited, the term “about” should be understood to mean that range which would encompass the recited value and the range which would be included by rounding up or down to that figure, taking into account significant figures.

As used herein, a “plant” includes an explant, plant part, seedling, plantlet or whole plant at any stage of regeneration or development. The term “cereal plant” as used herein refers a monocotyledonous (monocot) crop plant that is in the Poaceae or Gramineae family of grasses and is typically harvested for its seed, including, for example, wheat, corn, rice, millet, barley, sorghum, oat and rye. As commonly understood, a “corn plant” or “maize plant” refers to any plant of species Zea mays and includes all plant varieties that can be bred with corn, including wild maize species.

As used herein, a “plant part” can refer to any organ or intact tissue of a plant, such as a meristem, shoot organ/structure (e.g., leaf, stem or node), root, flower or floral organ/structure (e.g., bract, sepal, petal, stamen, carpel, anther and ovule), seed (e.g., embryo, endosperm, and seed coat), fruit (e.g., the mature ovary), propagule, or other plant tissues (e.g., vascular tissue, dermal tissue, ground tissue, and the like), or any portion thereof. Plant parts of the present disclosure can be viable, nonviable, regenerable, and/or non-regenerable. A “propagule” can include any plant part that can grow into an entire plant.

As used herein, “locus” is a chromosomal locus or region where a polymorphic nucleic acid, trait determinant, gene, or marker is located. A “locus” can be shared by two homologous chromosomes to refer to their corresponding locus or region.

As used herein, “allele” refers to an alternative nucleic acid sequence of a gene or at a particular locus (e.g., a nucleic acid sequence of a gene or locus that is different than other alleles for the same gene or locus). Such an allele can be considered (i) wild-type or (ii) mutant if one or more mutations or edits are present in the nucleic acid sequence of the mutant allele relative to the wild-type allele. Thus, a “mutant allele” of an endogenous gene or locus is an allele of the gene or locus comprising one or more edit(s) and/or mutation(s). If a mutant allele comprises one or more edits, then the mutant allele can also be referred to as an “edited allele.” A mutant allele for a gene may have a reduced or eliminated activity or expression level for the gene relative to the wild-type allele. For diploid organisms such as corn, a first allele can occur on one chromosome, and a second allele can occur at the same locus on a second homologous chromosome. If one allele at a locus on one chromosome of a plant is a mutant allele and the other corresponding allele on the homologous chromosome of the plant is wild-type, then the plant is described as being heterozygous for the mutant allele. However, if both alleles at a locus are mutant alleles, then the plant is described as being homozygous for the mutant alleles. A plant homozygous for mutant alleles at a locus may comprise the same mutant allele or different mutant alleles if heteroallelic or biallelic.

As used herein, an “endogenous locus” refers to a locus at its natural and original chromosomal location. As used herein, the “endogenous br2 locus” refers to the brachytic2 (br2) genic locus at its original chromosomal or genomic location in a corn or maize plant.

As used herein, a “gene” refers to a nucleic acid sequence forming a genetic and functional unit and coding for one or more sequence-related RNA and/or polypeptide molecules. A gene generally contains a coding region operably linked to appropriate regulatory sequences that regulate the expression of a gene product (e.g., a polypeptide or a functional RNA). A gene can have various sequence elements, including, but not limited to, a promoter, an untranslated region (UTR), exons, introns, and other upstream or downstream regulatory sequences.

As used herein, in the context of a protein-coding gene, an “exon” refers to a segment of a DNA or RNA molecule containing information coding for a protein or polypeptide sequence.

As used herein, an “intron” refers to a segment of a DNA or RNA molecule, which does not contain information coding for a protein or polypeptide, and which is first transcribed into a RNA sequence but then spliced out from a mature RNA molecule.

As used herein, an “untranslated region (UTR)” refers to a segment of a RNA molecule or sequence (e.g., a mRNA molecule) expressed from a gene (or transgene), but excluding the exon and intron sequences of the RNA molecule. An “untranslated region (UTR)” also refers a DNA segment or sequence encoding such a UTR segment of a RNA molecule. An untranslated region can be a 5′-UTR or a 3′-UTR depending on whether it is located at the 5′ or 3′ end of a DNA or RNA molecule or sequence relative to a coding region of the DNA or RNA molecule or sequence (i.e., upstream or downstream of the exon and intron sequences, respectively).

As used herein, the term “expression” refers to the biosynthesis of a gene product, and typically the transcription and/or translation of a nucleotide sequence, such as an endogenous gene, a heterologous gene, a transgene or a RNA and/or protein coding sequence, in a cell, tissue, organ, or organism, such as a plant, plant part or plant cell, tissue or organ.

As used herein, a “stem-loop structure” refers to a secondary structure in a RNA molecule having a double stranded region (e.g., stem) made up by two annealing RNA strands, sequences or segments, connected by a single stranded intervening RNA sequence (e.g., a loop or hairpin). A “stem-loop structure” can have a more complicated secondary RNA structure, for example, comprising self-annealing double stranded RNA sequences having internal mismatches, bulges and/or loops.

As used herein, a “native sequence” refers to a nucleic acid sequence naturally present in its original chromosomal location.

As used herein, a “wild-type gene” or “wild-type allele” refers to a gene or allele having a sequence or genotype that is most common in a particular plant species, or another sequence or genotype with natural variations, polymorphisms, or other silent mutations relative to the most common sequence or genotype that do not significantly impact the expression and activity of the gene or allele. Indeed, a “wild-type” gene or allele contains no variation, polymorphism, or any other type of mutation that substantially affects the normal function, activity, expression, or phenotypic consequence of the gene or allele.

The terms “percent identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. For purposes of calculating “percent identity” between DNA and RNA sequences, a uracil (U) of a RNA sequence is considered identical to a thymine (T) of a DNA sequence. If the window of comparison is defined as a region of alignment between two or more sequences (i.e., excluding nucleotides at the 5′ and 3′ ends of aligned polynucleotide sequences, or amino acids at the N-terminus and C-terminus of aligned protein sequences, that are not identical between the compared sequences), then the “percent identity” may also be referred to as a “percent alignment identity”. If the “percent identity” is being calculated in relation to a reference sequence without a particular comparison window being specified, then the percent identity is determined by dividing the number of matched positions over the region of alignment by the total length of the reference sequence. Accordingly, for purposes of the present disclosure, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

For optimal alignment of sequences to calculate their percent identity, various pair-wise or multiple sequence alignment algorithms and programs are known in the art, such as ClustalW, or Basic Local Alignment Search Tool® (BLAST®), etc., that may be used to compare the sequence identity or similarity between two or more nucleotide or protein sequences. Although other alignment and comparison methods are known in the art, the alignment between two sequences (including the percent identity ranges described above) may be as determined by the ClustalW or BLAST® algorithm, see, e.g., Chenna R. et al., “Multiple sequence alignment with the Clustal series of programs,” Nucleic Acids Research 31: 3497-3500 (2003); Thompson J D et al., “Clustal W: Improving the sensitivity of progressive multiple sequence alignment through sequence weighting, position-specific gap penalties and weight matrix choice,” Nucleic Acids Research 22: 4673-4680 (1994); and Larkin M A et al., “Clustal W and Clustal X version 2.0,” Bioinformatics 21: 2947-48 (2007); and Altschul, S. F., Gish, W., Miller, W., Myers, E. W. & Lipman, D. J. (1990) “Basic local alignment search tool.” J. Mol. Biol. 215:403-410 (1990), the entire contents and disclosures of which are incorporated herein by reference.

The terms “percent complementarity” or “percent complementary”, as used herein in reference to two nucleotide sequences, is similar to the concept of percent identity but refers to the percentage of nucleotides of a query sequence that optimally base-pair or hybridize to nucleotides of a subject sequence when the query and subject sequences are linearly arranged and optimally base paired without secondary folding structures, such as loops, stems or hairpins. Such a percent complementarity may be between two DNA strands, two RNA strands, or a DNA strand and a RNA strand. The “percent complementarity” is calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences may be determined based on the known pairings of nucleotide bases, such as G-C, A-T, and A-U, through hydrogen bonding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present disclosure, when two sequences (query and subject) are optimally base-paired (with allowance for mismatches or non-base-paired nucleotides but without folding or secondary structures), the “percent complementarity” for the query sequence is equal to the number of base-paired positions between the two sequences divided by the total number of positions in the query sequence over its length (or by the number of positions in the query sequence over a comparison window), which is then multiplied by 100%.

As used herein, with respective to a given sequence, a “complement”, a “complementary sequence” and a “reverse complement” are used interchangeably. All three terms refer to the inversely complementary sequence of a nucleotide sequence, i.e. to a sequence complementary to a given sequence in reverse order of the nucleotides. As an example, the reverse complement of a nucleotide sequence having the sequence 5′-atggttc-3′ is 5′-gaaccat-3′.

As used herein, the term “antisense” refers to DNA or RNA sequences that are complementary to a specific DNA or RNA sequence. Antisense RNA molecules are single-stranded nucleic acids which can combine with a sense RNA strand or sequence or mRNA to form duplexes due to complementarity of the sequences. The term “antisense strand” refers to a nucleic acid strand that is complementary to the “sense” strand. The “sense strand” of a gene or locus is the strand of DNA or RNA that has the same sequence as a RNA molecule transcribed from the gene or locus (with the exception of Uracil in RNA and Thymine in DNA).

As used herein, an “inverted genomic fragment” refers to a genomic segment that is inverted in the genome such that the original sense strand and antisense strand sequences are reversed or switched in the opposite orientation for the entire genomic segment.

As used herein, in the context of a “corresponding endogenous sequence” or a “corresponding endogenous DNA segment,” an endogenous sequence or endogenous DNA segment is considered to correspond to another sequence or DNA segment (e.g., an non-endogenous, introduced or inserted sequence or DNA segment) when the sequences or DNA segments share sufficient sequence homology, identity, or complementarity.

As used herein, unless specified otherwise, the relative location of two sequence elements of a genic locus, when expressed as “upstream,” “downstream,” “at the 5′ end,” or “at the 3′ end,” is determined based on the direction of the transcription activity associated with that genic locus. For example, for two transcribed genomic DNA elements, their relative location is based on their sense strand where the first genomic DNA element is upstream or at the 5′ end of the second genomic DNA element when the first genomic DNA element is transcribed first.

The term “operably linked” refers to a functional linkage between a promoter or other regulatory element and an associated transcribable DNA sequence or coding sequence of a gene (or transgene), such that the promoter, etc., operates or functions to initiate, assist, affect, cause, and/or promote the transcription and expression of the associated transcribable DNA sequence or coding sequence, at least in certain cell(s), tissue(s), developmental stage(s), and/or condition(s). Two transcribable DNA sequences can also be “operably linked” to each other if their transcription is subject to the control of a common promoter or other regulatory element.

As used herein, an “encoding region” or “coding region” refers to a portion of a polynucleotide that encodes a functional unit or molecule (e.g., without being limiting, a mRNA, protein, or non-coding RNA sequence or molecule). An “encoding region” or “coding region” can contain, for example, one or more exons, one or more introns, a 5′-UTR, a 3′-UTR, or any combination thereof.

As used herein, “adjacent” refers to a nucleic acid sequence that is in close proximity, or next to another nucleic acid sequence. In one aspect, adjacent nucleic acid sequences are physically linked. In another aspect, adjacent nucleic acid sequences or genes are immediately next to each other such that there are no intervening nucleotides between the end of a first nucleic acid sequence and the start of a second nucleic acid sequence. In an aspect, a first gene and a second gene are adjacent to each other if they are separated by less than 50,000, less than 25,000, less than 10,000, less than 9000, less than 8000, less than 7000, less than 6000, less than 5000, less than 4000, less than 3000, less than 2500, less than 2000, less than 1750, less than 1500, less than 1250, less than 1000, less than 900, less than 800, less than 700, less than 600, less than 500, less than 400, less than 300, less than 200, less than 100, less than 75, less than 50, less than 25, less than 20, less than 10, less than 5, less than 4, less than 3, less than 2, or less than 1 nucleotide.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system), a transcription activator-like effector (TALE) nuclease (TALEN), a recombinase, or a transposase. As used herein, “editing” or “genome editing” refers to generating a targeted mutation, deletion, inversion or substitution of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous plant genome nucleic acid sequence. As used herein, “editing” or “genome editing” also encompasses the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a plant. An “edit” or “genomic edit” in the singular refers to one such targeted mutation, deletion, inversion, substitution and/or insertion, whereas “edits” or “genomic edits” refers to two or more targeted mutation(s), deletion(s), inversion(s), substitution(s) and/or insertion(s), with each “edit” being introduced via a targeted genome editing technique. In an aspect, an edit can comprise a deletion and an inversion.

As used herein, “modified” in the context of a plant, plant seed, plant part, plant cell, and/or plant genome, refers to a plant, plant seed, plant part, plant cell, and/or plant genome comprising an engineered change in one or more genes of interest relative to a wild-type or control plant, plant seed, plant part, plant cell, and/or plant genome that alters the expression level and/or coding sequence of the one or more genes of interest. The term “modified” may further refer to a plant, plant seed, plant part, plant cell, and/or plant genome having one or more inversions, deletions, insertions, or combinations thereof, affecting expression of an endogenous br2 gene, or function of an endogenous Br2 protein (encoded by a br2 gene or allele), introduced through chemical mutagenesis, radiation mutagenesis, transposon insertion or excision, or any other known mutagenesis technique, or introduced through genome editing. For clarity, therefore, a modified plant, plant seed, plant part, plant cell, and/or plant genome includes a mutated and/or edited plant, plant seed, plant part, plant cell, and/or plant genome having a modified expression level, expression pattern, and/or coding sequence of a br2 gene and/or Br2 protein relative to a wild-type or control plant, plant seed, plant part, plant cell, and/or plant genome. Modified plants can be homozygous or heterozygous for any given mutation or edit, and/or may be biallelic or heteroallelic for one or more mutations and/or edits at a br2 gene locus. A modified plant is bi-allelic or heteroallelic for a br2 gene if each copy of the br2 gene is a different allele (i.e., comprises different mutation(s) and/or edit(s)), wherein each allele modifies the expression level and/or activity of the br2 gene and/or Br2 protein. Modified plants, plant parts, seeds, etc., may have been subjected to mutagenesis, genome editing or site-directed integration (e.g., without being limiting, via methods using site-specific nucleases), genetic transformation (e.g., without being limiting, via methods of Agrobacterium transformation or microprojectile bombardment), or a combination thereof. Such “modified” plants, plant seeds, plant parts, and plant cells include plants, plant seeds, plant parts, and plant cells that are offspring or derived from “modified” plants, plant seeds, plant parts, and plant cells that retain the molecular change (e.g., change in expression level and/or activity) to the br2 gene. A modified seed provided herein may give rise to a modified plant provided herein. A modified plant, plant seed, plant part, plant cell, or plant genome provided herein may comprise a mutation or edit of a br2 gene as provided herein. A “modified plant product” may be any product made from a modified plant, plant part, plant cell, or plant chromosome provided herein, or any portion or component thereof.

As used herein, the term “control plant” (or likewise a “control” plant seed, plant part, plant cell and/or plant genome) refers to a plant (or plant seed, plant part, plant cell and/or plant genome) that is used for comparison to a modified plant (or modified plant seed, plant part, plant cell and/or plant genome) and has the same or similar genetic background (e.g., same parental lines, hybrid cross, inbred line, testers, etc.) as the modified plant (or plant seed, plant part, plant cell and/or plant genome), except for a mutation(s) and/or genome edit(s) (e.g., inversion, deletion, antisense insertion) in or affecting a br2 gene. For example, a control plant may be an inbred line that is the same as the inbred line used to make the modified plant, or a control plant may be the product of the same hybrid cross of inbred parental lines as the modified plant, except for the absence in the control plant of any mutation(s) or genome edit(s) in or affecting a br2 gene. Similarly, an unmodified control plant refers to a plant that shares a substantially similar or essentially identical genetic background as a modified plant, but without the one or more engineered changes to the genome (e.g., transgene, mutation or edit) of the modified plant. For purposes of comparison to a modified plant, plant seed, plant part, plant cell and/or plant genome, a “wild-type plant” (or likewise a “wild-type” plant seed, plant part, plant cell and/or plant genome) refers to a non-transgenic and non-genome edited control plant, plant seed, plant part, plant cell and/or plant genome. As used herein, a “control” plant, plant seed, plant part, plant cell and/or plant genome may also be a plant, plant seed, plant part, plant cell and/or plant genome having a similar (but not the same or identical) genetic background to a modified plant, plant seed, plant part, plant cell and/or plant genome, if deemed sufficiently similar for comparison of the characteristics or traits to be analyzed.

As used herein, a “target site” for genome editing refers to the location of a polynucleotide sequence within a plant genome that is bound and cleaved by a site-specific nuclease introducing a double stranded break (or single-stranded nick) into the nucleic acid backbone of the polynucleotide sequence and/or its complementary DNA strand. A target site may comprise at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for a RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further below). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a plant genome that is bound and cleaved by another site-specific nuclease that may not be guided by a non-coding RNA molecule, such as a meganuclease, zinc finger nuclease (ZFN), or a TALEN, to introduce a double stranded break (or single-stranded nick) into the polynucleotide sequence and/or its complementary DNA strand. As used herein, a “target region” or a “targeted region” refers to a polynucleotide sequence or region that is flanked by two or more target sites. Without being limiting, in some embodiments a target region may be subjected to a mutation, deletion, insertion or inversion. The term “flanked” when used to describe a target region of a polynucleotide sequence or molecule, refers to two or more target sites of the polynucleotide sequence or molecule surrounding the target region, with one target site on each side of the target region.

As used herein, a “donor template”, which may be a recombinant DNA donor template, is defined as a nucleic acid molecule having a nucleic acid template or insertion sequence for site-directed, targeted insertion or recombination into the genome of a plant cell via repair of a nick or double-stranded DNA break in the genome of a plant cell. For example, a “donor template” may be used for site-directed integration of a DNA segment encoding an antisense sequence of interest, or as a template to introduce a mutation, such as an insertion, deletion, etc., into a target site within the genome of a plant. In an aspect, a donor template introduces a premature stop codon into a target site within the genome of a plant. A targeted genome editing technique provided herein may comprise the use of one or more, two or more, three or more, four or more, or five or more donor templates. A “donor template” may be a single-stranded or double-stranded DNA or RNA molecule or plasmid. An “insertion sequence” of a donor template is a sequence designed for targeted insertion into the genome of a plant cell, which may be of any suitable length. For example, the insertion sequence of a donor template may be between 2 and 50,000, between 2 and 10,000, between 2 and 5000, between 2 and 1000, between 2 and 500, between 2 and 250, between 2 and 100, between 2 and 50, between 2 and 30, between 15 and 50, between 15 and 100, between 15 and 500, between 15 and 1000, between 15 and 5000, between 18 and 30, between 18 and 26, between 20 and 26, between 20 and 50, between 20 and 100, between 20 and 250, between 20 and 500, between 20 and 1000, between 20 and 5000, between 20 and 10,000, between 50 and 250, between 50 and 500, between 50 and 1000, between 50 and 5000, between 50 and 10,000, between 100 and 250, between 100 and 500, between 100 and 1000, between 100 and 5000, between 100 and 10,000, between 250 and 500, between 250 and 1000, between 250 and 5000, or between 250 and 10,000 nucleotides or base pairs in length. A donor template may also have at least one homology sequence or homology arm, such as two homology arms, to direct the integration of a mutation or insertion sequence into a target site within the genome of a plant via homologous recombination, wherein the homology sequence or homology arm(s) are identical or complementary, or have a percent identity or percent complementarity, to a sequence at or near the target site within the genome of the plant. When a donor template comprises homology arm(s) and an insertion sequence, the homology arm(s) will flank or surround the insertion sequence of the donor template. In an aspect, a donor template comprises a premature stop codon in a br2 nucleic acid sequence. In an aspect, a donor template comprises at least one homology arm that targets an endogenous br2 locus.

A donor template may be linear or circular and may be single-stranded or double-stranded. A donor template may be delivered to the cell as a naked nucleic acid (e.g., via particle bombardment), as a complex with one or more delivery agents (e.g., liposomes, proteins, poloxamers, T-strand encapsulated with proteins, etc.), or contained in a bacterial or viral delivery vehicle, such as, for example, Agrobacterium tumefaciens or a geminivirus, respectively. An insertion sequence of a donor template or insertion sequence provided herein may comprise a transcribable DNA sequence or segment that may be transcribed into all or a portion of an RNA molecule, such as an antisense sequence or portion of a RNA molecule.

As used herein, the terms “suppress,” “suppression,” “inhibit,” “inhibition,” “inhibiting”, and “downregulation” with regard to a target gene (e.g., an endogenous gene) expression refer to a lowering, reduction or elimination of the expression level of a mRNA and/or protein encoded by a target gene in a plant, plant cell or plant tissue at one or more stage(s) of plant development, as compared to the expression level of such target mRNA and/or protein in a wild-type or control plant, cell or tissue at the same stage(s) of plant development. According to some embodiments, a modified plant is provided having a br2 gene expression level that is reduced in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified plant is provided having a br2 gene expression level that is reduced in at least one plant tissue by 5%-20%, 5%-25%, 5%-30%, 5%-40%, 5%-50%, 5%-60%, 5%-70%, 5%-75%, 5%-80%, 5%-90%, 5%-10%, 75%-100%, 50%-100%, 50%-90%, 50%-75%, 25%-75%, 30%-80%, or 10%-75%, as compared to a control plant. According to some embodiments, a modified plant is provided having a br2 mRNA level that is reduced in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified or transgenic plant is provided having a br2 mRNA expression level that is reduced in at least one plant tissue by 5%-20%, 5%-25%, 5%/6-30%, 5%-40%, 5/6-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant. According to some embodiments, a modified plant is provided having a Br2 protein expression level that is reduced in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified plant is provided having a Br2 protein expression level that is reduced in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant.

As used herein, an “intervening region” or “intervening sequence” refers to a polynucleotide sequence between a physically linked first polynucleotide sequence and second polynucleotide sequence. The intervening sequence may form a loop, and the first and second sequences may hybridize to form a stem, of a stem-loop or hairpin structure. In one aspect, an intervening region or intervening sequence comprises at least 1, at least 2, at least 3, at least 4, at least 5, at least 10, at least 25, at least 50, at least 100, at least 150, at least 200, at least 250, at least 500, at least 1000, at least 1250, at least 1500, at least 1750, at least 2000, at least 2500, at least 3000, at least 4000, at least 5000, at least 6000, at least 7000, at least 8000, at least 9000, at least 10,000, at least 15,000, at least 20,000, at least 25,000, or at least 50,000 nucleotides. In one aspect, an intervening region or intervening sequence comprises a DNA sequence. In one aspect, an intervening region or intervening sequence comprises an RNA sequence. In one aspect, an intervening region or intervening sequences comprises an endogenous or native nucleic acid sequence. In another aspect, an intervening region or intervening sequences comprises a transgenic or exogenous nucleic acid sequence. In one aspect, an intervening region or intervening sequences comprises an endogenous or native nucleic acid sequence and a transgenic or exogenous nucleic acid sequence.

A wild-type genomic DNA sequence of the br2 locus from a reference genome of corn or maize is provided in SEQ ID NO: 1. A wild-type cDNA sequence for the br2 locus from the reference genome is provided in SEQ ID NO: 50. A wild-type amino acid sequence encoded by the br2 gene and SEQ ID NO: 50 (and coding sequence/exons of SEQ ID NO: 1) is provided in SEQ ID NO: 51. For the br2 genomic locus, nucleotides 1-954 of SEQ ID NO: 1 are upstream of the br2 5′-UTR; nucleotides 955-1000 of SEQ ID NO: 1 correspond to the 5′-UTR of the br2 gene; nucleotides 1001-1604 of SEQ ID NO: 1 correspond to the first exon of the br2 gene; nucleotides 1605-1747 of SEQ ID NO: 1 correspond to the first intron of the br2 gene; nucleotides 1748-2384 of SEQ ID NO: 1 correspond to the second exon of the br2 gene; nucleotides 2385-2473 of SEQ ID NO: 1 correspond to the second intron of the br2 gene; nucleotides 2474-2784 of SEQ ID NO: 1 correspond to the third exon of the br2 gene; nucleotides 2785-3410 of SEQ ID NO: 1 correspond to the third intron of the br2 gene; nucleotides 3411-3640 of SEQ ID NO: 1 correspond to the fourth exon of the br2 gene; nucleotides 3641-5309 of SEQ ID NO: 1 correspond to the fourth intron of the br2 gene; nucleotides 5310-7667 of SEQ ID NO: 1 correspond to the fifth exon of the br2 gene; and nucleotides 7668-8029 of SEQ ID NO: 1 correspond to the 3′-UTR of the br2 gene. SEQ ID NO: 1 also provides 638 nucleotides downstream of the 3′-UTR of the br2 gene (nucleotides 8030-8667 of SEQ ID NO: 1).

In an aspect, an endogenous br2 locus comprises a nucleotide sequence that is at least 90% identical to SEQ ID NO: 1. In an aspect, an endogenous br2 locus comprises a nucleotide sequence that is at least 91% identical to SEQ ID NO: 1. In an aspect, an endogenous br2 locus comprises a nucleotide sequence that is at least 92% identical to SEQ ID NO: 1. In an aspect, an endogenous br2 locus comprises a nucleotide sequence that is at least 93% identical to SEQ ID NO: 1. In an aspect, an endogenous br2 locus comprises a nucleotide sequence that is at least 94% identical to SEQ ID NO: 1. In an aspect, an endogenous br2 locus comprises a nucleotide sequence that is at least 95% identical to SEQ ID NO: 1. In an aspect, an endogenous br2 locus comprises a nucleotide sequence that is at least 96% identical to SEQ ID NO: 1. In an aspect, an endogenous br2 locus comprises a nucleotide sequence that is at least 97% identical to SEQ ID NO: 1. In an aspect, an endogenous br2 locus comprises a nucleotide sequence that is at least 98% identical to SEQ ID NO: 1. In an aspect, an endogenous br2 locus comprises a nucleotide sequence that is at least 99% identical to SEQ ID NO: 1. In an aspect, an endogenous br2 locus comprises a nucleotide sequence that is 100% identical to SEQ ID NO: 1.

Brachytic2 (br2) is a homologue of the Arabidopsis thaliana gene ATP BINDING CASSETTE TYPE B1 (ABCB1) auxin transporter. See Knöller et al., J. Exp. Botany, 61:3689-3696 (2010). Br2 has been demonstrated to function in the export of auxin from intercalary meristems. See Knöller et al. Intercalary meristems form at the base of nodes and leaf blades in grasses such as corn. Without being limited by any theory, it has been hypothesized that auxin exported from intercalary meristems promotes the elongation of cells between nodes, allowing for rapid vertical growth of some grass species (e.g., corn). It has been shown that some recessive mutant alleles of br2 can be effective in achieving a short stature plant height in corn due to a shortening of the internode length without a corresponding reduction in the number of internodes or the number and size of other organs. See PCT Application No. PCT/US2016/029492, published as WO/2016/176286 and U.S. Pat. No. 10,472,684, respectively. However, these short stature phenotypes were observed with plants that were homozygous for a recessive br2 mutation. Thus, both parents must carry the recessive allele or mutation for the progeny or hybrid corn plant to have the short stature phenotype. In contrast, the present disclosure describes br2 mutant alleles that can produce a short stature phenotype in corn or maize plants when present in a heterozygous state. These dominant or semi-dominant alleles of the br2 gene can be present in only one of the parent plants to produce the short stature phenotype in their progeny or hybrid plants, although such alleles may also be carried by both parents.

As understood in the art, a dominant allele is an allele that masks the contribution of a second allele (e.g., a wild-type allele) at the same locus. If the masking of the other allele is partial or incomplete, the dominant allele may be described as being semi-dominant. As used herein, a dominant allele(s) or trait(s) include(s) any semi-dominant allele(s) or trait(s) of a gene or locus. It is possible in some cases for a dominant allele at one locus to also have a dominant effect over a gene(s) or allele(s) at another locus/loci. Dominant negative alleles, or anti-morphs, are alleles that produce altered or modified gene products that act to oppose or reduce wild-type allelic function. For example, a dominant negative allele can reduce, abrogate or suppress the normal function of a wild-type allele or gene product in a heterozygous state.

A variety of mechanisms are possible for a dominant or semi-dominant allele (e.g., dominant negative allele) to exert its masking effect on another copy or allele for the same gene or locus. Without being bound by theory, one such mechanism is that a modified br2 gene containing an inversion or antisense sequence relative to a sense sequence of the br2 gene may suppress the expression of another copy or allele of the br2 gene in a dominant negative manner via suppression or RNAi mechanisms. In some embodiments, if the inversion or antisense sequence is inserted adjacent or near to a complementary sequence in the br2 gene, the inversion or antisense sequence may form a hairpin or stem-loop structure with the nearby complementary sequence within the br2 gene, which may operate in a dominant negative manner on another copy or allele of the br2 gene via suppression or RNAi mechanisms. Without being bound by theory, a mutant or edited allele of a br2 gene or locus may affect the expression level(s) of another copy or allele of the br2 gene or locus through other mechanisms, such as nonsense mediated decay, non-stop decay, no-go decay, DNA or histone methylation or other epigenetic changes, inhibition or decreased efficiency of transcription and/or translation, ribosomal interference, interference with mRNA processing or splicing, and/or ubiquitin-mediated protein degradation via the proteasome. See, e.g., Nickless, A. et al., “Control of gene expression through the nonsense-mediated RNA decay pathway”, Cell Biosci 7:26 (2017); Karamyshev, A. et al., “Lost in Translation: Ribosome-Associated mRNA and Protein Quality Controls”, Frontiers in Genetics 9:431(2018); Inada, T., “Quality controls induced by aberrant translation”, Nucleic Acids Res 48:3 (2020); and Szadeczky-Kardoss, I. et al., “The nonstop decay and the RNA silencing systems operate cooperatively in plants”, Nucleic Acids Res 46:9 (2018), the entire contents and disclosures of which are incorporated herein by reference. Each of these different mechanisms may act alternatively or in addition to RNA interference (RNAi), transcriptional gene silencing, and/or post transcriptional gene silencing (PTGS) mechanisms. See, e.g., Wilson, R. C. et al., “Molecular Mechanisms of RNA Interference”, Annu Rev Biophysics 42:217-39 (2013); and Guo, Q. et al., “RNA Silencing in Plants: Mechanism, Technologies and Applications in Horticulture Crops”, Current Genomics 17:476-489 (2016), the entire contents and disclosures of which is incorporated herein by reference. Some of the above mechanisms may reduce expression of the edited allele itself, while others may also reduce the expression of other copy/-ies or allele(s) of the endogenous br2 locus or gene. Indeed, it is envisioned that the presence of an antisense or inversion sequence, such as a DNA segment encoding an antisense RNA sequence, in an edited endogenous br2 gene, locus or allele may not only reduce or eliminate its own expression and/or activity level, but may also have a dominant or semi-dominant effect(s) on the other copy/-ies or allele(s) of the endogenous br2 locus or gene. Such dominant or semi-dominant effect(s) on the br2 gene may operate through non-canonical suppression mechanisms that do not involve RNAi and/or formation of targeted small RNAs at a significant or detectable level, even if the formation of small RNA molecules is not shown.

Without being limited by any theory, a mutant or edited allele of a br2 gene or locus may comprise a deletion of all or part of the br2 gene or locus and/or a premature stop codon in the coding sequence of the br2 gene or locus. Such deletion or premature stop codon may cause an altered or truncated Br2 protein or polypeptide fragment to be expressed, encoded and translated from the mutant or edited allele of a br2 gene or locus, which may not only have a loss-of-function but also interfere with the function and/or expression of a Br2 protein expressed from another copy or allele of the br2 gene or locus (e.g., a wild-type copy or allele of the br2 gene or locus) in a dominant or semi-dominant manner. Without being limited by theory, an altered or truncated Br2 protein expressed from a mutant or edited allele of a br2 gene or locus comprising a deletion of all or part of the br2 gene or locus and/or a premature stop codon in the coding sequence of the br2 gene or locus may interfere with the function and/or expression of a Br2 protein expressed from another copy or allele of the br2 gene or locus if the Br2 proteins bind to, or form complexes with, each other and/or other proteins, which can affect the function of the Br2 protein expressed from the other copy or allele of the br2 gene or locus, such as by affecting or reducing the function of the bound proteins or complex and/or competing against the binding of the Br2 protein expressed from the other copy or allele of the br2 gene or locus.

According to some embodiments, an endogenous gene can be edited or engineered to express a truncated protein relative to a wild type protein by the introduction of a premature stop codon into the coding sequence and the encoded mRNA transcript of the endogenous gene. Without being bound by theory, a truncated Br2 protein expressed from an edited endogenous br2 gene comprising a premature stop codon may not only be non-functional or have reduced function, but also interfere with the functioning of a wild type Br2 protein to act in a dominant or semi-dominant manner. In an aspect, a premature stop codon within an mRNA transcript results in translation of a truncated protein as compared to a control mRNA transcript that lacks the premature stop codon. As used herein, a “stop codon” refers to a nucleotide triplet within an mRNA transcript that signals a termination of protein translation. A “premature stop codon” refers to a stop codon positioned earlier (e.g., on the 5′-side) than the normal stop codon position in an endogenous mRNA transcript. A stop codon is a nucleotide triplet in a mRNA that signals the termination of protein translation from the mRNA. Without being limiting, several stop codons are known in the art, including “UAG,” “UAA,” “UGA,” “TAG,” “TAA,” and “TGA.” In an aspect, a premature stop codon can arise from a frameshift mutation. Frameshift mutations can be caused by the insertion or deletion of one or more nucleotides in a protein-coding sequence. In an aspect, a premature stop codon can arise from a substitution, missense or nonsense mutation. In an aspect, a nonsense, missense or frameshift mutation provided herein is located in an exon of a br2 gene. In an aspect, a substitution, insertion or deletion provided herein is located in a gene element selected from the group consisting of an exon and an intron/exon splice site. A substitution, insertion or deletion provided herein can generate a protein with one or more, two or more, three or more, four or more, five or more, six or more, seven or more, eight or more, nine or more, or ten or more nonsense mutations. According to present embodiments, a premature stop codon may be introduced into the coding sequence of an endogenous br2 gene via a targeted editing technique and/or site-directed integration. The premature stop codon may be generated via imperfect DNA repair following a double strand break introduced into a br2 gene, or via template-assisted repair following introduction of the double strand break using a DNA donor template comprising the premature stop codon. Such a DNA donor template may further comprise one or more flanking homologous arms or sequences that are identical, homologous or complementary to a corresponding sequence of the endogenous br2 gene to help promote recombination between the donor template and the target site in the endogenous br2 gene for insertion of a sequence comprising the premature stop codon at the desired target site.

In an aspect, a premature stop codon is positioned within the first exon of an endogenous br2 locus. In an aspect, a premature stop codon is positioned within the second exon of an endogenous br2 locus. In an aspect, a premature stop codon is positioned within the third exon of an endogenous br2 locus. In an aspect, a premature stop codon is positioned within the fourth exon of an endogenous br2 locus. In an aspect, a premature stop codon is positioned within the fifth exon of an endogenous br2 locus.

In an aspect, a mutant allele provided herein encodes a truncated protein as compared to SEQ ID NO: 51. As used herein, a “truncated” protein or polypeptide comprises at least one fewer amino acid(s) as compared to an endogenous control protein or polypeptide. For example, if endogenous Protein A comprises 100 amino acids, a truncated version of Protein A can comprise between 1 and 99 amino acids.

In an aspect, this disclosure provides a modified corn plant, or plant part thereof, comprising a premature stop codon within a nucleic acid sequence encoding a Brachytic2 protein as compared to a nucleic acid sequence of a control corn plant or plant part thereof. In an aspect, this disclosure provides a modified corn plant, or plant part thereof, comprising a premature stop codon within a nucleic acid sequence encoding a Brachytic2 protein. In an aspect, this disclosure provides a modified corn plant, or plant part thereof, comprising a truncated Brachytic2 protein encoded by a nucleic acid sequence comprising a premature stop codon as compared to a wildtype or control nucleic acid sequence. In an aspect, this disclosure provides a modified corn plant, or plant part thereof, comprising a premature stop codon in a nucleic acid sequence as compared to SEQ ID NO: 50.

In an aspect, a premature stop codon is positioned within a region of a br2 mRNA transcript selected from the group consisting of the first exon, the second exon, the third exon, the fourth exon, and the fifth exon.

In an aspect, a truncated Br2 protein sequence comprises fewer than 1378 amino acids. In an aspect, a truncated Br2 protein sequence comprises fewer than 1375 amino acids. In an aspect, a truncated Br2 protein sequence comprises fewer than 1350 amino acids. In an aspect, a truncated Br2 protein sequence comprises fewer than 1300 amino acids. In an aspect, a truncated Br2 protein sequence comprises fewer than 1200 amino acids. In an aspect, a truncated Br2 protein sequence comprises fewer than 1100 amino acids. In an aspect, a truncated Br2 protein sequence comprises fewer than 1000 amino acids. In an aspect, a truncated Br2 protein sequence comprises fewer than 900 amino acids. In an aspect, a truncated Br2 protein sequence comprises fewer than 800 amino acids. In an aspect, a truncated Br2 protein sequence comprises fewer than 700 amino acids. In an aspect, a truncated Br2 protein sequence comprises fewer than 600 amino acids. In an aspect, a truncated Br2 protein sequence comprises fewer than 500 amino acids. In an aspect, a truncated Br2 protein sequence comprises fewer than 400 amino acids. In an aspect, a truncated Br2 protein sequence comprises fewer than 300 amino acids. In an aspect, a truncated Br2 protein sequence comprises fewer than 200 amino acids. In an aspect, a truncated Br2 protein sequence comprises fewer than 100 amino acids. In an aspect, a truncated Br2 protein sequence comprises fewer than 50 amino acids.

In an aspect, a truncated Br2 protein sequence comprises between 1 amino acid and 1378 amino acids. In an aspect, a truncated Br2 protein sequence comprises between 25 amino acids and 1378 amino acids. In an aspect, a truncated Br2 protein sequence comprises between 50 amino acids and 1378 amino acids. In an aspect, a truncated Br2 protein sequence comprises between 100 amino acids and 1378 amino acids. In an aspect, a truncated Br2 protein sequence comprises between 250 amino acids and 1378 amino acids. In an aspect, a truncated Br2 protein sequence comprises between 500 amino acids and 1378 amino acids. In an aspect, a truncated Br2 protein sequence comprises between 750 amino acids and 1378 amino acids. In an aspect, a truncated Br2 protein sequence comprises between 1000 amino acids and 1378 amino acids. In an aspect, a truncated Br2 protein sequence comprises between 1250 amino acids and 1378 amino acids. In an aspect, a truncated Br2 protein sequence comprises between 100 amino acids and 1000 amino acids. In an aspect, a truncated Br2 protein sequence comprises between 250 amino acids and 1000 amino acids. In an aspect, a truncated Br2 protein sequence comprises between 500 amino acids and 1000 amino acids. In an aspect, a truncated Br2 protein sequence comprises between 750 amino acids and 1000 amino acids. In an aspect, a truncated Br2 protein sequence comprises between 1000 amino acids and 1375 amino acids.

In an aspect, a premature stop codon is introduced to an endogenous br2 locus via a targeted editing technique. In an aspect, this disclosure provides a method for producing a mutant allele of an endogenous Brachytic2 (br2) locus, the method comprising: (a) generating at least a double-stranded break (DSB) in the endogenous br2 locus in at least one corn cell using a targeted editing technique; and (b) identifying at least one corn cell, corn seed or corn plant from the at least one corn cell in step (a) comprising a premature stop codon in the coding sequence of the endogenous br2 locus. In an aspect, the method further comprises regenerating at least one corn plant from the at least one corn cell identified in step (b).

Creation of dominant alleles that work in a heterozygous state, can speed up effective trait development, deployment, and launch of gene editing-derive products in hybrid crops such as corn. Dominant alleles have the potential advantage of providing a positive or beneficial plant trait in a heterozygous state—e.g., when present in a single copy. As a result, the dominant mutant allele can be introduced through crossing into a progeny plant from a single parent without having to introduce the allele from both parent plants as with a recessive trait or allele. The present disclosure provides methods and compositions to selectively mutate or edit a genome of a corn plant to create a dominant allele that produces a beneficial trait in a plant.

In an aspect, this disclosure provides a modified corn plant, or a method for producing a modified corn plant, where the modified corn plant has a dominant allele at the endogenous br2 locus or gene that causes the modified corn plant to have a beneficial phenotype or trait relative to a wild-type or control plant. Such dominant allele of the endogenous br2 locus or gene can modify, alter, reduce and/or mask a trait associated with a wild-type allele through one or more mechanisms as described herein.

In an aspect, this disclosure provides a modified corn plant, or a method for producing a modified corn plant, where the modified corn plant comprises a mutant or edited allele of an endogenous br2 gene or locus comprising a deletion of all or part of the br2 gene or locus and/or a premature stop codon in the coding sequence of the br2 gene or locus. In an aspect, this disclosure provides a modified corn plant, or plant part thereof, comprising a mutant allele at the endogenous br2 locus, wherein the mutant allele comprises a DNA insertion encoding an antisense RNA sequence, wherein the mutant allele produces a RNA transcript comprising the antisense RNA sequence. In an aspect, an antisense RNA sequence is capable of suppressing the expression of a wild-type allele of the br2 locus.

In an aspect, an antisense RNA sequence encoded by the DNA insertion is at least 70%, at least 75%, at least 80%, at least 85%, at least 90%, at least 95%, at least 96%, at least 97%, at least 98%, at least 99%, or 100% complementary to at least 20, at least 25, at least 30, at least 40, at least 50, at least 75, at least 100, at least 150, at least 200, at least 300, at least 400, at least 500, at least 750, or at least 1000 consecutive nucleotides of one or more of SEQ ID NOs: 1 and 50. In another aspect, an antisense RNA sequence encoded by the DNA insertion is complementary to a coding sequence and/or one or more exon, intron and/or untranslated region (UTR) sequences of the br2 gene or locus. In another aspect, an antisense RNA sequence encoded by the DNA insertion is at least 70%, at least 75%, at least 80%, at least 85%, at least 90%, at least 95%, at least 96%, at least 97%, at least 98%, at least 99%, or 100% complementary to at least 20, at least 25, at least 30, at least 40, at least 50, at least 75, at least 100, at least 150, at least 200, at least 300, at least 400, at least 500, at least 750, or at least 1000 consecutive nucleotides of a sequence comprising one or more of nucleotides 955-1000 of SEQ ID NO: 1; nucleotides 1001-1604 of SEQ ID NO: 1; nucleotides 1605-1747 of SEQ ID NO: 1; nucleotides 1748-2384 of SEQ ID NO: 1; nucleotides 2385-2473 of SEQ ID NO: 1; nucleotides 2474-2784 of SEQ ID NO: 1; nucleotides 2785-3410 of SEQ ID NO: 1; nucleotides 3411-3640 of SEQ ID NO: 1; nucleotides 3641-5309 of SEQ ID NO: 1; nucleotides 5310-7667 of SEQ ID NO: 1; and/or nucleotides 7668-8029 of SEQ ID NO: 1. In an aspect, an RNA transcript from a mutant allele at the endogenous br2 locus is able to suppress the expression of a wild-type allele of the br2 locus. In another aspect, an RNA transcript from a first mutant allele at the endogenous br2 locus is able to suppress the expression of a second mutant allele of the br2 locus. In an aspect, a mutant allele of an endogenous br2 locus suppresses the expression of a wild-type allele of the endogenous br2 locus. In an aspect, a mutant allele product of an endogenous br2 locus disrupts the function of a wild-type allele product of the endogenous br2 locus. In an aspect, a “product” of a mutant allele is a mRNA transcript. In an aspect, a “product” comprises an antisense RNA. In an aspect, a “product” of a mutant allele is a protein.

In another aspect, this disclosure provides a modified corn plant, or plant part thereof, comprising a single mutant allele at the endogenous br2 locus, wherein the single mutant allele comprises a DNA segment producing an antisense RNA sequence. In another aspect, this disclosure provides a modified corn plant, or plant part thereof, comprising two mutant alleles at the endogenous br2 locus comprising a first mutant allele and a second mutant allele, wherein each mutant allele comprises a DNA segment producing or encoding an antisense RNA sequence. In an aspect, a DNA insertion or segment is at least 70%, at least 75%, at least 80%, at least 85%, at least 90%, at least 95%, at least 96%, at least 97%, at least 98%, at least 99%, or 100% complementary to at least 20, at least 25, at least 30, at least 40, at least 50, at least 75, at least 100, at least 150, at least 200, at least 300, at least 400, at least 500, at least 750, or at least 1000 consecutive nucleotides of one or more of SEQ ID NOs: 1 and 50.

Further provided herein are methods of generating dominant or semi-dominant alleles of a br2 gene using targeted editing techniques. Also provided herein are cells generated by such methods and compositions used in such methods. The instant description further provides modified plants regenerated from cells subjected to the methods provided herein. In one aspect, a dominant or semi-dominant allele provided herein is able to suppress the expression of a wild-type (or mutant) allele of a br2 locus or gene in a heterozygous state.

In an aspect, the present disclosure provides a modified corn plant, or plant part thereof, comprising a mutant allele of the endogenous br2 locus, where the mutant allele comprises a DNA segment inserted into the endogenous br2 locus, where the DNA segment encodes an antisense RNA sequence that is at least 70%, at least 75%, at least 80%, at least 85%, at least 90%, at least 95%, at least 96%, at least 97%, at least 98%, at least 99%, or 100% complementary to at least 20, at least 25, at least 30, at least 40, at least 50, at least 75, at least 100, at least 150, at least 200, at least 300, at least 400, at least 500, at least 750, or at least 1000 consecutive nucleotides of one or more of SEQ ID NOs: 1 and 50, and where the mutant allele produces a RNA transcript comprising the antisense RNA sequence. In an aspect, a br2 mutant allele is able to suppress the expression of a wild-type (or mutant) allele of the endogenous br2 locus. In another aspect, a br2 mutant allele suppresses the expression of a wild-type (or mutant) allele of the endogenous br2 locus. In a further aspect, a RNA transcript produced by a br2 mutant allele provided here, further comprises one or more sequence elements of the endogenous br2 locus selected from the group consisting of 5′ UTR, first exon, first intron, second exon, second intron, third exon, third intron, fourth exon, fourth intron, fifth exon, 3′ UTR, and any portion thereof.

In an aspect, an inserted DNA segment in a br2 mutant allele comprises a nucleotide sequence originating from the endogenous br2 locus. In another aspect, an inserted DNA segment in a br2 mutant allele corresponds to an inverted genomic fragment of the endogenous br2 locus. In another aspect, an inserted DNA segment in a br2 mutant allele corresponds to a DNA sequence fragment from a donor template.

In an aspect, at least a portion of the antisense RNA sequence in a RNA transcript produced by a br2 mutant allele, is at least 70%, at least 75%, at least 80%, at least 85%, at least 90%, at least 95%, at least 96%, at least 97%, at least 98%, at least 99%, or 100% complementary to a corresponding endogenous sequence of the RNA transcript. In another aspect, a corresponding endogenous sequence of the RNA transcript is at least 85%, at least 90%, at least 95%, at least 96%, at least 97%, at least 98%, at least 99%, or 100% identical to at least 20, at least 25, at least 30, at least 40, at least 50, at least 75, at least 100, at least 150, at least 200, at least 300, at least 400, at least 500, at least 750, or at least 1000 consecutive nucleotides of one or more of SEQ ID NOs: 1 and 50. In a further aspect, an antisense RNA sequence encoded by an inserted DNA segment in a br2 mutant allele hybridizes to the corresponding endogenous sequence of a RNA transcript produced by the br2 mutant allele.

In an aspect, a br2 mutant allele provided here comprises an inserted DNA segment having a length of at least 15, 16, 17, 18, 19, 20, 21, 22, 23, 24, 25, 30, 35, 40, 45, 50, 75, 100, 125, 150, 175, 200, 250, 300, 400, 500, 750, 1000, 1500, 2000, 2500, or 3000 nucleotides. In another aspect, a br2 mutant allele comprises an inserted DNA segment having a length of at most 25, 50, 100, 150, 200, 250, 300, 400, 500, 750, 1000, 1500, 2000, 2500, or 3000 nucleotides. In another aspect, a br2 mutant allele comprises an inserted DNA segment having a length of between 20 and 50, between 50 and 100, between 100 and 200, between 200 and 300, between 300 and 400, between 400 and 500, between 500 and 750, between 750 and 1000, between 1000 and 1500, between 1500 and 2000, between 2000 and 3000, or between 3000 and 4000 nucleotides. In another aspect, a br2 mutant allele comprises an inserted DNA segment having a length between 20 and 4000, between 50 and 4000, between 100 and 4000, between 200 and 4000, between 300 and 4000, between 400 and 4000, between 500 and 4000, between 750 and 4000, between 1000 and 4000, 5 between 1500 and 4000, or between 2000 and 4000 nucleotides. In another aspect, a br2 mutant allele comprises an inserted DNA segment having a length between 20 and 100, between 20 and 200, between 20 and 300, between 20 and 400, between 20 and 500, between 20 and 750, between 20 and 1000, between 20 and 1500, between 20 and 2000, between 20 and 3000, or between 20 and 4000 nucleotides. In another aspect, a br2 mutant allele comprises an inserted DNA segment having a length between 20 and 3000, between 50 and 2000, between 100 and 1500, between 200 and 1000, between 300 and 750, or between 400 and 750 nucleotides.

In an aspect, a br2 mutant allele comprises a DNA segment inserted near or adjacent to a corresponding endogenous DNA segment of the endogenous br2 locus. In another aspect, an antisense RNA sequence encoded by an inserted DNA segment hybridizes to a corresponding endogenous sequence of the RNA transcript encoded by the corresponding endogenous DNA segment. In a further aspect, an antisense RNA sequence forms a stem-loop structure with the corresponding endogenous sequence of the RNA transcript.

In an aspect, a br2 mutant allele comprises an inserted DNA segment and a corresponding endogenous DNA segment separated by an intervening DNA sequence. In another aspect, an intervening DNA sequence has a length of at least 1, 2, 3, 4, 5, 6, 7, 8, 9, 10, 11, 12, 13, 14, 15, 20, 25, 30, 40, 50, 75, 100, 150, 200, 250, 300, 400, 500, 750, 1000, 1500, 2000, 3000, or 4000 consecutive nucleotides. In a further aspect, an intervening sequence has at most 1, 2, 3, 4, 5, 6, 7, 8, 9, 10, 11, 12, 13, 14, 15, 20, 25, 30, 40, 50, 75, 100, 150, 200, 250, 300, 400, 500, 750, 1000, 1500, 2000, 3000, or 4000 consecutive nucleotides. In another aspect, an intervening DNA sequence has a length between 2 and 10, between 10 and 20, between 20 and 50, between 50 and 100, between 100 and 200, between 200 and 300, between 300 and 400, between 400 and 500, between 500 and 750, between 750 and 1000, between 1000 and 1500, between 1500 and 2000, between 2000 and 3000, or between 3000 and 4000 nucleotides. In another aspect, an intervening DNA sequence has a length between 2 and 4000, between 10 and 4000, between 20 and 4000, between 50 and 4000, between 100 and 4000, between 200 and 4000, between 300 and 4000, between 400 and 4000, between 500 and 4000, between 750 and 4000, between 1000 and 4000, between 1500 and 4000, or between 2000 and 4000 nucleotides. In another aspect, an intervening DNA sequence has a length between 10 and 20, between 10 and 50, between 10 and 100, between 10 and 200, between 10 and 300, between 10 and 400, between 10 and 500, between 10 and 750, between 10 and 1000, between 10 and 1500, between 10 and 2000, between 10 and 3000, or between 10 and 4000 nucleotides. In another aspect, an intervening DNA sequence has a length between 20 and 3000, between 50 and 2000, between 100 and 1500, between 200 and 1000, between 300 and 750, or between 400 and 750 nucleotides.

In a further aspect, an intervening DNA sequence encodes an intervening RNA sequence between the antisense RNA sequence and the corresponding endogenous sequence of the RNA transcript. In an aspect, an intervening RNA sequence forms the loop portion of a stem-loop structure of a RNA transcript produced by a br2 mutant allele. In another aspect, a stem-loop secondary structure contains a near-perfect-complement stem with mismatches. In a further aspect, a stem-loop secondary structure contains a perfect-complement stem with no mismatches. In another aspect, an intervening DNA sequence comprises a native sequence of the endogenous br2 locus. In an aspect, an intervening DNA sequence comprises an exogenous sequence inserted into the endogenous br2 locus. In another aspect, an intervening DNA sequence comprises an intron sequence. In a further aspect, an intervening DNA sequence does not contain an intron sequence.

In an aspect, a br2 mutant allele comprises an inserted DNA segment located upstream of the corresponding endogenous DNA segment. In another aspect, a br2 mutant allele comprises an inserted DNA segment is located downstream of the corresponding endogenous DNA segment.

In an aspect, a br2 mutant allele comprises an inserted DNA segment within a region selected from the group consisting of 5′ untranslated region (UTR), first exon, first intron, second exon, second intron, third exon, third intron, fourth exon, fourth intron, fifth exon, and 3′ UTR of the endogenous br2 locus, and a combination thereof. In an aspect, a br2 mutant allele comprises an inserted DNA segment at a genomic site recognized by a targeted editing technique to create a double-stranded break (DSB).

In an aspect, a br2 mutant allele further comprises a deletion of at least one portion of the endogenous br2 locus.

In an aspect, this disclosure provides a modified corn plant, or plant part thereof, comprising a deletion within an endogenous br2 locus as compared to a control corn plant or plant part thereof. In an aspect, this disclosure provides a modified corn plant, or plant part thereof, comprising a deletion of or within at least one exon of an endogenous br2 locus as compared to a control corn plant or plant part thereof. In an aspect, this disclosure provides a modified corn plant, or plant part thereof, comprising a deletion of at least one nucleotide from at least one exon of an endogenous br2 locus as compared to a control corn plant or plant part thereof.

In an aspect, a modified corn plant, or plant part thereof, is homozygous for a deletion within an endogenous br2 locus. In an aspect, a modified corn plant, or plant part thereof, is biallelic for a first mutant allele and a second mutant allele each within an endogenous br2 locus. In an aspect, a first mutant allele comprises a deletion and/or an inversion or antisense sequence. In an aspect, a second mutant allele comprises a deletion and/or an inversion or antisense sequence. In an aspect, a modified corn plant, or plant part thereof, is heterozygous for a deletion and/or an inversion or antisense sequence within an endogenous br2 locus.

In an aspect, a deletion within an endogenous br2 locus comprises between 1 nucleotide and 8667 nucleotides, between 1 nucleotide and 8000 nucleotides, between 1 nucleotide and 7000 nucleotides, between 1 nucleotide and 6000 nucleotides, between 1 nucleotide and 5000 nucleotides, between 1 nucleotide and 4000 nucleotides, between 1 nucleotide and 3000 nucleotides, between 1 nucleotide and 2000 nucleotides, between 1 nucleotide and 1000 nucleotides, between 1 nucleotide and 750 nucleotides, between 1 nucleotide and 500 nucleotides, between 1 nucleotide and 250 nucleotides, between 1 nucleotide and 100 nucleotides, between 1 nucleotide and 50 nucleotides, between 10 nucleotide and 8000 nucleotides, between 10 nucleotide and 5000 nucleotides, between 10 nucleotide and 2500 nucleotides, between 10 nucleotide and 1000 nucleotides, between 10 nucleotide and 100 nucleotides, between 100 nucleotide and 8000 nucleotides, between 100 nucleotide and 5000 nucleotides, between 100 nucleotide and 2500 nucleotides, between 100 nucleotide and 1000 nucleotides, or between 100 nucleotide and 500 nucleotides. In an aspect, a deletion within an endogenous br2 locus comprises at least 1 nucleotide. In an aspect, a deletion within an endogenous br2 locus comprises at least 2 nucleotides. In an aspect, a deletion within an endogenous br2 locus comprises at least 5 nucleotides. In an aspect, a deletion within an endogenous br2 locus comprises at least 10 nucleotides. In an aspect, a deletion within an endogenous br2 locus comprises at least 20 nucleotides. In an aspect, a deletion within an endogenous br2 locus comprises at least 30 nucleotides. In an aspect, a deletion within an endogenous br2 locus comprises at least 40 nucleotides. In an aspect, a deletion within an endogenous br2 locus comprises at least 50 nucleotides. In an aspect, a deletion within an endogenous br2 locus comprises at least 100 nucleotides. In an aspect, a deletion within an endogenous br2 locus comprises at least 200 nucleotides. In an aspect, a deletion within an endogenous br2 locus comprises at least 300 nucleotides. In an aspect, a deletion within an endogenous br2 locus comprises at least 400 nucleotides. In an aspect, a deletion within an endogenous br2 locus comprises at least 500 nucleotides.

In an aspect, a deletion comprises deletion of at least one nucleotide of the first exon of an endogenous br2 locus. In an aspect, a deletion comprises deletion of at least one nucleotide of the second exon of an endogenous br2 locus. In an aspect, a deletion comprises deletion of at least one nucleotide of the third exon of an endogenous br2 locus. In an aspect, a deletion comprises deletion of at least one nucleotide of the fourth exon of an endogenous br2 locus. In an aspect, a deletion comprises deletion of at least one nucleotide of the fifth exon of an endogenous br2 locus.

In an aspect, a deletion comprises deletion of the first exon of an endogenous br2 locus. In an aspect, a deletion comprises deletion of the second exon of an endogenous br2 locus. In an aspect, a deletion comprises deletion of the third exon of an endogenous br2 locus. In an aspect, a deletion comprises deletion of the fourth exon of an endogenous br2 locus. In an aspect, a deletion comprises deletion of the fifth exon of an endogenous br2 locus.

In an aspect, a deletion comprises deletion of at least one nucleotide of at least one intron of an endogenous br2 locus. In an aspect, a deletion comprises deletion of at least one intron of an endogenous br2 locus. In an aspect, a deletion comprises deletion of at least one nucleotide of the 5′-untranslated region of an endogenous br2 locus. In an aspect, a deletion comprises deletion of the 5′-untranslated region of an endogenous br2 locus. In an aspect, a deletion comprises deletion of at least one nucleotide of the 3′-untranslated region of an endogenous br2 locus. In an aspect, a deletion comprises deletion of the 3′-untranslated region of an endogenous br2 locus.

In an aspect, a deletion comprises a deletion of at least one nucleotide of at least one intron, a deletion of at least one nucleotide of at least two exons, a 5′-untranslated region (UTR), a 3′-UTR, or any combination thereof of an endogenous br2 locus.

In an aspect, a deletion comprises deletion of at least one nucleotide from a first exon and at least one nucleotide from a second exon of an endogenous br2 locus. In an aspect, a deletion comprises deletion of at least one nucleotide from a first exon, at least one nucleotide from a second exon, and at least one nucleotide from a third exon of an endogenous br2 locus. In an aspect, a deletion comprises deletion of at least one nucleotide from a first exon, at least one nucleotide from a second exon, at least one nucleotide from a third exon, and at least one nucleotide from a fourth exon of an endogenous br2 locus. In an aspect, a deletion comprises deletion of at least one nucleotide from a first exon, at least one nucleotide from a second exon, at least one nucleotide from a third exon, at least one nucleotide from a fourth exon, and at least one nucleotide from a fifth exon of an endogenous br2 locus.

In an aspect, a deletion comprises a deletion of a first exon and a second exon from an endogenous br2 locus. In an aspect, a first deleted exon and a second deleted exon are contiguous. In an aspect, a first deleted exon and a second deleted exon are not contiguous. In an aspect, a deletion comprises deletion of a first exon and a second exon from an endogenous br2 locus. In an aspect, a deletion comprises deletion of a first exon, a second exon, and a third exon from an endogenous br2 locus. In an aspect, a deletion comprises deletion of a first exon, a second exon, a third exon, and a fourth exon from an endogenous br2 locus. In an aspect, a deletion comprises deletion of a first exon, a second exon, a third exon, a fourth exon, and a fifth exon from an endogenous br2 locus.

In an aspect, this disclosure provides a method for producing a mutant allele of an endogenous Brachytic2 (br2) locus, the method comprising (a) generating at least a first double-stranded break (DSB) and a second DSB in the endogenous br2 locus in at least one corn cell using a targeted editing technique; and (b) identifying at least one corn cell from step (a) comprising a deletion of the endogenous br2 locus between the first DSB and the second DSB.

In an aspect, a br2 mutant allele comprises an inserted DNA segment, where the sense strand of the inserted DNA segment comprises a sequence at least 70%, at least 75%, at least 80%, at least 85%, at least 90%, at least 95%, at least 96%, at least 97%, at least 98%, at least 99%, or 100% complementary to an exon sequence, or a portion thereof, of the endogenous br2 locus. In another aspect, a br2 mutant allele comprises an inserted DNA segment, where the sense strand of the inserted DNA segment comprises a sequence at least 70%, at least 75%, at least 80%, at least 85%, at least 90%, at least 95%, at least 96%, at least 97%, at least 98%, at least 99%, or 100% complementary to an untranslated region (UTR) sequence, or a portion thereof, of the endogenous br2 locus. In a further aspect, a br2 mutant allele comprises an inserted DNA segment, where the sense strand of the inserted DNA segment comprises a sequence at least 70%, at least 75%, at least 80%, at least 85%, at least 90%, at least 95%, at least 96%, at least 97%, at least 98%, at least 99%, or 100% complementary to at least a portion of an exon sequence and at least a portion of an intron sequence of the endogenous br2 locus, the exon sequence and the intron sequence being contiguous within the endogenous locus.

In an aspect, a br2 mutant allele comprises an inserted DNA segment comprising a sequence having at least at least 70%, at least 75%, at least 80%, at least 85%, at least 90%, at least 95%, at least 96%, at least 97%, at least 98%, at least 99%, or 100% identity or complementarity to one or more of SEQ ID Nos: 1 and 50.

In an aspect, a modified corn plant, or plant part thereof, is homozygous for a mutant allele at the endogenous br2 locus. In another aspect, a modified corn plant, or plant part thereof, is heterozygous for a mutant allele at the endogenous br2 locus. In another aspect, a modified corn plant, or plant part thereof, is trans heterozygous or biallelic at the endogenous br2 locus.

In an aspect, the present disclosure provides a method for producing a mutant allele of the endogenous br2 locus, the method comprising: (a) generating a first double-stranded break (DSB) in the endogenous br2 locus in a corn cell using a targeted editing technique; and (b) inserting at the first DSB a DNA segment using a targeted editing technique, where the DNA segment encodes an antisense RNA sequence that is at least 70%, at least 75%, at least 80%, at least 85%, at least 90%, at least 95%, at least 96%, at least 97%, at least 98%, at least 99%, or 100% complementary to at least 20, at least 25, at least 30, at least 40, at least 50, at least 75, at least 100, at least 150, at least 200, at least 300, at least 400, at least 500, at least 750, or at least 1000 consecutive nucleotides of one or more of SEQ ID NOs: 1 and 50, and where the mutant allele of the endogenous br2 locus produces a RNA transcript comprising the antisense RNA sequence. In another aspect, a method further comprises regenerating or developing a corn plant from the corn cell.

In an aspect, a targeted editing technique used here comprises the use of at least one site-specific nuclease. In an aspect, a site-specific nuclease is selected from the group consisting of a zinc-finger nuclease, a meganuclease, an RNA-guided nuclease, a TALEN, a recombinase, a transposase, and any combination thereof. In another aspect, a site-specific nuclease is a RNA-guided nuclease selected from the group consisting of a Cas9 nuclease or a variant thereof, and a Cpf1 nuclease or a variant thereof.

In an aspect, a method provided here inserts into the endogenous br2 locus a DNA segment originating from the endogenous br2 locus. In another aspect, an inserted DNA segment is provided in a donor template. In a further aspect, an inserted DNA segment is provided by excising the DNA segment from another chromosomal location (e.g., trans-fragment template).

According to further embodiments, methods are provided for transforming a plant cell, tissue or explant with a recombinant DNA molecule or construct encoding one or more molecules required for targeted genome editing (e.g., guide RNAs or site-directed nucleases). Numerous methods for transforming chromosomes or plastids in a plant cell with a recombinant DNA molecule or construct are known in the art, which may be used according to method embodiments of the present invention to produce a transgenic plant cell and plant. Any suitable method or technique for transformation of a plant cell known in the art may be used according to present methods. Effective methods for transformation of plants include bacterially mediated transformation, such as Agrobacterium-mediated or Rhizobium-mediated transformation, and microprojectile or particle bombardment-mediated transformation. A variety of methods are known in the art for transforming explants with a transformation vector via bacterially mediated transformation or microprojectile or particle bombardment and then subsequently culturing, etc., those explants to regenerate or develop transgenic plants. Other methods for plant transformation, such as microinjection, electroporation, vacuum infiltration, pressure, sonication, silicon carbide fiber agitation, PEG-mediated transformation, etc., are also known in the art.

Methods of transforming plant cells and explants are well known by persons of ordinary skill in the art. Methods for transforming plant cells by microprojectile bombardment with particles coated with recombinant DNA are provided, for example, in U.S. Pat. Nos. 5,550,318; 5,538,880 6,160,208; 6,399,861; and 6,153,812, and Agrobacterium-mediated transformation is described, for example, in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any suitable method of plant transformation known or later developed in the art can be used to transform a plant cell or explant with any of the nucleic acid molecules, constructs or vectors provided herein.

Recipient cell(s) or explant or cellular targets for transformation include, but are not limited to, a seed cell, a fruit cell, a leaf cell, a cotyledon cell, a hypocotyl cell, a meristem cell, an embryo cell, an endosperm cell, a root cell, a shoot cell, a stem cell, a pod cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, a phloem cell, a bud cell, a callus cell, a chloroplast, a stomatal cell, a trichome cell, a root hair cell, a storage root cell, or a vascular tissue cell, a seed, embryo, meristem, cotyledon, hypocotyl, endosperm, root, shoot, stem, node, callus, cell suspension, protoplast, flower, leaf, pollen, anther, ovary, ovule, pericarp, bud, and/or vascular tissue, or any transformable portion of any of the foregoing. For plant transformation, any target cell(s), tissue(s), explant(s), etc., that may be used to receive a recombinant DNA transformation vector or molecule of the present disclosure may be collectively be referred to as an “explant” for transformation. Preferably, a transformable or transformed explant cell or tissue may be further developed or regenerated into a plant. Any cell or explant from which a fertile plant can be grown or regenerated is contemplated as a useful recipient cell or explant for practice of this disclosure (i.e., as a target explant for transformation). Callus can be initiated or created from various tissue sources, including, but not limited to, embryos or parts of embryos, non-embryonic seed tissues, seedling apical meristems, microspores, and the like. Any cells that are capable of proliferating as callus may serve as recipient cells for transformation. Transformation methods and materials for making transgenic plants (e.g., various media and recipient target cells or explants and methods of transformation and subsequent regeneration of into transgenic plants) are known in the art.

Transformation or editing of a target plant material or explant may be practiced in tissue culture on nutrient media, for example a mixture of nutrients that allow cells to grow in vitro or cell culture. Modified explants, cells or tissues may be subjected to additional culturing steps, such as callus induction, selection, regeneration, etc., as known in the art. Transformation or editing may also be carried out without creation or use of a callus tissue. Transformed or edited cells, tissues or explants containing a DNA sequence insertion or edit may be grown, developed or regenerated into transgenic plants in culture, plugs, or soil according to methods known in the art. Modified plants may be further crossed to themselves or other plants to produce modified plant seeds and progeny. A modified plant may also be prepared by crossing a first plant comprising a DNA sequence or construct or an edit (e.g., an antisense sequence, deletion, or inversion) with a second plant lacking the insertion. For example, a DNA sequence, deletion, antisense sequence or inversion may be introduced into a first plant line that is amenable to transformation or editing, which may then be crossed with a second plant line to introgress the DNA sequence or edit (e.g., an antisense sequence, deletion, or inversion) into the second plant line. Progeny of these crosses can be further back crossed into the desirable line multiple times, such as through 6 to 8 generations or back crosses, to produce a progeny plant with substantially the same genotype as the original parental line, but for the introduction of the DNA sequence or edit.

A transgenic or modified plant, plant part, cell, or explant provided herein may be of an elite variety or an elite line. An elite variety or an elite line refers to a variety that has resulted from breeding and selection for superior agronomic performance. A transgenic or edited plant, cell, or explant provided herein may be a hybrid plant, cell, or explant. As used herein, a “hybrid” is created by crossing two plants from different varieties, lines, inbreds, or species, such that the progeny comprises genetic material from each parent. Skilled artisans recognize that higher order hybrids can be generated as well. For example, a first hybrid can be made by crossing Variety A with Variety B to create an A x B hybrid, and a second hybrid can be made by crossing Variety C with Variety D to create a C x D hybrid. The first and second hybrids can be further crossed to create the higher order hybrid (A×B)×(C×D) comprising genetic information from all four parent varieties.

In an aspect, this disclosure provides a method for generating a corn plant comprising: (a) fertilizing at least one female corn plant with pollen from a male corn plant, where the female corn plant comprises a mutant allele of an endogenous Brachytic2 (br2) locus, where the mutant allele comprises a DNA segment inserted into the endogenous br2 locus, where the DNA segment encodes an antisense RNA sequence that is at least 70%, at least 75%, at least 80%, at least 85%, at least 90%, at least 95%, at least 96%, at least 97%, at least 98%, at least 99%, or 100% complementary to at least 20, at least 25, at least 30, at least 40, at least 50, at least 75, at least 100, at least 150, at least 200, at least 300, at least 400, at least 500, at least 750, or at least 1000 consecutive nucleotides of one or more of SEQ ID NOs: 1 and 50, and where the mutant allele of the endogenous br2 locus produces a RNA transcript comprising the antisense RNA sequence; and (b) obtaining at least one seed produced by said fertilizing of step (a). In another aspect, the method further comprises (c) growing the at least one seed obtained in step (b) to generate at least one progeny corn plant comprising said mutant allele. In an aspect, the at least one progeny corn plant obtained in step (c) is heterozygous for the mutant allele. In another aspect, the method further comprises (c) growing the at least one seed obtained in step (b) to generate at least one progeny corn plant comprising said mutant allele. In an aspect, the at least one progeny corn plant obtained in step (c) is homozygous or biallelic for the mutant allele.

In an aspect, the female corn plant is homozygous for a mutant allele. In another aspect, the female corn plant is heterozygous for the mutant allele. In another aspect, the female corn plant is biallelic for a first mutant allele and a second mutant allele. In an aspect, the male corn plant lacks the mutant allele. In an aspect, the male corn plant is heterozygous for the mutant allele. In an aspect, the male corn plant is homozygous for the mutant allele. In an aspect, the male corn plant is biallelic for a first mutant allele and a second mutant allele. In an aspect, the at least one progeny corn plant has a shorter plant height and/or improved lodging resistance relative to an control plant lacking the mutant allele. In an aspect, the at least one progeny corn plant has a shorter plant height and/or improved lodging resistance relative to the male corn plant. In an aspect, the female corn plant is an inbred corn plant. In an aspect, the female corn plant is a hybrid corn plant. In an aspect, the male corn plant is an inbred corn plant. In an aspect, the male corn plant is a hybrid corn plant. In an aspect, the female corn plant is an elite corn plant. In an aspect, the male corn plant is an elite corn plant. In an aspect, the female corn plant is of a first inbred corn line or variety, and the male corn plant is of a different, second inbred corn line or variety. In an aspect, the female corn plant and the male corn plant are grown in a greenhouse or growth chamber. In an aspect, the female corn plant and the male corn plant are grown outdoors. In an aspect, the female corn plant has been detasseled. In an aspect, the female corn plant is a cytoplasmically male sterile corn plant.

As used herein, “detasseled” corn refers to corn where the pollen-producing flowers, or tassels, have been removed. Detasseling is typically performed before the tassel can shed pollen. Detasseling can be accomplished via machine detasseling, manual detasseling, or a combination of both machine and manual detasseling. Detasseling often removes the uppermost leaves of the corn plant along with the developing tassel. Detasseled corn plants retain their female flowers, which eventually produce kernels on the ear. In an aspect, a corn plant provided herein is a detasseled corn plant. As an alternative to chemical treatment, corn plants (or female corn plants) can be made male sterile through genetic crosses and inheritance causing cytoplasmic male sterility. As used herein, the term “cytoplasmic male sterility” or “CMS” refers to a condition where a corn plant is partially or fully incapable of producing functional pollen. As known in the art, cytoplasmic male sterility is a maternally inherited trait that is commonly associated with unusual open reading frames within the mitochondrial genome which cause cytoplasmic dysfunction. In an aspect, a corn plant or female corn plant provided herein is a cytoplasmic male sterile corn plant.

A plant selectable marker transgene in a transformation vector or construct of the present disclosure may be used to assist in the selection of transformed cells or tissue due to the presence of a selection agent, such as an antibiotic or herbicide, wherein the plant selectable marker transgene provides tolerance or resistance to the selection agent. Thus, the selection agent may bias or favor the survival, development, growth, proliferation, etc., of transformed cells expressing the plant selectable marker gene, such as to increase the proportion of transformed cells or tissues in the R₀ plant. Commonly used plant selectable marker genes include, for example, those conferring tolerance or resistance to antibiotics, such as kanamycin and paromomycin (nptII), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (aroA or EPSPS). Plant screenable marker genes may also be used, which provide an ability to visually screen for transformants, such as luciferase or green fluorescent protein (GFP), or a gene expressing a beta glucuronidase or uidA gene (GUS) for which various chromogenic substrates are known. In some embodiments, a vector or polynucleotide provided herein comprises at least one selectable marker gene selected from the group consisting of nptII, aph IV, aad4, aac3, aacC4, bar, pat, DMO, EPSPS, aroA, GFP, and GUS. Plant transformation may also be carried out in the absence of selection during one or more steps or stages of culturing, developing or regenerating transformed explants, tissues, plants and/or plant parts.

According to present embodiments, methods for transforming a plant cell, tissue or explant with a recombinant DNA molecule or construct may further include site-directed or targeted integration. According to these methods, a portion of a recombinant DNA donor template molecule (i.e., an insertion sequence) may be inserted or integrated at a desired site or locus within the plant genome. The insertion sequence of the donor template may comprise a transgene or construct, such as a transgene or transcribable DNA sequence of interest that encodes an anti-sense RNA sequence that is identical or complementary to an endogenous br2 gene sequence. The donor template may also have one or two homology arms flanking the insertion sequence to promote the targeted insertion event through homologous recombination and/or homology-directed repair. Each homology arm may be at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence within the genome of a monocot or cereal plant (e.g., a corn plant). Thus, a recombinant DNA molecule of the present disclosure may comprise a donor template for site-directed or targeted integration of a transgene or construct, such as a transgene or transcribable DNA sequence of interest that encodes an anti-sense RNA sequence that is identical or complementary to an endogenous br2 gene sequence, into the genome of a plant.

Any site or locus within the genome of a plant may potentially be chosen for site-directed integration of a transgene, construct or transcribable DNA sequence provided herein. For site-directed integration, a double-strand break (DSB) or nick may first be made at a selected genomic locus with a site-specific nuclease, such as, for example, a zinc-finger nuclease (ZFN), an engineered or native meganuclease, a TALE-endonuclease (TALEN), or an RNA-guided endonuclease (e.g., Cas9 or Cpf1). Any method known in the art for site-directed integration may be used. In the presence of a donor template molecule with an insertion sequence, the DSB or nick may then be repaired by homologous recombination between homology arm(s) of the donor template and the plant genome, or by non-homologous end joining (NHEJ), resulting in site-directed integration of the insertion sequence into the plant genome to create the targeted insertion event at the site of the DSB or nick. Thus, site-specific insertion or integration of a transgene, construct or sequence may be achieved.

A site-specific nuclease provided herein may be selected from the group consisting of a zinc-finger nuclease (ZFN), a meganuclease, an RNA-guided endonuclease, a TALEN, a recombinase, a transposase, or any combination thereof. See, e.g., Khandagale, K. et al., “Genome editing for targeted improvement in plants,” Plant Biotechnol Rep 10: 327-343 (2016); and Gaj, T. et al., “ZFN, TALEN and CRISPR/Cas-based methods for genome engineering,” Trends Biotechnol. 31(7): 397-405 (2013), the contents and disclosures of which are incorporated herein by reference. A recombinase may be a serine recombinase attached to a DNA recognition motif, a tyrosine recombinase attached to a DNA recognition motif or other recombinase enzyme known in the art. A recombinase or transposase may be a DNA transposase or recombinase attached to a DNA binding domain. A tyrosine recombinase attached to a DNA recognition motif may be selected from the group consisting of a Cre recombinase, a Flp recombinase, and a Tnp1 recombinase. According to some embodiments, a Cre recombinase or a Gin recombinase provided herein is tethered to a zinc-finger DNA binding domain. In another embodiment, a serine recombinase attached to a DNA recognition motif provided herein is selected from the group consisting of a PhiC31 integrase, an R4 integrase, and a TP-901 integrase. In another embodiment, a DNA transposase attached to a DNA binding domain provided herein is selected from the group consisting of a TALE-piggyBac and TALE-Mutator.

According to embodiments of the present disclosure, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, and homologs or modified versions thereof, Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo) and homologs or modified versions thereof. According to some embodiments, an RNA-guided endonuclease may be a Cas9 or Cpf1 (or Cas12a) enzyme.

In an aspect, a site-specific nuclease provided herein is selected from the group consisting of a zinc-finger nuclease, a meganuclease, an RNA-guided nuclease, a TALEN, a recombinase, a transposase, or any combination thereof. In another aspect, a site-specific nuclease provided herein is selected from the group consisting of a Cas9 or a Cpf1 (or Cas12a). In another aspect, a site-specific nuclease provided herein is selected from the group consisting of a Cas1, a Cas1B, a Cas2, a Cas3, a Cas4, a Cas5, a Cas6, a Cas7, a Cas8, a Cas9, a Cas10, a Csy1, a Csy2, a Csy3, a Cse1, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx15, a Csf1, a Csf2, a Csf3, a Csf4, a Cpf1, CasX, CasY, a homolog thereof, or a modified version thereof. In another aspect, an RNA-guided nuclease provided herein is selected from the group consisting of a Cas9 or a Cpf1 (or Cas12a). In another aspect, an RNA guided nuclease provided herein is selected from the group consisting of a Cas1, a Cas1B, a Cas2, a Cas3, a Cas4, a Cas5, a Cas6, a Cas7, a Cas8, a Cas9, a Cas10, a Csy1, a Csy2, a Csy3, a Cse1, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx15, a Csf1, a Csf2, a Csf3, a Csf4, a Cpf1, CasX, CasY, a homolog thereof, or a modified version thereof. In another aspect, a method and/or a composition provided herein comprises at least one, at least two, at least three, at least four, at least five, at least six, at least seven, at least eight, at least nine, or at least ten site-specific nucleases. In yet another aspect, a method and/or a composition provided herein comprises at least one, at least two, at least three, at least four, at least five, at least six, at least seven, at least eight, at least nine, or at least ten polynucleotides encoding at least one, at least two, at least three, at least four, at least five, at least six, at least seven, at least eight, at least nine, or at least ten site-specific nucleases.

For RNA-guided endonucleases, a guide RNA (gRNA) molecule is further provided to direct the endonuclease to a target site in the genome of the plant via base-pairing or hybridization to cause a DSB or nick at or near the target site. The gRNA may be transformed or introduced into a plant cell or tissue (perhaps along with a nuclease, or nuclease-encoding DNA molecule, construct or vector) as a gRNA molecule, or as a recombinant DNA molecule, construct or vector comprising a transcribable DNA sequence encoding the guide RNA operably linked to a plant-expressible promoter. As understood in the art, a “guide RNA” may comprise, for example, a CRISPR RNA (crRNA), a single-chain guide RNA (sgRNA), or any other RNA molecule that may guide or direct an endonuclease to a specific target site in the genome. A “single-chain guide RNA” (or “sgRNA”) is a RNA molecule comprising a crRNA covalently linked a tracrRNA by a linker sequence, which may be expressed as a single RNA transcript or molecule. The guide RNA comprises a guide or targeting sequence that is identical or complementary to a target site within the plant genome, such as at or near a br2 gene. A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu, X. et al., “Target specificity of the CRISPR-Cas9 system,” Quant Biol. 2(2): 59-70 (2014), the content and disclosure of which is incorporated herein by reference. The genomic PAM sequence on the sense (+) strand adjacent to the target site (relative to the targeting sequence of the guide RNA) may comprise 5′-NGG-3′. However, the corresponding sequence of the guide RNA (i.e., immediately downstream (3′) to the targeting sequence of the guide RNA) may generally not be complementary to the genomic PAM sequence. The guide RNA may typically be a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

According to some embodiments, a recombinant DNA construct or vector may comprise a first polynucleotide sequence encoding a site-specific nuclease and a second polynucleotide sequence encoding a guide RNA that may be introduced into a plant cell together via plant transformation techniques. Alternatively, two recombinant DNA constructs or vectors may be provided including a first recombinant DNA construct or vector and a second DNA construct or vector that may be introduced into a plant cell together or sequentially via plant transformation techniques, wherein the first recombinant DNA construct or vector comprises a polynucleotide sequence encoding a site-specific nuclease and the second recombinant DNA construct or vector comprises a polynucleotide sequence encoding a guide RNA. According to some embodiments, a recombinant DNA construct or vector comprising a polynucleotide sequence encoding a site-specific nuclease may be introduced via plant transformation techniques into a plant cell that already comprises (or is transformed with) a recombinant DNA construct or vector comprising a polynucleotide sequence encoding a guide RNA. Alternatively, a recombinant DNA construct or vector comprising a polynucleotide sequence encoding a guide RNA may be introduced via plant transformation techniques into a plant cell that already comprises (or is transformed with) a recombinant DNA construct or vector comprising a polynucleotide sequence encoding a site-specific nuclease. According to yet further embodiments, a first plant comprising (or transformed with) a recombinant DNA construct or vector comprising a polynucleotide sequence encoding a site-specific nuclease may be crossed with a second plant comprising (or transformed with) a recombinant DNA construct or vector comprising a polynucleotide sequence encoding a guide RNA. Such recombinant DNA constructs or vectors may be transiently transformed into a plant cell or stably transformed or integrated into the genome of a plant cell.

In an aspect, vectors comprising polynucleotides encoding a site-specific nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In an aspect, vectors comprising polynucleotides encoding a Cas9 nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In another aspect, vectors comprising polynucleotides encoding a Cpf1 and, optionally one or more, two or more, three or more, or four or more crRNAs are provided to a cell by transformation methods known in the art (e.g., without being limiting, viral transfection, particle bombardment, PEG-mediated protoplast transfection or Agrobacterium-mediated transformation).

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site. According to many of these embodiments, non-RNA-guided site-specific nucleases, such as recombinases, zinc finger nucleases (ZFNs), meganucleases, and TALENs, may be designed, engineered and constructed according to known methods to target and bind to a target site at or near the genomic locus of an endogenous br2 gene of a corn plant to create a DSB or nick at such genomic locus to knockout or knockdown expression of the br2 gene via repair of the DSB or nick. For example, an engineered site-specific nuclease, such as a recombinase, zinc finger nuclease (ZFN), meganuclease, or TALEN, may be designed to target and bind to a target site within the genome of a plant corresponding to a sequence within SEQ ID NO: 1 or 50, or its complementary sequence, to create a DSB or nick at the genomic locus for the br2 gene, which may then lead to the creation of a mutation or insertion of a sequence at the site of the DSB or nick, through cellular repair mechanisms, which may be guided by a donor molecule or template.

In an aspect, a targeted genome editing technique described herein may comprise the use of a recombinase. In some embodiments, a tyrosine recombinase attached, etc., to a DNA recognition domain or motif may be selected from the group consisting of a Cre recombinase, a Flp recombinase, and a Tnp1 recombinase. In an aspect, a Cre recombinase or a Gin recombinase provided herein may be tethered to a zinc-finger DNA binding domain. The Flp-FRTsite-directed recombination system may come from the 2p plasmid from the baker's yeast Saccharomyces cerevisiae. In this system, Flp recombinase (flippase) may recombine sequences between flippase recognition target (FRT) sites. FRT sites comprise 34 nucleotides. Flp may bind to the “arms” of the FRT sites (one arm is in reverse orientation) and cleaves the FRT site at either end of an intervening nucleic acid sequence. After cleavage, Flp may recombine nucleic acid sequences between two FRT sites. Cre-lox is a site-directed recombination system derived from the bacteriophage P1 that is similar to the Flp-FRT recombination system. Cre-lox can be used to invert a nucleic acid sequence, delete a nucleic acid sequence, or translocate a nucleic acid sequence. In this system, Cre recombinase may recombine a pair of lox nucleic acid sequences. Lox sites comprise 34 nucleotides, with the first and last 13 nucleotides (arms) being palindromic. During recombination, Cre recombinase protein binds to two lox sites on different nucleic acids and cleaves at the lox sites. The cleaved nucleic acids are spliced together (reciprocally translocated) and recombination is complete. In another aspect, a lox site provided herein is a loxP, lox 2272, loxN, lox 511, lox 5171, lox71, lox66, M2, M3, M7, or M11 site.

ZFNs are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The DNA-binding domain of a ZFN may typically be composed of 3-4 (or more) zinc-fingers. The amino acids at positions −1, +2, +3, and +6 relative to the start of the zinc finger α-helix, which contribute to site-specific binding to the target site, can be changed and customized to fit specific target sequences. The other amino acids may form a consensus backbone to generate ZFNs with different sequence specificities. Methods and rules for designing ZFNs for targeting and binding to specific target sequences are known in the art. See, e.g., US Patent App. Nos. 2005/0064474, 2009/0117617, and 2012/0142062, the contents and disclosures of which are incorporated herein by reference. The FokI nuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site.

Without being limited by any scientific theory, because the DNA-binding specificities of zinc finger domains can be re-engineered using one of various methods, customized ZFNs can theoretically be constructed to target nearly any target sequence (e.g., at or near a br2 gene in a plant genome). Publicly available methods for engineering zinc finger domains include Context-dependent Assembly (CoDA), Oligomerized Pool Engineering (OPEN), and Modular Assembly. In an aspect, a method and/or composition provided herein comprises one or more, two or more, three or more, four or more, or five or more ZFNs. In another aspect, a ZFN provided herein is capable of generating a targeted DSB or nick. In an aspect, vectors comprising polynucleotides encoding one or more, two or more, three or more, four or more, or five or more ZFNs are provided to a cell by transformation methods known in the art (e.g., without being limiting, viral transfection, particle bombardment, PEG-mediated protoplast transfection, or Agrobacterium-mediated transformation). The ZFNs may be introduced as ZFN proteins, as polynucleotides encoding ZFN proteins, and/or as combinations of proteins and protein-encoding polynucleotides.

Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). According to some embodiments, a meganuclease may comprise a scaffold or base enzyme selected from the group consisting of I-CreI, I-CeuI, I-MsoI, I-SeI, I-AniI, and I-DmoI. The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity. Thus, a meganuclease may be selected or engineered to bind to a genomic target sequence in a plant, such as at or near the genomic locus of a br2 gene. In an aspect, a method and/or composition provided herein comprises one or more, two or more, three or more, four or more, or five or more meganucleases. In another aspect, a meganuclease provided herein is capable of generating a targeted DSB. In an aspect, vectors comprising polynucleotides encoding one or more, two or more, three or more, four or more, or five or more meganucleases are provided to a cell by transformation methods known in the art (e.g., without being limiting, viral transfection, particle bombardment, PEG-mediated protoplast transfection or Agrobacterium-mediated transformation).

TALENs are artificial restriction enzymes generated by fusing the transcription activator-like effector (TALE) DNA binding domain to a nuclease domain (e.g., FokI). When each member of a TALEN pair binds to the DNA sites flanking a target site, the FokI monomers dimerize and cause a double-stranded DNA break at the target site. Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity.

TALENs are artificial restriction enzymes generated by fusing the transcription activator-like effector (TALE) DNA binding domain to a nuclease domain. In some aspects, the nuclease is selected from a group consisting of PvuII, MutH, TevI, FokI, Ahwi, MlyI, SbfI, SdaI, StsI, CIeDORF, Clo051, and Pep1071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN is also refers to one or both members of a pair of TALENs that work together to cleave DNA at the same site.

Transcription activator-like effectors (TALEs) can be engineered to bind practically any DNA sequence, such as at or near the genomic locus of a br2 gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al. 2013. PLoS One. 8: e82539). MutH is capable of introducing strand-specific nicks in DNA (see Gabsalilow et al. 2013. Nucleic Acids Research. 41: e83). TevI introduces double-stranded breaks in DNA at targeted sites (see Beurdeley et al., 2013. Nature Communications. 4: 1762).

The relationship between amino acid sequence and DNA recognition of the TALE binding domain allows for designable proteins. Software programs such as DNA Works can be used to design TALE constructs. Other methods of designing TALE constructs are known to those of skill in the art. See Doyle et al., Nucleic Acids Research (2012) 40: W117-122.; Cermak et al., Nucleic Acids Research (2011). 39:e82; and tale-nt.cac.cornell.edu/about. In an aspect, a method and/or composition provided herein comprises one or more, two or more, three or more, four or more, or five or more TALENs. In another aspect, a TALEN provided herein is capable of generating a targeted DSB. In an aspect, vectors comprising polynucleotides encoding one or more, two or more, three or more, four or more, or five or more TALENs are provided to a cell by transformation methods known in the art (e.g., without being limiting, viral transfection, particle bombardment, PEG-mediated protoplast transfection or Agrobacterium-mediated transformation). See, e.g., US Patent App. Nos. 2011/0145940, 2011/0301073, and 2013/0117869, the contents and disclosures of which are incorporated herein by reference.

For purposes of the present disclosure, a “plant” includes an explant, plant part, seedling, plantlet or whole plant at any stage of regeneration or development. As used herein, a “plant part” may refer to any organ or intact tissue of a plant, such as a meristem, shoot organ/structure (e.g., leaf, stem or node), root, flower or floral organ/structure (e.g., bract, sepal, petal, stamen, carpel, anther and ovule), seed (e.g., embryo, endosperm, and seed coat), fruit (e.g., the mature ovary), propagule, or other plant tissues (e.g., vascular tissue, dermal tissue, ground tissue, and the like), or any portion thereof. Plant parts of the present disclosure may be viable, nonviable, regenerable, and/or non-regenerable. A “propagule” may include any plant part that can grow into an entire plant.

Embodiments of the present disclosure further include methods for making or producing transgenic or modified plants described here, such as by transformation, genome editing, mutating, crossing, etc., wherein the method comprises introducing a recombinant DNA molecule, construct or sequence of interest into a plant cell, or editing or mutating the genomic locus of an endogenous br2 gene, and then regenerating or developing the transgenic or modified plant from the transformed or edited plant cell, which may be performed under selection pressure. Such methods may comprise transforming a plant cell with a recombinant DNA molecule, construct or sequence of interest, and selecting for a plant having one or more altered phenotypes or traits, such as one or more of the following traits at one or more stages of development: shorter or semi-dwarf stature or plant height, shorter internode length in one or more internode(s), increased stalk/stem diameter, improved lodging resistance, reduced green snap, deeper roots, increased leaf area, earlier canopy closure, increased foliar water content and/or higher stomatal conductance under water limiting conditions, reduced anthocyanin content and/or area in leaves under normal or nitrogen or water limiting stress conditions, improved yield-related traits including a larger female reproductive organ or ear, an increase in ear weight, harvest index, yield, seed or kernel number, and/or seed or kernel weight, increased stress tolerance, such as increased drought tolerance, increased nitrogen utilization, and/or increased tolerance to high density planting, as compared to a wild type or control plant.

According to another aspect of the present disclosure, methods are provided for planting a modified or transgenic plant(s) provided herein at a normal/standard or high density in field. According to some embodiments, the yield of a crop plant per acre (or per land area) may be increased by planting a modified or transgenic plant(s) of the present disclosure at a higher density in the field. As described herein, modified or transgenic plants having a genome-edited br2 gene, may have reduced plant height, shorter internode(s), increased stalk/stem diameter, and/or increased lodging resistance. It is proposed that modified or transgenic plants may tolerate high density planting conditions since an increase in stem diameter may resist lodging and the shorter plant height may allow for increased light penetrance to the lower leaves under high density planting conditions. Thus, modified or transgenic plants provided herein may be planted at a higher density to increase the yield per acre (or land area) in the field. For row crops, higher density may be achieved by planting a greater number of seeds/plants per row length and/or by decreasing the spacing between rows.

According to some embodiments, a modified or transgenic crop plant may be planted at a density in the field (plants per land/field area) that is at least 5%, 10%, 15%, 20%, 25%, 50%, 75%, 100%, 125%, 150%, 175%, 200%, 225%, or 250% higher than the normal planting density for that crop plant according to standard agronomic practices. A modified or transgenic crop plant may be planted at a density in the field of at least 38,000 plants per acre, at least 40,000 plants per acre, at least 42,000 plants per acre, at least 44,000 plants per acre, at least 45,000 plants per acre, at least 46,000 plants per acre, at least 48,000 plants per acre, 50,000 plants per acre, at least 52,000 plants per acre, at least 54,000 per acre, or at least 56,000 plants per acre. As an example, corn plants may be planted at a higher density, such as in a range from about 38,000 plants per acre to about 60,000 plants per acre, or about 40,000 plants per acre to about 58,000 plants per acre, or about 42,000 plants per acre to about 58,000 plants per acre, or about 40,000 plants per acre to about 45,000 plants per acre, or about 45,000 plants per acre to about 50,000 plants per acre, or about 50,000 plants per acre to about 58,000 plants per acre, or about 52,000 plants per acre to about 56,000 plants per acre, or about 38,000 plants per acre, about 42,000 plant per acre, about 46,000 plant per acre, or about 48,000 plants per acre, about 50,000 plants per acre, or about 52,000 plants per acre, or about 54,000 plant per acre, as opposed to a standard density range, such as about 18,000 plants per acre to about 38,000 plants per acre.

Corn leaves consist of four main anatomical parts: a proximal sheath, a ligule, an auricle, and a distal blade. The sheath wraps around the stem and younger leaves, while the blade is flattened in the mediolateral axis (midrib to margin). The ligule and auricle are found at the blade/sheath boundary; the ligule is an adaxial (upper) membranous structure that acts as a collar around the stem, and the auricle is a projection on the lower surface of the blade base that connects the blade to the sheath. Stages of corn plant growth are divided into vegetative (V) stages and reproductive (R) stages. Upon germination, a corn plant is in the VE stage (emergence). Once the first leaf collar (e.g., the ligule) is visible, the corn plant is in the VI stage. The emergence of the second leaf collar signifies V2 stage; the emergence of the third leaf collar signifies the V3 stage; and so on until the tassel emerges. For example, if twelve leaf collars are visible, the plant is a V12 stage plant. Once the bottom-most branch of the tassel emerges the plant is in VT stage, which is the final vegetative stage. The reproductive stage of growth occurs after the vegetative stage. The number of vegetative stages prior to VT stage can vary by environment and corn line. The first reproductive stage (R1; silking stage) occurs when silk is visible outside the husk leaves surrounding an ear of corn. R2 (blistering stage) occurs when corn kernels are white on the outside and are filled with a clear liquid inside. R3 (milk stage) occurs when the kernels are yellow on the outside and are filled with a milky white fluid inside. R4 (dough stage) occurs when the kernels are filled with a thick, or pasty, fluid. In some corn lines the cob will also turn pink or red at this stage. R5 (dent stage) occurs when a majority of the kernels are at least partially dented. The final reproductive stage, R6 (physiological maturity), occurs when the kernels have attained their maximum dry weight.

The height of a corn plant can be measured using a variety of methods known in the art. The height of a corn plant can also be determined based on a variety of anatomical locations on a corn plant. In an aspect, the height of a corn plant is measured as the distance between the soil or ground and the ligule of the uppermost fully-expanded leaf of the corn plant. As used herein, a “fully-expanded leaf” is a leaf where the leaf blade is exposed and both the ligule and auricle are visible at the blade/sheath boundary. In another aspect, the height of a corn plant is measured as the distance between the soil or ground and the upper leaf surface of the leaf farthest from the soil. In another aspect, the height of a corn plant is measured as the distance between the soil or ground and the arch of the highest corn leaf that is at least 50% developed. As used herein, an “arch of the highest corn leaf” is the highest point of the arch of the uppermost leaf of the corn plant that is curving downward. In another aspect, the height of a corn plant is measured at the first reproductive (R1) stage. Exemplary, non-limiting methods of measuring plant height include comparing photographs of corn plants to a height reference, or physically measuring individual corn plants with a suitable ruler. Unless otherwise specified, corn plant heights are measured at RI stage. Those in the art recognize that, when comparing a modified corn plant to a control corn plant, the measurements must be made at the same stage of growth. It would be improper, as a non-limiting example, to compare the height of a modified corn plant at R3 stage to the height of a control corn plant at V6 stage, even if both plants had been growing for the same amount of time.

As used herein, the term “ground” or “ground level” used in relation to a corn plant, such as to measure plant height, refers to the top or uppermost surface of the growth medium or soil (e.g., earth) from which the corn plant grows.

Corn plant height varies depending on the line or variety grown, whether the plant is a hybrid or inbred, and environmental conditions. Although hybrid corn plants can reach a height of over 3.6 meters tall by maturity, a height of around 2.0-2.5 meters by maturity for hybrid plants is more common.

According to embodiments of the present disclosure, a modified corn plant(s) is/are provided that comprise (i) a plant height of less than 2000 mm, less than 1950 mm, less than 1900 mm, less than 1850 mm, less than 1800 mm, less than 1750 mm, less than 1700 mm, less than 1650 mm, less than 1600 mm, less than 1550 mm, less than 1500 mm, less than 1450 mm, less than 1400 mm, less than 1350 mm, less than 1300 mm, less than 1250 mm, less than 1200 mm, less than 1150 mm, less than 1100 mm, less than 1050 mm, or less than 1000 mm, and/or (ii) an ear height of at least 500 mm, at least 600 mm, at least 700 mm, at least 800 mm, at least 900 mm, at least 1000 mm, at least 1100 mm, at least 1200 mm, at least 1300 mm, at least 1400 mm, or at least 1500 mm. Any such plant height trait or range that is expressed in millimeters (mm) may be converted into a different unit of measurement based on known conversions (e.g., one inch is equal to 2.54 cm or 25.4 millimeters, and millimeters (mm), centimeters (cm) and meters (m) only differ by one or more powers of ten). Thus, any measurement provided herein is further described in terms of any other comparable units of measurement according to known and established conversions. However, the exact plant height and/or ear height of a modified corn plant may depend on the environment and genetic background. Thus, the change in plant height and/or ear height of a modified corn plant may instead be described in terms of a minimum difference or percent change relative to a control plant. A modified corn plant may further comprise at least one ear that is substantially free of male reproductive tissues or structures or other off-types.

According to embodiments of the present disclosure, modified corn plants are provided that comprise a plant height during late vegetative and/or reproductive stages of development (e.g., at R3 stage) of between 1000 mm and 1800 mm, between 1000 mm and 1700 mm, between 1050 mm and 1700 mm, between 1100 mm and 1700 mm, between 1150 mm and 1700 mm, between 1200 mm and 1700 mm, between 1250 mm and 1700 mm, between 1300 mm and 1700 mm, between 1350 mm and 1700 mm, between 1400 mm and 1700 mm, between 1450 mm and 1700 mm, between 1000 mm and 1500 mm, between 1050 mm and 1500 mm, between 1100 mm and 1500 mm, between 1150 mm and 1500 mm, between 1200 mm and 1500 mm, between 1250 mm and 1500 mm, between 1300 mm and 1500 mm, between 1350 mm and 1500 mm, between 1400 mm and 1500 mm, between 1450 mm and 1500 mm, between 1000 mm and 1600 mm, between 1100 mm and 1600 mm, between 1200 mm and 1600 mm, between 1300 mm and 1600 mm, between 1350 mm and 1600 mm, between 1400 mm and 1600 mm, between 1450 mm and 1600 mm, of between 1000 mm and 2000 mm, between 1200 mm and 2000 mm, between 1200 mm and 1800 mm, between 1300 mm and 1700 mm, between 1400 mm and 1700 mm, between 1400 mm and 1600 mm, between 1400 mm and 1700 mm, between 1400 mm and 1800 mm, between 1400 mm and 1900 mm, between 1400 mm and 2000 mm, or between 1200 mm and 2500 mm, and/or an ear height of between 500 mm and 1500 mm, between 600 mm and 1500 mm, between 700 and 1500 mm, between 800 mm and 1500 mm, between 900 mm and 1500 mm, between 1000 mm and 1500 mm, between 1100 mm and 1500 mm, between 1200 mm and 1500 mm, between 1300 mm and 1500 mm, between 1400 mm and 1500 mm, between 500 mm and 1200 mm, between 500 mm and 1000 mm, between 500 mm and 800 mm, between 500 mm and 600 mm, between 600 mm and 1200 mm, between 600 mm and 1000 mm, or between 600 mm and 800 mm. A modified corn plant may be substantially free of off-types, such as male reproductive tissues or structures in one or more ears of the modified corn plant.

According to embodiments of the present disclosure, modified corn plants are provided that have (i) a plant height that is at least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, or at least 75% less than the height of a wild-type or control plant, and/or (ii) an ear height that is within at least 5%, at least 10%, at least 15%, at least 20%, at least 25%, at least 30%, at least 35%, at least 40%, at least 45%, at least 50% of the ear height of the wild-type or control plant. According to embodiments of the present disclosure, a modified corn plant may have a reduced plant height that is no more than 20%, 25%, 30%, 35%, 40%, 45%, 50%, 55%, or 60% shorter than the height of a wild-type or control plant, and/or an ear height that is within 10%, 15%, 20%, 25%, 30%, 35%, 40%, 45%, or 50% of the ear height of a wild-type or control plant. For example, a modified plant may have (i) a plant height that is at least 10%, at least 15%, or at least 20% less or shorter (i.e., greater than or equal to 10%, 15%, or 20% shorter), but not greater or more than 50% a shorter, than a wild type or control plant, and/or (ii) an ear height that is within 5%, 10%, or 15% than a wild type or control plant. For clarity, the phrases “at least 20% shorter” and “greater than or equal to 20% shorter” would exclude, for example, 10% shorter. For clarity, the phrases “not greater than 50% shorter”, “no more than 50% shorter” and “not more than 50% shorter” would exclude 60% shorter; the phrase “at least 5% greater” would exclude 2% greater; and the phrases “not more than 30% greater” and “no more than 30% greater” would exclude 40% greater.

According to embodiments of the present disclosure, modified corn plants are provided that comprise a height between 2.5% and 75%, between 2.5% and 50%, between 2.5% and 40%, between 2.5% and 30%, between 2.5% and 25%, between 2.5% and 20%, between 2.5% and 15%, between 2.5% and 12.5%, between 2.5% and 10%, between 2.5% and 7.5%, between 2.5% and 5%, between 5% and 75%, between 5% and 50%, between 10% and 70%, between 10% and 65%, between 10% and 60%, between 10% and 55%, between 10% and 50%, between 10% and 45%, between 10% and 40%, between 10% and 35%, between 10% and 30%, between 10% and 25%, between 10% and 20%, between 10% and 15%, between 10% and 10%, between 10% and 75%, between 25% and 75%, between 10% and 50%, between 20% and 50%, between 25% and 50%, between 30% and 75%, between 30% and 50%, between 25% and 50%, between 15% and 50%, between 20% and 50%, between 25% and 45%, or between 30% and 45% less than the height of a wild-type or control plant, and/or an ear height that is within between 5% and 50%, between 5% and 45%, between 5% and 40%, between 5% and 35%, between 5% and 30%, between 5% and 25%, between 5% and 20%, between 5% and 15%, between 5% and 10%, between 10% and 50%, between 10% and 45%, between 40% and 35%, between 10% and 30%, between 10% and 25%, between 10% and 20%, between 10% and 15%, between 25% and 45%, between 25% and 40%, between 20% and 50%, between 8% and 20%, or between 8% and 15% of the ear height of the wild-type or control plant.

As used herein, “internode length” refers to the distance between two consecutive internodes on the stem of a plant. According to embodiments of the present disclosure, modified corn plants are provided that comprise an average internode length (or a minus-2 internode length and/or minus-4 internode length relative to the position of the ear) that is at least 5%, at least 10%, at least 15%, at least 20%, at least 25%, at least 30%, at least 35%, at least 40%, at least 45%, at least 50%, at least 55%, at least 60%, at least 65%, at least 70%, or at least 75% less than the same or average internode length of a wild-type or control plant. The “minus-2 internode” of a corn plant refers to the second internode below the ear of the plant, and the “minus-4 internode” of a corn plant refers to the fourth internode below the ear of the plant According to many embodiments, modified corn plants are provided that have an average internode length (or a minus-2 internode length and/or minus-4 internode length relative to the position of the ear) that is between 5% and 75%, between 5% and 50%, between 10% and 70%, between 10% and 65%, between 10% and 60%, between 10% and 55%, between 10% and 50%, between 10% and 45%, between 10% and 40%, between 10% and 35%, between 10% and 30%, between 10% and 25%, between 10% and 20%, between 10% and 15%, between 10% and 10%, between 10% and 75%, between 25% and 75%, between 10% and 50%, between 20% and 50%, between 25% and 50%, between 30% and 75%, between 30% and 50%, between 25% and 50%, between 15% and 50%, between 20% and 50%, between 25% and 45%, or between 30% and 45% less than the same or average internode length of a wild-type or control plant.

According to embodiments of the present disclosure, modified corn plants are provided that comprise an ear weight (individually or on average) that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater than the ear weight of a wild-type or control plant. A modified corn plant provided herein may comprise an ear weight that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 25% and 75%, between 25% and 50%, or between 50% and 75% greater than the ear weight of a wild-type or control plant.

According to embodiments of the present disclosure, modified corn plants are provided that have a harvest index of at least 0.57, at least 0.58, at least 0.59, at least 0.60, at least 0.61, at least 0.62, at least 0.63, at least 0.64, or at least 0.65 (or greater). A modified corn plant may comprise a harvest index of between 0.57 and 0.65, between 0.57 and 0.64, between 0.57 and 0.63, between 0.57 and 0.62, between 0.57 and 0.61, between 0.57 and 0.60, between 0.57 and 0.59, between 0.57 and 0.58, between 0.58 and 0.65, between 0.59 and 0.65, or between 0.60 and 0.65. A modified corn plant may have a harvest index that is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, or at least 50% greater than the harvest index of a wild-type or control plant. A modified corn plant may have a harvest index that is between 1% and 45%, between 1% and 40%, between 1% and 35%, between 1% and 30%, between 1% and 25%, between 1% and 20%, between 1% and 15%, between 1% and 14%, between 1% and 13%, between 1% and 12%, between 1% and 11%, between 1% and 10%, between 1% and 9%, between 1% and 8%, between 1% and 7%, between 1% and 6%, between 1% and 5%, between 1% and 4%, between 1% and 3%, between 1% and 2%, between 5% and 15%, between 5% and 20%, between 5% and 30%, or between 5% and 40% greater than the harvest index of a wild-type or control plant.

According to embodiments of the present disclosure, modified corn plants are provided that have an increase in harvestable yield of at least 1 bushel per acre, at least 2 bushels per acre, at least 3 bushels per acre, at least 4 bushels per acre, at least 5 bushels per acre, at least 6 bushels per acre, at least 7 bushels per acre, at least 8 bushels per acre, at least 9 bushels per acre, or at least 10 bushels per acre, relative to a wild-type or control plant. A modified corn plant may have an increase in harvestable yield between 1 and 10, between 1 and 8, between 2 and 8, between 2 and 6, between 2 and 5, between 2.5 and 4.5, or between 3 and 4 bushels per acre. A modified corn plant may have an increase in harvestable yield that is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, or at least 25% greater than the harvestable yield of a wild-type or control plant. A modified corn plant may have a harvestable yield that is between 1% and 25%, between 1% and 20%, between 1% and 15%, between 1% and 14%, between 1% and 13%, between 1% and 12%, between 1% and 11%, between 1% and 10%, between 1% and 9%, between 1% and 8%, between 1% and 7%, between 1% and 6%, between 1% and 5%, between 1% and 4%, between 1% and 3%, between 1% and 2%, between 5% and 15%, between 5% and 20%, between 5% and 25%, between 2% and 10%, between 2% and 9%, between 2% and 8%, between 2% and 7%, between 2% and 6%, between 2% and 5%, or between 2% and 4% greater than the harvestable yield of a wild-type or control plant.

According to embodiments of the present disclosure, a modified corn plant is provided that has a lodging frequency that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% less or lower than a wild-type or control plant. A modified corn plant may have a lodging frequency that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 10% and 40%, between 10% and 30%, between 10% and 20%, between 25% and 75%, between 25% and 50%, or between 50% and 75% less or lower than a wild-type or control plant. Further provided are populations of corn plants having increased lodging resistance and a reduced lodging frequency. Populations of modified corn plants are provided having a lodging frequency that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% less or lower than a population of wild-type or control plants. A population of modified corn plants may comprise a lodging frequency that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 10% and 40%, between 10% and 30%, between 10% and 20%, between 25% and 75%, between 25% and 50%, or between 50% and 75% less or lower than a population of wild-type or control plants, which may be expressed as an average over a specified number of plants or crop area of equal density.

According to embodiments of the present disclosure, modified corn plants are provided having a significantly reduced or decreased plant height (e.g., 2000 mm or less) and a similar ear height, relative to a wild-type or control plant. According to these embodiments, the decrease or reduction in plant height may be within any of the height or percentage ranges recited herein. Such modified corn plants having a reduced plant height relative to a wild-type or control plant may be transformed with a transcribable DNA sequence encoding a non-coding RNA molecule that targets at least one br2 gene for suppression. Modified corn plants having a significantly reduced plant height relative to a wild-type or control plant may further have at least one ear that is substantially free of male reproductive tissues or structures and/or other off-types. Modified corn plants having a significantly reduced plant height relative to a wild-type or control plant may have reduced activity of a br2 gene in one or more tissue(s) of the plant, such as one or more vascular and/or leaf tissue(s) of the plant, relative to the same tissue(s) of the wild-type or control plant. According to many embodiments, modified corn plants may comprise at least one polynucleotide or transcribable DNA sequence encoding a non-coding RNA molecule operably linked to a promoter, which may be a constitutive, tissue-specific or tissue-preferred promoter, wherein the non-coding RNA molecule targets a br2 gene for suppression as provided herein. The non-coding RNA molecule may be a miRNA, siRNA, or miRNA or siRNA precursor molecule. According to some embodiments, modified corn plants having a significantly reduced plant height relative to a wild-type or control plant may further have an increased harvest index and/or increased lodging resistance relative to the wild-type or control plant.

According to embodiments of the present invention, modified corn plants are provided having a reduced gibberellin content (in active form) in at least the stem and internode tissue(s), such as the stem, internode, leaf and/or vascular tissue(s), as compared to the same tissue(s) of wild-type or control plants. According to many embodiments, modified corn plants are provided having a significantly reduced plant height and/or a significantly increased stem diameter relative to wild-type or control plants, wherein the modified corn plants further have significantly reduced or decreased level(s) of one or more auxin or indole-3-acetic acid (IAA) hormones in one or more of the stem, node, internode, leaf and/or vascular tissue(s), relative to the same tissue(s) of the wild-type or control plants. For example, the level of one or more auxins or IAAs in the stem, internode, leaf and/or vascular tissue(s) of a modified corn plant may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% less or lower than in the same tissue(s) of a wild-type or control corn plant.

According to some embodiments, a modified corn plant may comprise levels of one or more auxin or indole-3-acetic acid (IAA) hormones in one or more of the stem, node, internode, leaf and/or vascular tissue(s) that is between 5% and 50%, between 10/a and 100%, between 20% and 100%, between 30% and 100%, between 40% and 100%, between 50% and 100%, between 60% and 100%, between 70% and 100%, between 80% and 100%, between 80% and 90%, between 10% and 90%, between 10% and 80%, between 10% and 70%, between 10% and 60%, between 10% and 50%, between 10% and 40%, between 10% and 30%, between 10% and 20%, between 50% and 100%, between 20% and 90%, between 20% and 80%, between 20% and 70%, between 20% and 60%, between 20% and 50%, between 20% and 40%, between 20% and 40%, between 20% and 30%, between 30% and 90%, between 30% and 80%, between 30% and 70%, between 30% and 60%, between 30% and 50%, between 30% and 40%, between 40% and 90% between 40% and 80%, between 40% and 70%, between 40% and 60%, between 40% and 50%, between 50% and 90%, between 50% and 80%, between 50% and 70%, between 50% and 60%, between 60% and 90%, between 60% and 80%, between 60% and 70%, between 70% and 90%, or between 70% and 80% less or (or lower) than in the same tissue(s) of a wild-type or control corn plant. A modified corn plant having a reduced one or more auxin or indole-3-acetic acid (IAA) hormones in one or more of the stem, node internode, leaf and/or vascular tissue(s), or any portion thereof, may further be substantially free of off-types, such as male reproductive tissues or structures and/or other off-types in at least one ear of a modified corn plant.

According to embodiments of the present disclosure, modified corn plants are provided having a significantly reduced or eliminated expression level of a br2 gene transcript and/or protein in one or more tissue(s), such as one or more stem, internode, leaf and/or vascular tissue(s), of the modified plants, as compared to the same tissue(s) of wild-type or control plants. According to many embodiments, a modified corn plant is provided comprising a significantly reduced plant height relative to wild-type or control plants, wherein the modified corn plant has a significantly reduced or eliminated expression level of a br2 gene transcript(s) and/or protein(s) in one or more tissues, such as one or more stem, internode, leaf and/or vascular tissue(s), of the modified plant, as compared to the same tissue(s) of a wild-type or control corn plant. For example, a modified corn plant has a significantly reduced or eliminated expression level of a br2 gene transcript(s) and/or protein(s), in the whole modified plant, or in one or more stem, internode, leaf and/or vascular tissue(s) of the modified plant, as compared to the same tissue(s) of a wild-type or control plant. For example, the level of a br2 transcript(s) and/or protein(s) in one or more stem, internode, leaf and/or vascular tissue(s) of a modified corn plant may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% less or lower than in the same tissue(s) of a wild-type or control corn plant.

According to some embodiments, a modified corn plant may comprise a level of br2 gene transcript(s) and/or protein(s) in the whole plant, or in one or more stem, node, internode, leaf and/or vascular tissue(s), that is between 5% and 50%, between 10% and 100%, between 20% and 100%, between 30% and 100%, between 40% and 100%, between 50% and 100%, between 60% and 100%, between 70% and 100%, between 80% and 100%, between 80% and 90%, between 10% and 90%, between 10% and 80%, between 10% and 70%, between 10% and 60%, between 10% and 50%, between 10% and 40%, between 10% and 30%, between 10% and 20%, between 50% and 100%, between 20% and 90%, between 20% and 80%, between 20% and 70%, between 20% and 60%, between 20% and 50%, between 20% and 40%, between 20% and 40%, between 20% and 30%, between 30% and 90%, between 30% and 80%, between 30% and 70%, between 30% and 60%, between 30% a and 50%, between 30% and 40%, between 40% and 90% between 40% and 80%, between 40% and 70%, between 40% and 60%, between 40% and 50%, between 50% and 90%, between 50% and 80%, between 50% and 70%, between 50% and 60%, between 60% and 90%, between 60% and 80%, between 60% and 70%, between 70% and 90%, or between 70% and 80% less or lower than in the same tissue(s) of a wild-type or control corn plant. A modified corn plant having a reduced or eliminated expression level of a br2 gene in one or more tissue(s), may also be substantially free of off-types, such as male reproductive tissues or structures and/or other off-types in at least one ear of the modified corn plant.

Methods and techniques are provided for screening for, and/or identifying, cells or plants, etc., for the presence of targeted edits or transgenes, and selecting cells or plants comprising targeted edits or transgenes, which may be based on one or more phenotypes or traits, or on the presence or absence of a molecular marker or polynucleotide or protein sequence in the cells or plants. Nucleic acids can be isolated and detected using techniques known in the art. For example, nucleic acids can be isolated and detected using, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis. Any method known in the art may be used to screen for, and/or identify, cells, plants, etc., having a transgene or genome edit in its genome, which may be based on any suitable form of visual observation, selection, molecular technique, etc.

In some embodiments, methods are provided for detecting recombinant nucleic acids and/or polypeptides in plant cells. For example, nucleic acids may be detected using hybridization probes or through production of amplicons using PCR with primers as known in the art. Hybridization between nucleic acids is discussed in Sambrook et al. (1989, Molecular Cloning: A Laboratory Manual, 2nd Ed., Cold Spring Harbor Laboratory Press, Cold Spring Harbor, NY). Polypeptides can be detected using antibodies. Techniques for detecting polypeptides using antibodies include enzyme linked immunosorbent assays (ELISAs), Western blots, immunoprecipitations, immunofluorescence, and the like. An antibody provided herein may be a polyclonal antibody or a monoclonal antibody. An antibody having specific binding affinity for a polypeptide provided herein can be generated using methods known in the art. An antibody or hybridization probe may be attached to a solid support, such as a tube, plate or well, using methods known in the art.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials.

The screening and selection of modified, edited or transgenic plants or plant cells can be through any methodologies known to those skilled in the art of molecular biology. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

According to embodiments of the present disclosure, a modified or transgenic corn plant or plant part, one or more modified or transgenic corn plants or plant parts, or a plurality modified or transgenic corn plants or plant parts as provided herein, or an agricultural field or soil in which a modified or transgenic corn plant or plant part, one or more modified or transgenic corn plants or plant parts or a plurality modified or transgenic corn plants or plant parts as provided herein are planted or grown, can be treated with an agricultural composition comprising one or more active ingredients or other agents, such as, for example and without limitation, a pesticide or one or more pesticides, an herbicide or one or more herbicides, a fungicide or one or more fungicides, an insecticide or one or more insecticides, a plant growth regulator or plant stimulant or one or more plant growth regulators and/or plant stimulants, and/or a safener or one or more safeners. Provided below are lists of possible or representative compounds for each of these types of actives or agents, and an agricultural composition may comprise one or any combination or multiplicity of these actives, agents or compounds. Such an agricultural composition may be applied, for example, as a foliar, soil or in-furrow treatment, as a pre-emergent, pre-sowing and/or post-emergent treatment, and/or in some cases, may be applied to modified or transgenic plant part or seed provided herein.

A modified or transgenic corn plant or plant part, one or more modified or transgenic corn plants or plant parts, or plurality modified or transgenic corn plants or plant parts, which may be further planted or grown in a greenhouse or an agricultural field or soil, that is/are treated with an agricultural composition as provided herein, may comprise any mutation, edit or other genetic modification of a brachytic locus or gene, such as a brachytic2 (br2) locus or gene, including any mutant, edited or modified allele (collectively any “mutant allele”) of the brachytic or brachytic2 (br2) gene or locus, and may be homozygous, bi-allelic or heterozygous for one or more mutant allele(s) of the brachytic or brachytic2 (br2) gene or locus. See, e.g., PCT Application No. PCT/US2016/029492 and PCT/US2017/067888, and Bage, S. A. et al., “Genetic characterization of novel and CRISPR-Cas9 gene edited maize brachytic 2 alleles,” Plant Gene, 21:100198 (2020), the entire contents and disclosures of which are incorporated herein by reference. The mutant allele of an endogenous brachytic or brachytic2 (br2) gene or locus may be any loss-of-function mutation of the brachytic or brachytic2 (br2) gene or locus, which may comprise a deletion(s), inversion(s), insertion(s), and/or substitution(s) of one or more nucleotides of the brachytic or brachytic2 (br2) gene or locus, or a combination thereof. The mutant allele of the endogenous brachytic or brachytic2 (br2) gene or locus may comprise any dominant, semi-dominant or recessive mutant allele or mutation, edit or other genetic modification of the brachytic or brachytic2 (br2) gene or locus. The mutant allele may comprise a missense, nonsense and/or frameshift mutation(s) and/or a premature stop codon. The mutant allele of the brachytic2 (br2) gene or locus in corn may be any of the North American, Mexican or Italian alleles, or from any of the North American, Mexican or Italian lines, identified in PCT Application No. PCT/US2016/029492 or PCT/US2017/067888, and/or Bage, S. A. et al., “Genetic characterization of novel and CRISPR-Cas9 gene edited maize brachytic 2 alleles,” Plant Gene, 21:100198 (2020). The mutant allele of the brachytic2 (br2) gene or locus in corn may be a br2-7081 or br2-7861 allele. The mutant allele of the brachytic2 (br2) gene or locus in corn may be a br2-1005 allele. Alternatively, the mutant allele of an endogenous brachytic or brachytic2 (br2) gene or locus may comprise a DNA segment inserted into the endogenous brachytic or br2 gene or locus, wherein the DNA segment encodes an antisense RNA sequence that is complementary to consecutive nucleotides of the endogenous brachytic or br2 gene or locus as provided herein. The mutant allele may comprise one or more natural or non-natural mutation(s), edit(s) and/or other genetic modification(s) of a brachytic locus or gene, such as a brachytic2 (br2) locus or gene. The natural or native mutation refers to a mutation that occurs spontaneously in nature without any involvement of laboratory or experimental procedures or under the exposure to mutagens. The non-natural mutation, edit or other genetic modification refers to a mutation that is not spontaneously occurring in nature but as a result of a laboratory or experimental procedure, such as a genome editing technique or exposure to a mutagen. The mutant may not comprise a br2-23 or SNP5259 brachytic mutation. See, e.g., Pilu et al., Molecular Breeding, 20: 83-91(2007), Xing et al., J. Exp. Bot., 66: 3791-802 (2015), and Cassani et al., Plant Growth Regul., 64: 185-192 (2011).

An agricultural composition may be formulated according to its intended use and application. The appropriate formulation of the agricultural composition may be chosen to have different physicochemical parameters, components and stabilities of the respective compound(s). Possible types of formulations for an agricultural composition can include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes. If appropriate, some agricultural compositions of a pesticidal compound or one or more pesticidal compounds might be formulated and used as a seed coating applied to a plant part or seed as provided herein.

Classes of herbicides that might be used in an agricultural composition for controlling agriculturally harmful plants (i.e., weeds) can be based on inhibition of, for example but without limitation, acetolactate synthase, acetyl-CoA carboxylase, cellulose synthase, enolpyruvylshikimate-3-phosphate synthase, glutamine synthetase, p-hydroxyphenylpyruvate dioxygenase, phytoendesaturase, photosystem I, photosystem II, protoporphyrinogen oxidase, as described, for example, in Weed Research 26 (1986) 441-445 or “The Pesticide Manual”, 16th edition, The British Crop Protection Council and the Royal Soc. of Chemistry, 2012 and literature cited therein. An agricultural composition comprising one or more herbicides can be applied, for example, by pre-sowing (if appropriate also by incorporation into the soil), pre-emergence and/or post-emergence processes.

Specific herbicides that might be used in an agricultural composition of the present disclosure may include, for example: acetochlor, acifluorfen, acifluorfen-methyl, acifluorfen-sodium, aclonifen, alachlor, allidochlor, alloxydim, alloxydim-sodium, ametryn, amicarbazone, amidochlor, amidosulfuron,4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, aminopyralid-dimethylammonium, aminopyralid-tripromine, amitrole, ammoniumsulfamate, anilofos, asulam, asulam-potassium, asulam sodium, atrazine, azafenidin, azimsulfuron, beflubutamid, (S)-(−)-beflubutamid, beflubutamid-M, benazolin, benazolin-ethyl, benazolin-dimethylammonium, benazolin-potassium, benfluralin, benfuresate, bensulfuron, bensulfuron-methyl, bensulide, bentazone, bentazone-sodium, benzobicyclon, benzofenap, bicyclopyrone, bifenox, bilanafos, bilanafos-sodium, bipyrazone, bispyribac, bispyribac-sodium, bixlozone (F-9600), bromacil, bromacil-lithium, bromacil-sodium, bromobutide, bromofenoxim, bromoxynil, bromoxynil-butyrate, -potassium, -heptanoate und -octanoate, busoxinone, butachlor, butafenacil, butamifos, butenachlor, butralin, butroxydim, butylate, cafenstrole, cambendichlor, carbetamide, carfentrazone, carfentrazone-ethyl, chloramben, chloramben-ammonium, chloramben-diolamine, chlroamben-methyl, chloramben-methylammonium, chloramben-sodium, chlorbromuron, chlorfenac, chlorfenac-ammonium, chlorfenac-sodium, chlorfenprop, chlorfenprop-methyl, chlorflurenol, chlorflurenol-methyl, chloridazon, chlorimuron, chlorimuron-ethyl, chlorophthalim, chlorotoluron, chlorsulfuron, chlorthal, chlorthal-dimethyl, chlorthal-monomethyl, cinidon, cinidon-ethyl, cinmethylin, exo-(+)-cinmethylin or exo-(−)-cinmethylin, i.e. (1R,2S,4S)-4-isopropyl-1-methyl-2-[(2-methylbenzyl)oxy]-7-oxabicyclo[2.2.1]heptane, cinosulfuron, clacyfos, clethodim, clodinafop, clodinafop-ethyl, clodinafop-propargyl, clomazone, clomeprop, clopyralid, clopyralid-methyl, clopyralid-olamine, clopyralid-potassium, clopyralid-tripomine, cloransulam, cloransulam-methyl, cumyluron, cyanamide, cyanazine, cycloate, cyclopyranil, cyclopyrimorate, cyclosulfamuron, cycloxydim, cyhalofop, cyhalofop-butyl, cyprazine, 2,4-D (including theammonium, butotyl, -butyl, choline, diethylammonium, -dimethylammonium, -diolamine, -doboxyl, -dodecylammonium, etexyl, ethyl, 2-ethylhexyl, heptylammonium, isobutyl, isooctyl, isopropyl, isopropylammonium, lithium, meptyl, methyl, potassium, tetradecylammonium, triethylammonium, triisopropanolammonium, tripromine and trolamine salt thereof), 2,4-DB, 2,4-DB-butyl, -dimethylammonium, isooctyl, -potassium und -sodium, daimuron (dymron), dalapon, dalapon-calcium, dalapon-magnesium, dalapon-sodium, dazomet, dazomet-sodium, n-decanol, 7-deoxy-D-sedoheptulose, desmedipham, detosyl-pyrazolate (DTP), dicamba and its salts, e. g. dicamba-biproamine, dicamba-N,N-Bis(3-aminopropyl)methylamine, dicamba-butotyl, dicamba-choline, dicamba-diglycolamine, dicamba-dimethylammonium, dicamba-diethanolaminemmonium, dicamba-diethylammonium, dicamba-isopropylammonium, dicamba-methyl, dicamba-monoethanolaminedicamba-olamine, dicamba-potassium, dicamba-sodium, dicamba-triethanolamine, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-butotyl, dichlroprop-dimethylammonium, dichhlorprop-etexyl, dichlorprop-ethylammonium, dichlorprop-isoctyl, dichlorprop-methyl, dichlorprop-postassium, dichlorprop-sodium, dichlorprop-P, dichlorprop-P-dimethylammonium, dichlorprop-P-etexyl, dichlorprop-P-potassium, dichlorprop-sodium, diclofop, diclofop-methyl, diclofop-P, diclofop-P-methyl, diclosulam, difenzoquat, difenzoquat-metilsulfate, diflufenican, diflufenzopyr, diflufenzopyr-sodium, dimefuron, dimepiperate, dimesulfazet, dimethachlor, dimethametryn, dimethenamid, dimethenamid-P, dimetrasulfuron, dinitramine, dinoterb, dinoterb-acetate, diphenamid, diquat, diquat-dibromid, diquat-dichloride, dithiopyr, diuron, DNOC, DNOC-ammonium, DNOC-potassium, DNOC-sodium, endothal, endothal-diammonium, endothal-dipotassium, endothal-disodium, Epyrifenacil (S-3100), EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-5231, i.e. N-[2-Chlor-4-fluor-5-[4-(3-fluorpropyl)-4,5-dihydro-5-oxo-1H-tetrazol-1-yl]-phenyl]-ethansulfonamid, F-7967, i.e. 3-[7-Chlor-5-fluor-2-(trifluormethyl)-1H-benzimidazol-4-yl]-1-methyl-6-(trifluormethyl)pyrimidin-2,4(1H,3H)-dion, fenoxaprop, fenoxaprop-P, fenoxaprop-ethyl, fenoxaprop-P-ethyl, fenoxasulfone, fenpyrazone, fenquinotrione, fentrazamide, flamprop, flamprop-isoproyl, flamprop-methyl, flamprop-M-isopropyl, flamprop-M-methyl, flazasulfuron, florasulam, florpyrauxifen, florpyrauxifen-benzyl, fluazifop, fluazifop-butyl, fluazifop-methyl, fluazifop-P, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium und -methyl, fluoroglycofen, fluoroglycofen-ethyl, flupropanate, flupropanate-sdium, flupyrsulfuron, flupyrsulfuron-methyl, flupyrsulfuron-methyl-sodium, fluridone, flurochloridone, fluroxypyr, fluroxypyr-butometyl, fluroxypyr-meptyl, flurtamone, fluthiacet, fluthiacet-methyl, fomesafen, fomesafen-sodium, foramsulfuron, foramsulfuron sodium salt, fosamine, fosamine-ammonium, glufosinate, glufosinate-ammonium, glufosinate-sodium, L-glufosinate-ammonium, L-glufosiante-sodium, glufosinate-P-sodium, glufosinate-P-ammonium, glyphosate, glyphosate-ammonium, -isopropyl-ammonium, -diammonium, -dimethylammonium, -potassium, -sodium, sesquisodium and -trimesium, H-9201, i.e. O-(2,4-Dimethyl-6-nitrophenyl)-O-ethyl-isopropylphosphoramidothioat, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, haloxifop-sodium, hexazinone, HNPC-A8169, i.e. prop-2-yn-1-yl (2S)-2-{3-[(5-tert-butylpyridin-2-yl)oxy]phenoxy}propanoate, HW-02, i.e. 1-(Dimethoxyphosphoryl)-ethyl-(2,4-dichlorphenoxy)acetat, hydantocidin, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazaquin.methyl, imazethapyr, imazethapyr-immonium, imazosulfuron, indanofan, indaziflam, iodosulfuron, iodosulfuron-methyl, iodosulfuron-methyl-sodium, ioxynil, ioxynil-lithium, -octanoate, -potassium und sodium, ipfencarbazone, isoproturon, isouron, isoxaben, isoxaflutole, karbutilate, KUH-043, i.e. 3-({[5-(Difluormethyl)-1-methyl-3-(trifluormethyl)-1H-pyrazol-4-yl]methyl}sulfonyl)-5,5-dimethyl-4,5-dihydro-1,2-oxazol,ketospiradox, ketospiradox-potassium, lactofen, lenacil, linuron, MCPA, MCPA-butotyl, -butyl, -dimethylammonium, -diolamine, -2-ethylhexyl, -ethyl, -isobutyl, isoctyl, -isopropyl, -isopropylammonium, -methyl, olamine, -potassium, -sodium and -trolamine, MCPB, MCPB-methyl, -ethyl und -sodium, mecoprop, mecoprop-butotyl, mecoprop-demethylammonium, mecoprop-diolamine, mecoprop-etexyl, mecoprop-ethadyl, mecoprop-isoctyl, mecoprop-methyl, mecoprop-potassium, mecoprop-sodium, and mecoprop-trolamine, mecoprop-P, mecoprop-P-butotyl, -dimethylammonium, -2-ethylhexyl and -potassium, mefenacet, mefluidide, mefluidide-diolamine, mefluidide-potassium, mesosulfuron, mesosulfuron-methyl, mesosulfuron sodium salt, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinate, monolinuron, monosulfuron, monosulfuron-methyl, MT-5950, i.e. N-[3-chlor-4-(1-methylethyl)-phenyl]-2-methylpentanamid, NGGC-011, napropamide, NC-310, i.e. 4-(2,4-Dichlorbenzoyl)-1-methyl-5-benzyloxypyrazol, NC-656, i.e. 3-[(isopropylsulfonyl)methyl]-N-(5-methyl-1,3,4-oxadiazol-2-yl)-5-(trifluoromethyl)[1,2,4]triazolo[4,3-a]pyridine-8-carboxamide, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefone, oxyfluorfen, paraquat, paraquat-dichloride, paraquat-dimethylsulfate, pebulate, pendimethalin, penoxsulam, pentachlorphenol, pentoxazone, pethoxamid, petroleum oils, phenmedipham, phenmedipham-ethyl, picloram, picloram-dimethylammonium, picloram-etexyl, picloram-isoctyl, picloram-methyl, picloram-olamine, picloram-potassium, picloram-triethylammonium, picloram-tripromine, picloram-trolamine, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinclorac-dimethylammonium, quinclorac-methyl, quinmerac, quinoclamine, quizalofop, quizalofop-ethyl, quizalofop-P, quizalofop-P-ethyl, quizalofop-P-tefuryl, QYM201, i.e. 1-{2-chloro-3-[(3-cyclopropyl-5-hydroxy-1-methyl-1H-pyrazol-4-yl)carbonyl]-6-(trifluoromethyl)phenyl}piperidin-2-one, rimsulfuron, saflufenacil, sethoxydim, siduron, simazine, simetryn, SL-261, sulcotrione, sulfentrazone, sulfometuron, sulfometuron-methyl, sulfosulfuron, SYP-249, i.e. 1-Ethoxy-3-methyl-1-oxobut-3-en-2-yl-5-[2-chlor-4-(trifluormethyl)phenoxy]-2-nitrobenzoat, SYP-300, i.e. 1-[7-Fluor-3-oxo-4-(prop-2-in-1-yl)-3,4-dihydro-2H-1,4-benzoxazin-6-yl]-3-propyl-2-thioxoimidazolidin-4,5-dion, 2,3,6-TBA, TCA (trichloro acetic acid) and its salts, e.g. TCA-ammonium, TCA-calcium, TCA-ethyl, TCA-magnesium, TCA-sodium, tebuthiuron, tefuryltrione, tembotrione, tepraloxydim, terbacil, terbucarb, terbumeton, terbuthylazine, terbutryn, tetflupyrolimet, thaxtomin, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, triclopyr-butotyl, triclopyr-choline, triclopyr-ethyl, triclopyr-triethylammonium, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-Dichlor-N-{2-[(4,6-dimethoxypyrimidin-2-yl)oxy]benzyl}anilin, 3-(2-chloro-4-fluoro-5-(3-methyl-2,6-dioxo-4-trifluoromethyl-3,6-dihydropyrimidin-1 (2H)-yl)phenyl)-5-methyl-4,5-dihydroisoxazole-5-carboxylic acid ethyl ester, 3-chloro-2-[3-(difluoromethyl)isoxazolyl-5-yl]phenyl-5-chloropyrimidin-2-yl ether, 2-(3,4-dimethoxyphenyl)-4-[(2-hydroxy-6-oxocyclohex-1-en-1-yl)carbonyl]-6-methylpyridazine-3(2H)-one, 2-((2-[(2-methoxyethoxy)methyl]-6-methylpyridin-3-yl)carbonyl)cyclohexane-1,3-dione, (5-hydroxy-1-methyl-1H-pyrazol-4-yl)(3,3,4-trimethyl-1,1-dioxido-2,3-dihydro-1-benzothiophen-5-yl)methanone, 1-methyl-4-[(3,3,4-trimethyl-1,1-dioxido-2,3-dihydro-1-benzothiophen-5-yl)carbonyl]-1H-pyrazol-5-yl propane-1-sulfonate, 4-{2-chloro-3-[(3,5-dimethyl-1H-pyrazol-1-yl)methyl]-4-(methylsulfonyl)benzoyl}-1-methyl-1H-pyrazol-5-yl 1,3-dimethyl-1H-pyrazole-4-carboxylate; cyanomethyl 4-amino-3-chloro-5-fluoro-6-(7-fluoro-1H-indol-6-yl)pyridine-2-carboxylate, prop-2-yn-1-yl 4-amino-3-chloro-5-fluoro-6-(7-fluoro-1H-indol-6-yl)pyridine-2-carboxylate, methyl 4-amino-3-chloro-5-fluoro-6-(7-fluoro-1H-indol-6-yl)pyridine-2-carboxylate, 4-amino-3-chloro-5-fluoro-6-(7-fluoro-1H-indol-6-yl)pyridine-2-carboxylic acid, benzyl 4-amino-3-chloro-5-fluoro-6-(7-fluoro-1H-indol-6-yl)pyridine-2-carboxylate, ethyl 4-amino-3-chloro-5-fluoro-6-(7-fluoro-1H-indol-6-yl)pyridine-2-carboxylate, methyl 4-amino-3-chloro-5-fluoro-6-(7-fluoro-1-isobutyryl-1H-indol-6-yl)pyridine-2-carboxylate, methyl 6-(1-acetyl-7-fluoro-1H-indol-6-yl)-4-amino-3-chloro-5-fluoropyridine-2-carboxylate, methyl 4-amino-3-chloro-6-[1-(2,2-dimethylpropanoyl)-7-fluoro-1H-indol-6-yl]-5-fluoropyridine-2-carboxylate, methyl 4-amino-3-chloro-5-fluoro-6-[7-fluoro-1-(methoxyacetyl)-1H-indol-6-yl]pyridine-2-carboxylate, potassium 4-amino-3-chloro-5-fluoro-6-(7-fluoro-1H-indol-6-yl)pyridine-2-carboxylate, sodium 4-amino-3-chloro-5-fluoro-6-(7-fluoro-1H-indol-6-yl)pyridine-2-carboxylate, butyl 4-amino-3-chloro-5-fluoro-6-(7-fluoro-1H-indol-6-yl)pyridine-2-carboxylate, 4-hydroxy-1-methyl-3-[4-(trifluoromethyl)pyridin-2-yl]imidazolidin-2-one, 3-(5-tert-butyl-1,2-oxazol-3-yl)-4-hydroxy-1-methylimidazolidin-2-one.

Specific plant growth regulators or plant stimulants that may be used in an agricultural composition of the present disclosure include, for example: abscisic acid, acibenzolar, acibenzolar-S-methyl, 1-aminocyclopro-1-yl carboxylic acid and derivatives thereof, 5-Aminolavulinsaure, ancymidol, 6-benzylaminopurine, brassinolide, brassinolide-ethyl, catechin, chitinous compounds, chitooligosaccharides (COs), lipochitooligosaccharides (LCOs), chlormequat chloride, cloprop, cyclanilide, 3-(Cycloprop-1-enyl)propionic acid, daminozide, dazomet, dazomet-sodium, n-decanol, dikegulac, dikegulac-sodium, endothal, endothal-dipotassium, -disodium, and mono(N,N-dimethylalkylammonium), ethephon, flumetralin, flurenol, flurenol-butyl, flurenol-methyl, flurprimidol, forchlorfenuron, gibberellic acid, inabenfide, indol-3-acetic acid (IAA), 4-indol-3-ylbutyric acid, isoprothiolane, probenazole, jasmonic acid, Jasmonic acid or derivatives thereof (e.g. Jasmonic acid methyl ester), lipo-chitooligosaccharides, linoleic acid or derivatives thereof, linolenic acid or derivatives thereof, maleic hydrazide, mepiquat chloride, mepiquat pentaborate, 1-methylcyclopropene, 3′-methyl abscisic acid, 2-(1-naphthyl)acetamide, 1-naphthylacetic acid, 2-naphthyloxyacetic acid, nitrophenolate-mixture, 4-Oxo-4[(2-phenylethyl)amino]butyric acid, paclobutrazol, 4-phenylbutyric acid, N-phenylphthalamic acid, prohexadione, prohexadione-calcium, prohydrojasmon, salicylic acid, salicylic acid methyl ester, strigolacton, tecnazene, thidiazuron, triacontanol, trinexapac, trinexapac-ethyl, tsitodef, uniconazole, uniconazole-P, 2-fluoro-N-(3-methoxyphenyl)-9H-purin-6-amine. COs, sometimes referred to as N-acetyl chitooligosaccharides, are also composed of GlcNAc residues but have side chain decorations that make them different from chitin molecules [(C₈H₁₃NO₅)ₙ, CAS No. 1398-61-4] and chitosan molecules [(C₅H₁₁NO₄)ₙ, CAS No. 9012-76-4]). LCOs are similar to COs but with a pendant fatty acid chain. As understood in the art, LCOs differ in the number of GlcNAc residues in the backbone, in the length and degree of saturation of the fatty acyl chain and in the substitutions of reducing and non-reducing sugar residues). LCO, sometimes referred to as symbiotic nodulation (Nod) signals (or Nod factors) or as Myc factors, consist of an oligosaccharide backbone of β-1,4-linked N-acetyl-D-glucosamine (“GlcNAc”) residues with an N-linked fatty acyl chain condensed at the non-reducing end.

Specific safeners (reducing the phytotoxic side effects of the herbicides/pesticides employed by being applied in an effective amount) that may be used in an agricultural composition of the present disclosure include, for example:
S1) Compounds from the group of heterocyclic carboxylic acid derivatives:
   S1^{a}) Compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described, e.g., in WO-A-91/07874;
   S1^{b}) Derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described, e.g., in EP-A-333131 and EP-A-269806;
   S1^{c}) Derivatives of 1,5-diphenylpyrazole-3-carboxylic acid (Si:), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described, e.g., in EP-A-268554;
   S1^{d}) Compounds of the triazolecarboxylic acid type (S1^{d}), preferably compounds such as fenchlorazole(-ethyl ester), i.e. ethyl 1-(2,4-dichlorophenyl)-5-trichloromethyl-(1H)-1,2,4-triazole-3-carboxylate (S1-7), and related compounds, as described, e.g., in EP-A-174562 and EP-A-346620;
   S1^{e}) Compounds of the 5-benzyl- or 5-phenyl-2-isoxazoline-3-carboxylic acid or of the 5,5-diphenyl-2-isoxazoline-3-carboxylic acid type (Sic), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazolinecarboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13) as described, e.g., in patent application WO-A-95/07897.
S2) Compounds from the group of the 8-quinolinoxy derivatives (S2):
   S2^{a}) Compounds of the 8-quinolinoxyacetic acid type (S2^{a}), preferably 1-methylhexyl (5-chloro-8-quinolinoxy)acetate (“cloquintocet-mexyl”) (S2-1), 1,3-dimethylbut-1-yl (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5), methyl (5-chloro-8-quinolinoxy)acetate (S2-6), allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86750, EP-A-94349 and EP-A-191736 or EP-A-0 492 366, and also (5-chloro-8-quinolinoxy)acetic acid (S2-10), hydrates and salts thereof, for example the lithium, sodium, potassium, calcium, magnesium, aluminum, iron, ammonium, quaternary ammonium, sulfonium or phosphonium salts thereof, as described, e.g., in WO-A-2002/34048;
   S2^{b}) Compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described, e.g., in EP-A-0 582 198.
S3) Active compounds of the dichloroacetamide type (S3), which are frequently used as pre-emergence safeners (soil-acting safeners), for example “dichlormid” (N,N-diallyl-2,2-dichloroacetamide) (S3-1), “R-29148” (3-dichloroacetyl-2,2,5-trimethyl-1,3-oxazolidine) from Stauffer (S3-2), “R-28725” (3-dichloroacetyl-2,2-dimethyl-1,3-oxazolidine) from Stauffer (S3-3), “benoxacor” (4-dichloroacetyl-3,4-dihydro-3-methyl-2H-1,4-benzoxazine) (S3-4), “PPG-1292” (N-allyl-N-[(1,3-dioxolan-2-yl)methyl]dichloroacetamide) from PPG Industries (S3-5), “DKA-24” (N-allyl-N-[(allylaminocarbonyl)methyl]dichloroacetamide) from Sagro-Chem (S3-6), “AD-67” or “MON 4660” (3-dichloroacetyl-1-oxa-3-azaspiro[4.5]decane) from Nitrokemia or Monsanto (S3-7), “TI-35” (1-dichloroacetylazepane) from TRI-Chemical RT (S3-8), “diclonon” (dicyclonon) or “BAS145138” or “LAB145138” (S3-9) ((RS)-1-dichloroacetyl-3,3,8a-trimethylperhydropyrrolo[1,2-a]pyrimidin-6-one) from BASF, “furilazole” or “MON 13900” ((RS)-3-dichloroacetyl-5-(2-furyl)-2,2-dimethyloxazolidine) (S3-10), and the (R) isomer thereof (S3-11).
S4) Compounds from the class of the acylsulfonamides (S4):
   S4^{a})N-Acylsulfonamides of the formula (S4^{a}) and salts thereof, as described, e.g., in WO-A-97/45016,

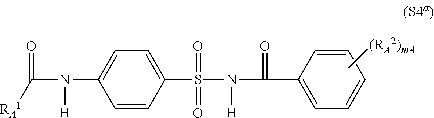

in which R_{A}¹ represents (C₁-C₆)-alkyl, (C₃-C₆)-cycloalkyl, where the 2 latter radicals are substituted by v_{A} substituents from the group of halogen, (C₁-C₄)-alkoxy, (C₁-C₆)-haloalkoxy and (C₁-C₄)-alkylthio and, in the case of cyclic radicals, also by (C₁-C₄)-alkyl and (C₁-C₄)-haloalkyl; R_{A}² represents halogen, (C₁-C₄)-alkyl, (C₁-C₄)-alkoxy, CF₃; m_{A} represents 1 or 2; v_{A} represents 0, 1, 2 or 3;
S4^{b}) Compounds of the 4-(benzoylsulfamoyl)benzamide type of the formula (S4^{b}) and salts thereof, as described, e.g., in WO-A-99/16744,

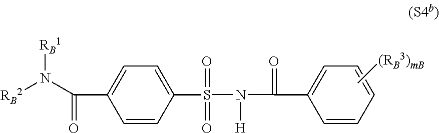

in which R_{B}¹, R_{B}² independently of one another represent hydrogen, (C₁-C₆)-alkyl, (C₃-C₆)-cycloalkyl, (C₃-C₆)-alkenyl, (C₃-C₆)-alkynyl, R_{B}³ represents halogen, (C₁-C₄)-alkyl, (C₁-C₄)-haloalkyl or (C₁-C₄)-alkoxy and m_{B} represents 1 or 2, e.g. those in which R_{B}¹=cyclopropyl, R_{B}²=hydrogen and (R_{B}³)=2-OMe (“cyprosulfamide”, S4-1), R_{B}¹=cyclopropyl, R_{B}²=hydrogen and (R_{B}³)=5-Cl-2-OMe (S4-2), R_{B}¹=ethyl, R_{B}²=hydrogen and (R_{B}³)=2-OMe (S4-3), R_{B}¹=isopropyl, R_{B}²=hydrogen and (R_{B}³)=5-Cl-2-OMe (S4-4) and R_{B}¹=isopropyl, R_{B}²=hydrogen and (R_{B}³)=2-OMe (S4-5);
S4^{c}) Compounds from the class of the benzoylsulfamoylphenylureas of the formula (S4), as described, e.g., in EP-A-365484,

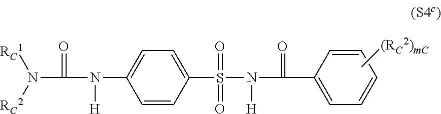

in which R_{C}¹, R_{C}² independently of one another represent hydrogen, (C₁-C₈)-alkyl, (C₃-C₈)-cycloalkyl, (C₃-C₆)-alkenyl, (C₃-C₆)-alkynyl, R_{C}³ represents halogen, (C₁-C₄)-alkyl, (C₁-C₄)-alkoxy, CF₃ and m_{C} represents 1 or 2; for example 1-[4-(N-2-methoxybenzoylsulfamoyl)phenyl]-3-methylurea, 1-[4-(N-2-methoxybenzoylsulfamoyl)phenyl]-3,3-dimethylurea, 1-[4-(N-4,5-dimethylbenzoylsulfamoyl)phenyl]-3-methylurea;
S₄^{d}) Compounds of the N-phenylsulfonylterephthalamide type of the formula (S4^{d}) and salts thereof, which are described, e.g., in CN 101838227,

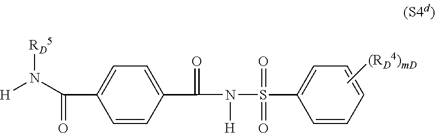

in which R_{D}⁴ represents halogen, (C₁-C₄)-alkyl, (C₁-C₄)-alkoxy, CF₃; m_{D} represents 1 or 2; R_{D}⁵ represents hydrogen, (C₁-C₆)-alkyl, (C₃-C₆)-cycloalkyl, (C₂-C₆)-alkenyl, (C₂-C₆)-alkynyl, (C₅-C₆)-cycloalkenyl.
S5) Active compounds in the class of hydroxyaromatics and the aromatic-aliphatic carboxylic acid derivatives (S5), for example ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.
S6) Active compounds in the class of 1,2-dihydroquinoxalin-2-ones (S6), for example 1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described, e.g., in WO-A-2005/112630.
S7) Compounds in the class of diphenylmethoxyacetic acid derivatives (S7), e.g. methyl diphenylmethoxyacetate (CAS Reg. No. 41858-19-9) (S7-1), ethyl diphenylmethoxyacetate or diphenylmethoxyacetic acid, as described, e.g., in WO-A-98/38856.
S8) Compounds of the formula (S8), as described, e.g., in WO-A-98/27049,

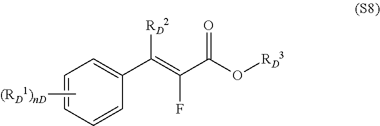

in which the symbols and indices are defined as follows: R_{D}¹ represents halogen, (C₁-C₄)-alkyl, (C₁-C₄)-haloalkyl, (C₁-C₄)-alkoxy, (C₁-C₄)-haloalkoxy, R_{D}² represents hydrogen or (C₁-C₄)-alkyl, R_{D}³ represents hydrogen, (C₁-C₈)-alkyl, (C₂-C₄)-alkenyl, (C₂-C₄)-alkynyl or aryl, where each of the aforementioned carbon-containing radicals is unsubstituted or substituted by one or more, preferably up to three, identical or different radicals from the group consisting of halogen and alkoxy; or salts thereof, n_{D} represents an integer from 0 to 2.
S9) Active compounds from the class of the 3-(5-tetrazolylcarbonyl)-2-quinolones (S9), for example 1,2-dihydro-4-hydroxy-1-ethyl-3-(5-tetrazolylcarbonyl)-2-quinolone (CAS Reg. No.: 219479-18-2), 1,2-dihydro-4-hydroxy-1-methyl-3-(5-tetrazolylcarbonyl)-2-quinolone (CAS Reg. No. 95855-00-8), as described, e.g., in WO-A-1999/000020.
S10) Compounds of the formula (S10^{a}) or (S10^{b}) as described, e.g., in WO-A-2007/023719 and WO-A-2007/023764

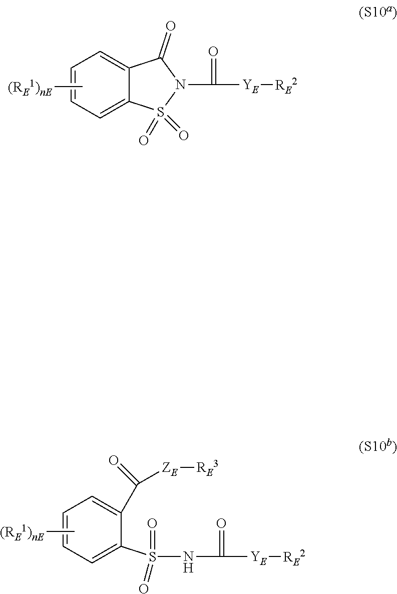

in which R_{E}¹ represents halogen, (C₁-C₄)-alkyl, methoxy, nitro, cyano, CF₃, OCF₃, Y_{E}, Z_{E} independently of one another represent O or S, n_{E} represents an integer from 0 to 4, R_{E}² represents (C₁-C₁₆)-alkyl, (C₂-C₆)-alkenyl, (C₃-C₆)-cycloalkyl, aryl; benzyl, halobenzyl, R_{E}³ represents hydrogen or (C₁-C₆)-alkyl.
S11) Active compounds of the oxyimino compounds type (S11), which are known as seed-dressing agents, for example “oxabetrinil” ((Z)-1,3-dioxolan-2-ylmethoxyimino(phenyl)acetonitrile) (S11-1), which is known as a seed-dressing safener for millet/sorghum against metolachlor damage, “fluxofenim” (1-(4-chlorophenyl)-2,2,2-trifluoro-1-ethanone O-(1,3-dioxolan-2-ylmethyl)oxime) (S11-2), which is known as a seed-dressing safener for millet/sorghum against metolachlor damage, and “cyometrinil” or “CGA-43089” ((Z)-cyanomethoxyimino(phenyl)acetonitrile) (S11-3), which is known as a seed-dressing safener for millet/sorghum against metolachlor damage.
S12) Active compounds from the class of the isothiochromanones (S12), for example methyl [(3-oxo-1H-2-benzothiopyran-4(3H)-ylidene)methoxy]acetate (CAS Reg. No. 205121-04-6) (S12-1) and related compounds from WO-A-1998/13361.
S13) One or more compounds from group (S13): “naphthalic anhydride” (1,8-naphthalenedicarboxylic anhydride) (S13-1), which is known as a seed-dressing safener for corn against thiocarbamate herbicide damage, “fenclorim” (4,6-dichloro-2-phenylpyrimidine) (S13-2), which is known as a safener for pretilachlor in sown rice, “flurazole” (benzyl 2-chloro-4-trifluoromethyl-1,3-thiazole-5-carboxylate) (S13-3), which is known as a seed-dressing safener for millet/sorghum against alachlor and metolachlor damage, “CL 304415” (CAS Reg. No. 31541-57-8) (4-carboxy-3,4-dihydro-2H-1-benzopyran-4-acetic acid) (S13-4) from American Cyanamid, which is known as a safener for corn against damage by imidazolinones, “MG 191” (CAS Reg. No. 96420-72-3) (2-dichloromethyl-2-methyl-1,3-dioxolane) (S13-5) from Nitrokemia, which is known as a safener for corn, “MG 838” (CAS Reg. No. 133993-74-5) (2-propenyl 1-oxa-4-azaspiro[4.5]decane-4-carbodithioate) (S13-6) from Nitrokemia, “disulfoton” (0,0-diethyl S-2-ethylthioethyl phosphorodithioate) (S13-7), “dietholate” (0,0-diethyl 0-phenyl phosphorothioate) (S13-8), “mephenate” (4-chlorophenyl methylcarbamate) (S13-9).
S14) Active compounds, for example “dimepiperate” or “MY-93” (S-1-methyl 1-phenylethylpiperidine-1-carbothioate), which is known as a safener for rice against damage by the herbicide molinate, “daimuron” or “SK 23” (1-(1-methyl-1-phenylethyl)-3-p-tolylurea), which is known as a safener for rice against damage by the herbicide imazosulfuron, “cumyluron”=“JC-940” (3-(2-chlorophenylmethyl)-1-(1-methyl-1-phenylethyl)urea, see JP-A-60087270), which is known as a safener for rice against damage by some herbicides, “methoxyphenone” or “NK 049” (3,3′-dimethyl-4-methoxybenzophenone), which is known as a safener for rice against damage by some herbicides, “CSB” (1-bromo-4-(chloromethylsulfonyl)benzene) from Kumiai, (CAS Reg. No. 54091-06-4), which is known as a safener against damage by some herbicides in rice.
S15) Compounds of the formula (S15) or tautomers thereof.

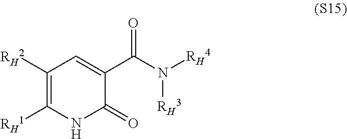

as described, e.g., in WO-A-2008/131861 and WO-A-2008/131860
in which R_{H}¹ represents a (C₁-C₆)-haloalkyl radical and R_{H}² represents hydrogen or halogen and R_{H}³, R_{H}⁴ independently of one another represent hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted, or (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring, where each of the 4 latter radicals is unsubstituted or substituted by one or more radicals from the group of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted, or R_{H}³ represents (C₁-C₄)-alkoxy, (C₂-C₄)-alkenyloxy, (C₂-C₆)-alkynyloxy or (C₂-C₄)-haloalkoxy and R_{H}⁴ represents hydrogen or (C₁-C₄)-alkyl or R_{H}³ and R_{H}⁴ together with the directly attached nitrogen atom represent a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.
S16) Active compounds which are used primarily as herbicides but also have safener effects on crop plants, for example (2,4-dichlorophenoxy)acetic acid (2,4-D), (4-chlorophenoxy)acetic acid, (R,S)-2-(4-chloro-o-tolyloxy)propionic acid (mecoprop), 4-(2,4-dichlorophenoxy)butyric acid (2,4-DB), (4-chloro-o-tolyloxy)acetic acid (MCPA), 4-(4-chloro-o-tolyloxy)butyric acid, 4-(4-chlorophenoxy)butyric acid, 3,6-dichloro-2-methoxybenzoic acid (dicamba), 1-(ethoxycarbonyl)ethyl 3,6-dichloro-2-methoxybenzoate (lactidichlor-ethyl).

Some preferred safeners that may be employed in an agricultural composition are: cloquintocet-mexyl, cyprosulfamide, fenchlorazole ethyl ester, isoxadifen-ethyl, mefenpyr-diethyl, fenclorim, cumyluron, S4-1 and S4-5, and particularly preferred safeners are: cloquintocet-mexyl, cyprosulfamide, isoxadifen-ethyl and mefenpyr-diethyl.

Specific fungicides that may be used in an agricultural composition include, for example:
(a) Inhibitors of the ergosterol biosynthesis, including for example cyproconazole, difenoconazole, epoxiconazole, fenbuconazole, fenhexamid, fenpropidin, fenpropimorph, fenpyrazamine, fluquinconazole, flutriafol, hexaconazole, imazalil, imazalil sulfate, ipconazole, ipfentrifluconazole, mefentrifluconazole, metconazole, myclobutanil, paclobutrazol, penconazole, prochloraz, propiconazole, prothioconazole, pyrisoxazole, spiroxamine, tebuconazole, tetraconazole, triadimenol, tridemorph, triticonazole, (1R,2S,5S)-5-(4-chlorobenzyl)-2-(chloromethyl)-2-methyl-1-(1H-1,2,4-triazol-1-ylmethyl)cyclopentanol, (1S,2R,5R)-5-(4-chlorobenzyl)-2-(chloromethyl)-2-methyl-1-(1H-1,2,4-triazol-1-ylmethyl)cyclopentanol, ((2R)-2-(1-chlorocyclopropyl)-4-[(1R)-2,2-dichlorocyclopropyl]-1-(1H-1,2,4-triazol-1-yl)butan-2-ol, (2R)-2-(1-chlorocyclopropyl)-4-[(1S)-2,2-dichlorocyclopropyl]-1-(1H-1,2,4-triazol-1-yl)butan-2-ol, (2R)-2-[4-(4-chlorophenoxy)-2-(trifluoromethyl)phenyl]-1-(1H-1,2,4-triazol-1-yl)propan-2-ol, (2S)-2-(1-chlorocyclopropyl)-4-[(1R)-2,2-dichlorocyclopropyl]-1-(1H-1,2,4-triazol-1-yl)butan-2-ol, (2S)-2-(1-chlorocyclopropyl)-4-[(1S)-2,2-dichlorocyclopropyl]-1-(1H-1,2,4-triazol-1-yl)butan-2-ol, (2S)-2-[4-(4-chlorophenoxy)-2-(trifluoromethyl)phenyl]-1-(1H-1,2,4-triazol-1-yl)propan-2-ol, (R)-[3-(4-chloro-2-fluorophenyl)-5-(2,4-difluorophenyl)-1,2-oxazol-4-yl](pyridin-3-yl)methanol, (S)-[3-(4-chloro-2-fluorophenyl)-5-(2,4-difluorophenyl)-1,2-oxazol-4-yl](pyridin-3-yl)methanol, [3-(4-chloro-2-fluorophenyl)-5-(2,4-difluorophenyl)-1,2-oxazol-4-yl](pyridin-3-yl)methanol, 1-(((2R,4S)-2-[2-chloro-4-(4-chlorophenoxy)phenyl]-4-methyl-1,3-dioxolan-2-yl)methyl)-1H-1,2,4-triazole, 1-({(2S,4S)-2-[2-chloro-4-(4-chlorophenoxy)phenyl]-4-methyl-1,3-dioxolan-2-yl}methyl)-1H-1,2,4-triazole, 1-{[3-(2-chlorophenyl)-2-(2,4-difluorophenyl)oxiran-2-yl]methyl)-1H-1,2,4-triazol-5-yl thiocyanate, 1-{[rel(2R,3R)-3-(2-chlorophenyl)-2-(2,4-difluorophenyl)oxiran-2-yl]methyl)-1H-1,2,4-triazol-5-yl thiocyanate, 1-{[rel(2R,3S)-3-(2-chlorophenyl)-2-(2,4-difluorophenyl)oxiran-2-yl]methyl}-1H-1,2,4-triazol-5-yl thiocyanate, 2-[(2R,4R,5R)-1-(2,4-dichlorophenyl)-5-hydroxy-2,6,6-trimethyl-heptan-4-yl]-2,4-dihydro-3H-1,2,4-triazole-3-thione, 2-[(2R,4R,5S)-1-(2,4-dichlorophenyl)-5-hydroxy-2,6,6-trimethylheptan-4-yl]-2,4-dihydro-3H-1,2,4-triazole-3-thione, 2-[(2R,4S,5R)-1-(2,4-dichlorophenyl)-5-hydroxy-2,6,6-trimethylheptan-4-yl]-2,4-dihydro-3H-1,2,4-triazole-3-thione, 2-[(2R,4S,5S)-1-(2,4-dichlorophenyl)-5-hydroxy-2,6,6-trimethylheptan-4-yl]-2,4-dihydro-3H-1,2,4-triazole-3-thione, 2-[(2S,4R,5R)-1-(2,4-dichlorophenyl)-5-hydroxy-2,6,6-trimethyl-heptan-4-yl]-2,4-dihydro-3H-1,2,4-triazole-3-thione, 2-[(2S,4R,5S)-1-(2,4-dichlorophenyl)-5-hydroxy-2,6,6-trimethylheptan-4-yl]-2,4-dihydro-3H-1,2,4-triazole-3-thione, 2-[(2S,4S,5R)-1-(2,4-dichlorophenyl)-5-hydroxy-2,6,6-trimethylheptan-4-yl]-2,4-dihydro-3H-1,2,4-triazole-3-thione, 2-[(2S,4S,5S)-1-(2,4-dichlorophenyl)-5-hydroxy-2,6,6-trimethylheptan-4-yl]-2,4-dihydro-3H-1,2,4-triazole-3-thione, 2-[1-(2,4-dichlorophenyl)-5-hydroxy-2,6,6-trimethylheptan-4-yl]-2,4-dihydro-3H-1,2,4-triazole-3-thione, 2-[6-(4-bromophenoxy)-2-(trifluoromethyl)-3-pyridyl]-1-(1,2,4-triazol-1-yl)propan-2-ol, 2-[6-(4-chlorophenoxy)-2-(trifluoromethyl)-3-pyridyl]-1-(1,2,4-triazol-1-yl)propan-2-ol, 2-{[3-(2-chlorophenyl)-2-(2,4-difluorophenyl)oxiran-2-yl]methyl}-2,4-dihydro-3H-1,2,4-triazole-3-thione, 2-{[rel(2R,3R)-3-(2-chlorophenyl)-2-(2,4-difluorophenyl)oxiran-2-yl]methyl}-2,4-dihydro-3H-1,2,4-triazole-3-thione, 2-{[rel(2R,3S)-3-(2-chlorophenyl)-2-(2,4-difluorophenyl)-oxiran-2-yl]methyl}-2,4-dihydro-3H-1,2,4-triazole-3-thione, 3-[2-(1-chlorocyclopropyl)-3-(3-chloro-2-fluoro-phenyl)-2-hydroxy-propyl]imidazole-4-carbonitrile, 4-[[6-[rac-(2R)-2-(2,4-difluorophenyl)-1,1-difluoro-2-hydroxy-3-(5-thioxo-4H-1,2,4-triazol-1-yl)propyl]-3-pyridyl]oxy]-benzonitrile, 5-(4-chlorobenzyl)-2-(chloromethyl)-2-methyl-1-(1H-1,2,4-triazol-1-ylmethyl)cyclo-pentanol, 5-(allylsulfanyl)-1-{[3-(2-chlorophenyl)-2-(2,4-difluorophenyl)oxiran-2-yl]methyl}-1H-1,2,4-triazole, 5-(allylsulfanyl)-1-([rel(2R,3R)-3-(2-chlorophenyl)-2-(2,4-difluorophenyl)oxiran-2-yl]methyl)-1H-1,2,4-triazole, 5-(allylsulfanyl)-1-([rel(2R,3S)-3-(2-chlorophenyl)-2-(2,4-difluoro-phenyl)oxiran-2-yl]methyl)-1H-1,2,4-triazole, methyl 2-[2-chloro-4-(4-chlorophenoxy)phenyl]-2-hydroxy-3-(1H-1,2,4-triazol-1-yl)propanoate, N′-(2-chloro-5-methyl-4-phenoxyphenyl)-N-ethyl-N-methylimidoformamide, N′-[2-chloro-4-(2-fluorophenoxy)-5-methylphenyl]-N-ethyl-N-methy-limidoformamide, N′-[5-bromo-6-(2,3-dihydro-1H-inden-2-yloxy)-2-methylpyridin-3-yl]-N-ethyl-N-methylimidoformamide, N′-(4-[(4,5-dichloro-1,3-thiazol-2-yl)oxy]-2,5-dimethylphenyl)-N-ethyl-N-methylimidoformamide, N′-{5-bromo-2-methyl-6-[(1-propoxypropan-2-yl)oxy]pyridin-3-yl}-N-ethyl-N-methylimidoformamide, N′-(5-bromo-6-[(1R)-1-(3,5-difluorophenyl)ethoxy]-2-methyl-pyridin-3-yl)-N-ethyl-N-methylimido-formamide, N′-{5-bromo-6-[(1S)-1-(3,5-difluorophenyl)-ethoxy]-2-methylpyridin-3-yl}-N-ethyl-N-methylimidoformamide, N′-(5-bromo-6-[(cis-4-isopropylcyclohexyl)oxy]-2-methylpyridin-3-yl)-N-ethyl-N-methylimidoformamide, N′-{5-bromo-6-[(trans-4-isopropylcyclohexyl)oxy]-2-methylpyridin-3-yl}-N-ethyl-N-methylimidoformamide, N′-{5-bromo-6-[1-(3,5-difluorophenyl)ethoxy]-2-methylpyridin-3-yl}-N-ethyl-N-methylimidoformamide, N-isopropyl-N′-[5-methoxy-2-methyl-4-(2,2,2-trifluoro-1-hydroxy-1-phenylethyl)phenyl]-N-methylimidoformamide.
(b) Inhibitors of the respiratory chain at complex I or II, including for example benzovindiflupyr, bixafen, boscalid, carboxin, cyclobutrifluram, flubeneteram, fluindapyr, fluopyram, flutolanil, fluxapyroxad, furametpyr, inpyrfluxam, isofetamid, isoflucypram, isopyrazam, penflufen, penthiopyrad, pydiflumetofen, pyrapropoyne, pyraziflumid, sedaxane,
1,3-dimethyl-N-(1,1,3-trimethyl-2,3-dihydro-1H-inden-4-yl)-1H-pyrazole-4-carboxamide, 1,3-dimethyl-N-[(3R)-1,1,3-trimethyl-2,3-dihydro-1H-inden-4-yl]-1H-pyrazole-4-carboxamide, 1,3-dimethyl-N-[(3S)-1,1,3-trimethyl-2,3-dihydro-1H-inden-4-yl]-1H-pyrazole-4-carboxamide, 1-methyl-3-(trifluoromethyl)-N-[2′-(trifluoromethyl)biphenyl-2-yl]-1H-pyrazole-4-carboxamide, 2-fluoro-6-(trifluoromethyl)-N-(1,1,3-trimethyl-2,3-dihydro-1H-inden-4-yl)benzamide, 3-(difluoromethyl)-1-methyl-N-(1,1,3-trimethyl-2,3-dihydro-1H-inden-4-yl)-1H-pyrazole-4-carboxamide, 3-(difluoromethyl)-1-methyl-N-[(3S)-1,1,3-trimethyl-2,3-dihydro-1H-inden-4-yl]-1H-pyrazole-4-carboxamide, 3-(difluoromethyl)-N-[(3R)-7-fluoro-1,1,3-trimethyl-2,3-dihydro-1H-inden-4-yl]-1-methyl-1H-pyrazole-4-carboxamide, 3-(difluoromethyl)-N-[(3S)-7-fluoro-1,1,3-trimethyl-2,3-dihydro-1H-inden-4-yl]-1-methyl-1H-pyrazole-4-carboxamide, 5,8-difluoro-N-[2-(2-fluoro-4-{[4-(trifluoromethyl)pyridin-2-yl]oxy}phenyl)ethyl]quinazolin-4-amine, N-[(1R,4S)-9-(dichloro-methylene)-1,2,3,4-tetrahydro-1,4-methanonaphthalen-5-yl]-3-(difluoromethyl)-1-methyl-1H-pyrazole-4-carboxamide, N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, N-[1-(2,4-dichlorophenyl)-1-methoxypropan-2-yl]-3-(difluoromethyl)-1-methyl-1H-pyrazole-4-carboxamide, N-[rac-(1S,2S)-2-(2,4-dichlorophenyl)cyclobutyl]-2-(trifluoromethyl)nicotinamide.
(c) Inhibitors of the respiratory chain at complex III, including for example ametoctradin, amisulbrom, azoxystrobin, coumethoxystrobin, coumoxystrobin, cyazofamid, dimoxystrobin, enoxastrobin, famoxadone, fenamidone, fenpicoxamid, florylpicoxamid, flufenoxystrobin, fluoxastrobin, kresoxim-methyl, mandestrobin, metominostrobin, metyltetraprole, orysastrobin, picoxystrobin, pyraclostrobin, pyrametostrobin, pyraoxystrobin, trifloxystrobin, (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (2E,3Z)-5-{[1-(4-chlorophenyl)-1H-pyrazol-3-yl]oxy}-2-(methoxyimino)-N,3-dimethylpent-3-enamide, (2R)-2-{2-[(2,5-dimethylphenoxy)methyl]phenyl}-2-methoxy-N-methylacetamide, (2S)-2-(2-[(2,5-dimethylphenoxy)methyl]phenyl)-2-methoxy-N-methylacetamide, N-(3-ethyl-3,5,5-trimethylcyclohexyl)-3-formamido-2-hydroxybenzamide, (2E,3Z)-5-{[1-(4-chloro-2-fluorophenyl)-1H-pyrazol-3-yl]oxy}-2-(methoxyimino)-N,3-dimethylpent-3-enamide, methyl (5-[3-(2,4-dimethylphenyl)-1H-pyrazol-1-yl]-2-methylbenzyl)carbamate.
(d) Inhibitors of the mitosis and cell division, including for example carbendazim, diethofencarb, ethaboxam, fluopicolide, fluopimomide, metrafenone, pencycuron, pyridachlometyl, pyriofenone (chlazafenone), thiabendazole, thiophanate-methyl, zoxamide, 3-chloro-5-(4-chlorophenyl)-4-(2,6-difluorophenyl)-6-methylpyridazine, 3-chloro-5-(6-chloropyridin-3-yl)-6-methyl-4-(2,4,6-trifluorophenyl)pyridazine, 4-(2-bromo-4-fluorophenyl)-N-(2,6-difluorophenyl)-1,3-dimethyl-1H-pyrazol-5-amine, 4-(2-bromo-4-fluorophenyl)-N-(2-bromo-6-fluorophenyl)-1,3-dimethyl-1H-pyrazol-5-amine, 4-(2-bromo-4-fluorophenyl)-N-(2-bromophenyl)-1,3-dimethyl-1H-pyrazol-5-amine, 4-(2-bromo-4-fluorophenyl)-N-(2-chloro-6-fluorophenyl)-1,3-dimethyl-1H-pyrazol-5-amine, 4-(2-bromo-4-fluorophenyl)-N-(2-chlorophenyl)-1,3-dimethyl-1H-pyrazol-5-amine, 4-(2-bromo-4-fluorophenyl)-N-(2-fluorophenyl)-1,3-dimethyl-1H-pyrazol-5-amine, 4-(2-chloro-4-fluorophenyl)-N-(2,6-difluorophenyl)-1,3-dimethyl-1H-pyrazol-5-amine, 4-(2-chloro-4-fluorophenyl)-N-(2-chloro-6-fluorophenyl)-1,3-dimethyl-1H-pyrazol-5-amine, 4-(2-chloro-4-fluorophenyl)-N-(2-chlorophenyl)-1,3-dimethyl-1H-pyrazol-5-amine, 4-(2-chloro-4-fluorophenyl)-N-(2-fluorophenyl)-1,3-dimethyl-1H-pyrazol-5-amine, 4-(4-chlorophenyl)-5-(2,6-difluorophenyl)-3,6-dimethylpyridazine, N-(2-bromo-6-fluorophenyl)-4-(2-chloro-4-fluorophenyl)-1,3-dimethyl-1H-pyrazol-5-amine, N-(2-bromophenyl)-4-(2-chloro-4-fluorophenyl)-1,3-dimethyl-1H-pyrazol-5-amine, N-(4-chloro-2,6-difluorophenyl)-4-(2-chloro-4-fluorophenyl)-1,3-dimethyl-1H-pyrazol-5-amine.
(e) Compounds capable to have a multisite action, including for example bordeaux mixture, captafol, captan, chlorothalonil, copper hydroxide, copper naphthenate, copper oxide, copper oxychloride, copper(2+) sulfate, dithianon, dodine, folpet, mancozeb, maneb, metiram, metiram zinc, oxine-copper, propineb, sulfur and sulfur preparations including calcium polysulfide, thiram, zineb, ziram, and 6-ethyl-5,7-dioxo-6,7-dihydro-5H-pyrrolo[3′,4′:5,6][1,4]dithiino[2,3-c][1,2]thiazole-3-carbonitrile.
(f) Compounds capable to induce a host defence, including for example acibenzolar-S-methyl, fosetyl-aluminium, fosetyl-calcium, fosetyl-sodium, isotianil, phosphorous acid and its salts, probenazole, tiadinil.
(g) Inhibitors of the amino acid and/or protein biosynthesis, including for example cyprodinil, kasugamycin, kasugamycin hydrochloride hydrate, oxytetracycline, pyrimethanil.
(h) Inhibitors of the ATP production, including for example silthiofam.
(i) Inhibitors of the cell wall synthesis, including for example benthiavalicarb, dimethomorph, flumorph, iprovalicarb, mandipropamid, pyrimorph, valifenalate, (2E)-3-(4-tert-butylphenyl)-3-(2-chloropyridin-4-yl)-1-(morpholin-4-yl)prop-2-en-1-one, (2Z)-3-(4-tert-butylphenyl)-3-(2-chloropyridin-4-yl)-1-(morpholin-4-yl)prop-2-en-1-one.
(j) Inhibitors of the lipid synthesis or transport, or membrane synthesis, including for example fluoxapiprolin, natamycin, oxathiapiprolin, propamocarb, propamocarb hydrochloride, propamocarb-fosetylate, tolclofos-methyl, 1-(4-(4-[(5R)-5-(2,6-difluorophenyl)-4,5-dihydro-1,2-oxazol-3-yl]-1,3-thiazol-2-yl)piperidin-1-yl)-2-[5-methyl-3-(trifluoromethyl)-1H-pyrazol-1-yl]ethanone, 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, 3-[2-(1-{[5-methyl-3-(trifluoromethyl)-1H-pyrazol-1-yl]acetyl}piperidin-4-yl)-1,3-thiazol-4-yl]-1,5-dihydro-2,4-benzodioxepin-6-yl methanesulfonate, 9-fluoro-3-[2-(1-{[5-methyl-3-(trifluoromethyl)-1H-pyrazol-1-yl]acetyl}piperidin-4-yl)-1,3-thiazol-4-yl]-1,5-dihydro-2,4-benzodioxepin-6-yl methanesulfonate, 3-[2-(1-{[3,5-bis(difluoromethyl)-1H-pyrazol-1-yl]acetyl}piperidin-4-yl)-1,3-thiazol-4-yl]-1,5-dihydro-2,4-benzodioxepin-6-yl methanesulfonate, 3-[2-(1-{[3,5-bis(difluoromethyl)-1H-pyrazol-1-yl]acetyl}piperidin-4-yl)-1,3-thiazol-4-yl]-9-fluoro-1,5-dihydro-2,4-benzodioxepin-6-yl methanesulfonate.
(k) Inhibitors of the melanin biosynthesis, including for example tolprocarb, tricyclazole.
(l) Inhibitors of the nucleic acid synthesis, including for example benalaxyl, benalaxyl-M (kiralaxyl), metalaxyl, and metalaxyl-M (mefenoxam).
(m)Inhibitors of the signal transduction, including for example fludioxonil, iprodione, procymidone, proquinazid, quinoxyfen, vinclozolin.
(n) Compounds capable to act as an uncoupler, including for example fluazinam, meptyldinocap.
(o) Additional fungicidal compounds, including for example abscisic acid, aminopyrifen, benthiazole, bethoxazin, capsimycin, carvone, chinomethionat, cufraneb, cyflufenamid, cymoxanil, cyprosulfamide, dipymetitrone, flutianil, ipflufenoquin, methyl isothiocyanate, mildiomycin, nickel dimethyldithiocarbamate, nitrothal-isopropyl, oxyfenthiin, pentachlorophenol and salts, picarbutrazox, quinofumelin, D-tagatose, tebufloquin, tecloftalam, tolnifanide, 2-(6-benzylpyridin-2-yl)quinazoline, 2-[6-(3-fluoro-4-methoxyphenyl)-5-methylpyridin-2-yl]quinazoline, 2-phenylphenol and salts, 4-amino-5-fluoropyrimidin-2-ol (tautomeric form: 4-amino-5-fluoropyrimidin-2(1H)-one), 4-oxo-4-[(2-phenylethyl)amino]butanoic acid, 5-amino-1,3,4-thiadiazole-2-thiol, 5-chloro-N′-phenyl-N′-(prop-2-yn-1-yl)thiophene-2-sulfonohydrazide, 5-fluoro-2-[(4-fluorobenzyl)oxy]-pyrimidin-4-amine, 5-fluoro-2-[(4-methylbenzyl)oxy]pyrimidin-4-amine, 5-fluoro-4-imino-3-methyl-1-[(4-methylphenyl)sulfonyl]-3,4-dihydropyrimidin-2(1H)-one, but-3-yn-1-yl{6-[({[(Z)-(1-methyl-1H-tetrazol-5-yl)(phenyl)methylene]amino}oxy)methyl]pyridin-2-yl)carbamate, ethyl (2Z)-3-amino-2-cyano-3-phenylacrylate, phenazine-1-carboxylic acid, propyl 3,4,5-trihydroxybenzoate, (15.041) quinolin-8-ol, quinolin-8-ol sulfate (2:1), 1-(4,5-dimethyl-1H-benzimidazol-1-yl)-4,4-difluoro-3,3-dimethyl-3,4-dihydroisoquinoline, 1-(5-(fluoromethyl)-6-methyl-pyridin-3-yl)-4,4-difluoro-3,3-dimethyl-3,4-dihydroisoquinoline, 1-(5,6-dimethylpyridin-3-yl)-4,4-difluoro-3,3-dimethyl-3,4-dihydroisoquinoline, 1-(6-(difluoromethyl)-5-methoxy-pyridin-3-yl)-4,4-difluoro-3,3-dimethyl-3,4-dihydroisoquinoline, 1-(6-(difluoromethyl)-5-methyl-pyridin-3-yl)-4,4-difluoro-3,3-dimethyl-3,4-dihydroisoquinoline, 1-(6,7-dimethylpyrazolo[1,5-a]pyridin-3-yl)-4,4-difluoro-3,3-dimethyl-3,4-dihydroisoquinoline, 2-(2-fluoro-6-[(8-fluoro-2-methylquinolin-3-yl)oxy]phenyl)propan-2-ol, 3-(4,4,5-trifluoro-3,3-dimethyl-3,4-dihydroisoquinolin-1-yl)quinoline, 3-(4,4-difluoro-3,3-dimethyl-3,4-dihydroisoquinolin-1-yl)-8-fluoroquinoline, 3-(4,4-difluoro-5,5-dimethyl-4,5-dihydrothieno[2,3-c]pyridin-7-yl)quinoline, 3-(5-fluoro-3,3,4,4-tetramethyl-3,4-dihydroisoquinolin-1-yl)quinoline, 5-bromo-1-(5,6-dimethylpyridin-3-yl)-3,3-dimethyl-3,4-dihydroisoquinoline, 8-fluoro-3-(5-fluoro-3,3,4,4-tetramethyl-3,4-dihydroisoquinolin-1-yl)-quinoline, 8-fluoro-3-(5-fluoro-3,3-dimethyl-3,4-dihydroisoquinolin-1-yl)-quinoline, 8-fluoro-N-(4,4,4-trifluoro-2-methyl-1-phenylbutan-2-yl)quinoline-3-carboxamide, 8-fluoro-N-[(2S)-4,4,4-trifluoro-2-methyl-1-phenylbutan-2-yl]quinoline-3-carboxamide, 9-fluoro-2,2-dimethyl-5-(quinolin-3-yl)-2,3-dihydro-1,4-benzoxazepine, (15.060)N-(2,4-dimethyl-1-phenylpentan-2-yl)-8-fluoroquinoline-3-carboxamide, N-[(2S)-2,4-dimethyl-1-phenylpentan-2-yl]-8-fluoroquinoline-3-carboxamide, 1,1-diethyl-3-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, 1-[[3-fluoro-4-(5-(trifluoromethyl)-1,2,4-oxadiazol-3-yl)phenyl]methyl]azepan-2-one, 1-[[4-[5-(trifluoromethyl)-1,2,4-oxadiazol-3-yl]phenyl]methyl]piperidin-2-one, 1-methoxy-1-methyl-3-[[4-[5-(trifluoromethyl)-1,2,4-oxadiazol-3-yl]phenyl]methyl]urea, 1-methoxy-3-methyl-1-[[4-[5-(trifluoromethyl)-1,2,4-oxadiazol-3-yl]phenyl]methyl]urea, 1-methoxy-3-methyl-1-[[4-[5-(trifluoromethyl)-1,2,4-oxadiazol-3-yl]phenyl]methyl]urea, 2,2-difluoro-N-methyl-2-[4-[5-(trifluoromethyl)-1,2,4-oxadiazol-3-yl]phenyl]acetamide, 3,3-dimethyl-1-[[4-[5-(trifluoromethyl)-1,2,4-oxadiazol-3-yl]phenyl]methyl]piperidin-2-one, 3-ethyl-1-methoxy-1-[[4-[5-(trifluoromethyl)-1,2,4-oxadiazol-3-yl]phenyl]methyl]urea, 4,4-dimethyl-1-[[4-[5-(trifluoromethyl)-1,2,4-oxadiazol-3-yl]phenyl]methyl]pyrrolidin-2-one, 4,4-dimethyl-2-[[4-[5-(trifluoromethyl)-1,2,4-oxadiazol-3-yl]phenyl]methyl]isoxazolidin-3-one, 4-[5-(trifluoromethyl)-1,2,4-oxadiazol-3-yl]phenyl dimethylcarbamate, 5,5-dimethyl-2-[[4-[5-(trifluoromethyl)-1,2,4-oxadiazol-3-yl]phenyl]methyl]isoxazolidin-3-one, 5-methyl-1-[[4-[5-(trifluoromethyl)-1,2,4-oxadiazol-3-yl]phenyl]methyl]pyrrolidin-2-one, ethyl 1-(4-[5-(trifluoromethyl)-1,2,4-oxadiazol-3-yl]benzyl)-1H-pyrazole-4-carboxylate, methyl (4-[5-(trifluoromethyl)-1,2,4-oxadiazol-3-yl]phenyl)carbamate, N-(1-methylcyclopropyl)-4-[5-(trifluoromethyl)-1,2,4-oxadiazol-3-yl]benzamide, N-(2,4-difluorophenyl)-4-[5-(trifluoromethyl)-1,2,4-oxadiazol-3-yl]benzamide, N-(2-fluorophenyl)-4-[5-(trifluoromethyl)-1,2,4-oxadiazol-3-yl]benzamide, N,2-dimethoxy-N-[[4-[5-(trifluoromethyl)-1,2,4-oxadiazol-3-yl]phenyl]methyl]propanamide, N,N-dimethyl-1-(4-[5-(trifluoromethyl)-1,2,4-oxadiazol-3-yl]benzyl)-1H-1,2,4-triazol-3-amine, N-[(E)-methoxyiminomethyl]-4-[5-(trifluoromethyl)-1,2,4-oxadiazol-3-yl]benzamide, N-[(E)-N-methoxy-C-methyl-carbonimidoyl]-4-[5-(trifluoromethyl)-1,2,4-oxadiazol-3-yl]benzamide, N—[(Z)-methoxyiminomethyl]-4-[5-(trifluoromethyl)-1,2,4-oxadiazol-3-yl]benzamide, N—[(Z)—N-methoxy-C-methyl-carbonimidoyl]-4-[5-(trifluoromethyl)-1,2,4-oxadiazol-3-yl]benzamide, N-[[2,3-difluoro-4-[5-(trifluoromethyl)-1,2,4-oxadiazol-3-yl]phenyl]methyl]-3,3,3-trifluoro-propanamide, N-[[4-[5-(trifluoromethyl)-1,2,4-oxadiazol-3-yl]phenyl]methyl]propanamide, (15.090)N-[4-[5-(trifluoromethyl)-1,2,4-oxadiazol-3-yl]phenyl]cyclopropanecarboxamide, N-(2,3-difluoro-4-[5-(trifluoromethyl)-1,2,4-oxadiazol-3-yl]benzyl)butanamide, N-{4-[5-(trifluoromethyl)-1,2,4-oxadiazol-3-yl]benzyl}cyclopropanecarboxamide, N-{4-[5-(trifluoromethyl)-1,2,4-oxadiazol-3-yl]phenyl}propanamide, N-allyl-N-[[4-[5-(trifluoromethyl)-1,2,4-oxadiazol-3-yl]phenyl]methyl]acetamide, N-allyl-N-[[4-[5-(trifluoromethyl)-1,2,4-oxadiazol-3-yl]phenyl]methyl]propanamide, N-ethyl-2-methyl-N-[[4-[5-(trifluoromethyl)-1,2,4-oxadiazol-3-yl]phenyl]methyl]propanamide, N-methoxy-N-[[4-[5-(trifluoromethyl)-1,2,4-oxadiazol-3-yl]phenyl]methyl]cyclopropanecarboxamide, N-methyl-4-[5-(trifluoromethyl)-1,2,4-oxadiazol-3-yl]benzamide, N-methyl-4-[5-(trifluoromethyl)-1,2,4-oxadiazol-3-yl]benzenecarbothioamide, and N-methyl-N-phenyl-4-[5-(trifluoromethyl)-1,2,4-oxadiazol-3-yl]benzamide.

Specific insecticides that may be used in an agricultural composition include, for example:
(a) Acetylcholinesterase (AChE) inhibitors, including for example carbamates selected from alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb, or organophosphates selected from acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.
(b) GABA-gated chloride channel blockers, including for example cyclodiene-organochlorines selected from chlordane and endosulfan, or phenylpyrazoles (fiproles) selected from ethiprole and fipronil.
(c) Sodium channel modulators, including for example pyrethroids selected from acrinathrin, allethrin, d-cis-trans allethrin, d-trans allethrin, bifenthrin, bioallethrin, bioallethrin s-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans-isomer], deltamethrin, empenthrin [(EZ)-(1R)-isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans-isomer], prallethrin, pyrethrins (pyrethrum), resmethrin, silafluofen, tefluthrin, tetramethrin, tetramethrin [(1R)-isomer)], tralomethrin and transfluthrin, or DDT or methoxychlor.
(d) Nicotinic acetylcholine receptor (nAChR) competitive modulators, including for example neonicotinoids selected from acetamiprid, clothianidin, dinotefuran, imidacloprid, nitenpyram, thiacloprid and thiamethoxam, or nicotine, or sulfoximines selected from sulfoxaflor, or butenolids selected from flupyradifurone, or mesoionics such as triflumezopyrim.
(e) Nicotinic acetylcholine receptor (nAChR) allosteric modulators (Site I), including for example spinosyns selected from spinetoram and spinosad.
(f) Glutamate-gated chloride channel (GIuCi) allosteric modulators, including for example avermectins/milbemycins selected from abamectin, emamectin benzoate, lepimectin and milbemectin.
(g) Juvenile hormone mimics, including for example juvenile hormone analogues selected from hydroprene, kinoprene and methoprene, or fenoxycarb or pyriproxyfen.
(h) Miscellaneous non-specific (multi-site) inhibitors, including for example alkyl halides selected from methyl bromide and other alkyl halides, or chloropicrine or sulphuryl fluoride or borax or tartar emetic or methyl isocyanate generators selected from diazomet and metam.
(i) Chordotonal organ TRPV channel modulators, including for example pyridine azomethanes selected from pymetrozine and pyrifluquinazone, or pyropenes selected from afidopyropen.
(j) Mite growth inhibitors affecting CHS1 selected from clofentezine, hexythiazox, diflovidazin and etoxazole.
(k) Microbial disruptors of the insect gut membranes selected from Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and B.t. plant proteins selected from Cry1Ab, Cry lAc, Cry1Fa, Cry1A.105, Cry2Ab, Vip3A, mCry3A, Cry3Ab, Cry3Bb and Cry34Ab1/35Ab1.
(l) Inhibitors of mitochondrial ATP synthase, including for example ATP disruptors selected from diafenthiuron, or organotin compounds selected from azocyclotin, cyhexatin and fenbutatin oxide, or propargite or tetradifon.
(m) Uncouplers of oxidative phosphorylation via disruption of the proton gradient selected from chlorfenapyr, DNOC and sulfluramid.
(n) Nicotinic acetylcholine receptor channel blockers selected from bensultap, cartap hydrochloride, thiocylam and thiosultap-sodium.
(o) Inhibitors of chitin biosynthesis affecting CHS1, including for example benzoylureas selected from bistrifluron, chlorfluazuron, diflubenzuron, flucycloxuron, flufenoxuron, hexaflumuron, lufenuron, novaluron, noviflumuron, teflubenzuron and triflumuron.
(p) Inhibitors of chitin biosynthesis, type 1 such as buprofezin.
(q) Moulting disruptor (in particular for Diptera, i.e. dipterans) such as cyromazine.
(r) Ecdysone receptor agonists, including for example diacylhydrazines selected from chromafenozide, halofenozide, methoxyfenozide and tebufenozide.
(s) Octopamine receptor agonists such as amitraz.
(t) Mitochondrial complex III electron transport inhibitors selected from hydramethylnone, acequinocyl, fluacrypyrim and bifenazate.
(u) Mitochondrial complex I electron transport inhibitors, including for example METI acaricides and insecticides selected from fenazaquin, fenpyroximate, pyrimidifen, pyridaben, tebufenpyrad and tolfenpyrad, or rotenone (Derris).
(v) Voltage-dependent sodium channel blockers, including for example oxadiazines selected from indoxacarb, or semicarbazones selected from metaflumizone.
(w)Inhibitors of acetyl CoA carboxylase, including for example tetronic and tetramic acid derivatives selected from spirodiclofen, spiromesifen, spiropidion and spirotetramat.
(x) Mitochondrial complex IV electron transport inhibitors, including for example phosphides selected from aluminium phosphide, calcium phosphide, phosphine and zinc phosphide, or cyanides selected from calcium cyanide, potassium cyanide and sodium cyanide.
(y) Mitochondrial complex II electron transport inhibitors, including for example beta-ketonitrile derivatives selected from cyenopyrafen and cyflumetofen, or carboxanilides selected from pyflubumide.
(z) Ryanodine receptor modulators, including for example diamides selected from chlorantraniliprole, cyantraniliprole, cyclaniliprole, flubendiamide and tetraniliprole.
(aa) Chordotonal organ Modulators (with undefined target site) such as flonicamid.
(bb) GABA-gated chlorid channel allosteric modulators, including for example meta-diamides selected from broflanilide, or isoxazoles selected from fluxametamide.
(cc) Baculovisuses, preferably Granuloviruses (GVs) selected from Cydia pomonella GV and Thaumatotibia leucotreta (GV), or Nucleopolyhedroviruses (NPVs) selected from Anticarsia gemmatalis MNPV and Helicoverpa armigera NPV.
(dd) Nicotinic acetylcholine receptor allosteric modulators (Site II) such as GS-omega/kappa HXTX-Hvla peptide.
(ee) Additional insecticidal compounds selected from Acynonapyr, Afoxolaner, Azadirachtin, Benclothiaz, Benzoximate, Benzpyrimoxan, Bromopropylate, Chinomethionat, Chloroprallethrin, Cryolite, Cyclobutrifluram, Cycloxaprid, Cyetpyrafen, Cyhalodiamide, Cyproflanilide (CAS 2375110-88-4), Dicloromezotiaz, Dicofol, Dimpropyridaz, epsilon-Metofluthrin, epsilon-Momfluthrin, Flometoquin, Fluazaindolizine, Flucypyriprole (CAS 1771741-86-6), Fluensulfone, Flufenerim, Flufenoxystrobin, Flufiprole, Fluhexafon, Fluopyram, Flupyrimin, Fluralaner, Fufenozide, Flupentiofenox, Guadipyr, Heptafluthrin, Imidaclothiz, Iprodione, Isocycloseram, kappa-Bifenthrin, kappa-Tefluthrin, Lotilaner, Meperfluthrin, Nicofluprole (CAS 1771741-86-6), Oxazosulfyl, Paichongding, Pyridalyl, Pyrifluquinazon, Pyriminostrobin, Sarolaner, Spidoxamat, Spirobudiclofen, Tetramethylfluthrin, Tetrachlorantraniliprole, Tigolaner, Tioxazafen, Thiofluoximate, Tyclopyrazoflor, Iodomethane; furthermore preparations based on Bacillusfirmus (1-1582, Votivo) and azadirachtin (BioNeem), and also the following compounds: 1-{2-fluoro-4-methyl-5-[(2,2,2-trifluoroethyl)sulphinyl]phenyl}-3-(trifluoromethyl)-1H-1,2,4-triazole-5-amine (as described, e.g., in WO2006/043635)(CAS 885026-50-6), 2-chloro-N-[2-{1-[(2E)-3-(4-chlorophenyl)prop-2-en-1-yl]piperidin-4-yl}-4-(trifluoromethyl)phenyl]isonicotinamide (as described, e.g., in WO2006/003494), 3-(4-chloro-2,6-dimethylphenyl)-4-hydroxy-8-methoxy-1,8-diazaspiro[4.5]dec-3-en-2-one (known from WO 2010052161), 3-(4-chloro-2,6-dimethylphenyl)-8-methoxy-2-oxo-1,8-diazaspiro[4.5]dec-3-en-4-yl ethyl carbonate (known from EP2647626), PF1364 (as described, e.g., in JP2010/018586), (3E)-3-[1-[(6-chloro-3-pyridyl)methyl]-2-pyridylidene]-1,1,1-trifluoro-propan-2-one (as described, e.g., in WO2013/144213), N-[3-(benzylcarbamoyl)-4-chlorophenyl]-1-methyl-3-(pentafluoroethyl)-4-(trifluoromethyl)-1H-pyrazole-5-carboxamide (as described, e.g., in WO2010/051926), 5-bromo-4-chloro-N-[4-chloro-2-methyl-6-(methylcarbamoyl)phenyl]-2-(3-chloro-2-pyridyl)pyrazole-3-carboxamide (known from CN103232431), 4-[5-(3,5-dichlorophenyl)-4,5-dihydro-5-(trifluoromethyl)-3-isoxazolyl]-2-methyl-N-(cis-1-oxido-3-thietanyl)-benzamide, 4-[5-(3,5-dichlorophenyl)-4,5-dihydro-5-(trifluoromethyl)-3-isoxazolyl]-2-methyl-N-(trans-1-oxido-3-thietanyl)-benzamide and 4-[(5S)-5-(3,5-dichlorophenyl)-4,5-dihydro-5-(trifluoromethyl)-3-isoxazolyl]-2-methyl-N-(cis-1-oxido-3-thietanyl)benzamide (known from WO 2013/050317 A1), N-[3-chloro-1-(3-pyridinyl)-1H-pyrazol-4-yl]-N-ethyl-3-[(3,3,3-trifluoropropyl)sulfinyl]-propanamide, (+)-N-[3-chloro-1-(3-pyridinyl)-1H-pyrazol-4-yl]-N-ethyl-3-[(3,3,3-trifluoropropyl)sulfinyl]-propanamide and (−)-N-[3-chloro-1-(3-pyridinyl)-1H-pyrazol-4-yl]-N-ethyl-3-[(3,3,3-trifluoropropyl)sulfinyl]-propanamide (as described, e.g., in WO 2013/162715 A2, WO 2013/162716 A2, US 2014/0213448 A1), 5-[[(2E)-3-chloro-2-propen-1-yl]amino]-1-[2,6-dichloro-4-(trifluoromethyl)phenyl]-4-[(trifluoromethyl)sulfinyl]-1H-pyrazole-3-carbonitrile (as described, e.g., in CN 101337937 A), 3-bromo-N-[4-chloro-2-methyl-6-[(methylamino)thioxomethyl]phenyl]-1-(3-chloro-2-pyridinyl)-1H-pyrazole-5-carboxamide, (Liudaibenjiaxuanan, as described, e.g., in CN 103109816 A); N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide (as described, e.g., in WO 2012/034403 A1), N-[2-(5-amino-1,3,4-thiadiazol-2-yl)-4-chloro-6-methylphenyl]-3-bromo-1-(3-chloro-2-pyridinyl)-1H-pyrazole-5-carboxamide (as described, e.g., in WO 2011/085575 A1), 4-[3-[2,6-dichloro-4-[(3,3-dichloro-2-propen-1-yl)oxy]phenoxy]propoxy]-2-methoxy-6-(trifluoromethyl)-pyrimidine (known from CN 101337940 A); (2E)- and 2(Z)-2-[2-(4-cyanophenyl)-1-[3-(trifluoromethyl)phenyl]ethylidene]-N-[4-(difluoromethoxy)phenyl]-hydrazinecarboxamide (as described, e.g., in CN 101715774 A); 3-(2,2-dichloroethenyl)-2,2-dimethyl-4-(1H-benzimidazol-2-yl)phenyl-cyclopropanecarboxylic acid ester (as described, e.g., in CN 103524422 A); (4aS)-7-chloro-2,5-dihydro-2-[[(methoxycarbonyl)[4-[(trifluoromethyl) thio]phenyl]amino]carbonyl]-indeno[1,2-e][1,3,4]oxadiazine-4a(3H)-carboxylic acid methyl ester (as described, e.g., in CN 102391261 A); 6-deoxy-3-O-ethyl-2,4-di-O-methyl-, 1-[N-[4-[1-[4-(1,1,2,2,2-pentafluoroethoxy)phenyl]-1H-1,2,4-triazol-3-yl]phenyl]carbamate]-α-L-mannopyranose (as described, e.g., in US 2014/0275503 A1); 8-(2-cyclopropylmethoxy-4-trifluoromethyl-phenoxy)-3-(6-trifluoromethyl-pyridazin-3-yl)-3-aza-bicyclo[3.2.1]octane, (8-anti)-8-(2-cyclopropylmethoxy-4-trifluoromethyl-phenoxy)-3-(6-trifluoromethyl-pyridazin-3-yl)-3-aza-bicyclo[3.2.1]octane, (8-syn)-8-(2-cyclopropylmethoxy-4-trifluoromethyl-phenoxy)-3-(6-trifluoromethyl-pyridazin-3-yl)-3-aza-bicyclo[3.2.1]octane (as described, e.g., in WO 2007040280 A1, WO 2007040282 A1), N-[4-(aminothioxomethyl)-2-methyl-6-[(methylamino)carbonyl]phenyl]-3-bromo-1-(3-chloro-2-pyridinyl)-1H-pyrazole-5-carboxamide (as described, e.g., in CN 103265527 A), 3-(4-chloro-2,6-dimethylphenyl)-8-methoxy-1-methyl-1,8-diazaspiro[4.5]decane-2,4-dione (as described, e.g., in WO 2014/187846 A1), 3-(4-chloro-2, 6-dimethylphenyl)-8-methoxy-1-methyl-2-oxo-1,8-diazaspiro[4.5]dec-3-en-4-yl-carbonic acid ethyl ester (as described, e.g., in WO 2010/066780 A1, WO 2011151146 A1), N-[1-(2,6-difluorophenyl)-1H-pyrazol-3-yl]-2-(trifluoromethyl)benzamide (as described, e.g., in WO 2014/053450 A1), N-[2-(2,6-difluorophenyl)-2H-1,2,3-triazol-4-yl]-2-(trifluoromethyl) benzamide (as described, e.g., in WO 2014/053450 A1), N-[1-(3,5-difluoro-2-pyridinyl)-1H-pyrazol-3-yl]-2-(trifluoromethyl)benzamide (as described, e.g., in WO 2014/053450 A1), (3R)-3-(2-chloro-5-thiazolyl)-2,3-dihydro-8-methyl-5,7-dioxo-6-phenyl-5H-thiazolo[3,2-a]pyrimidinium inner salt (as described, e.g., in WO 2018/177970 A1); 3-(2-chloro-5-thiazolyl)-2,3-dihydro-8-methyl-5,7-dioxo-6-phenyl-5H-thiazolo[3,2-a]pyrimidinium inner salt (as described, e.g., in WO 2018/177970 A1); N-[3-chloro-1-(3-pyridinyl)-1H-pyrazol-4-yl]-2-(methylsulfonyl)-propanamide (as described, e.g., in WO 2019/236274 A1), N-[2-bromo-4-[1,2,2,2-tetrafluoro-1-(trifluoromethvl)ethyl]-6-(trifluoromethyl)phenyl]-2-fluoro-3-[(4-fluorobenzoyl)amino]-benzamide (as described, e.g., in WO 2019059412 A1).

A modified or transgenic corn plant or plant part, one or more modified or transgenic corn plants or plant parts, or plurality of modified or transgenic corn plants or plant parts, which may be further planted or grown in a greenhouse or an agricultural field or soil, that comprise(s) any mutation, edit or other genetic modification of a brachytic locus or gene, such as a brachytic2 (br2) locus or gene, including any mutant, edited or modified allele (collectively any “mutant allele”) of the brachytic or brachytic2 (br2) gene or locus as described above in connection with applications of an agricultural composition, and which may be homozygous, bi-allelic or heterozygous for one or more mutant allele(s) of the brachytic or brachytic2 (br2) gene or locus, may further comprise one or more additional transgenic event(s) conferring an additional beneficial trait(s) to the modified or transgenic corn plant, including but not limited to, pest resistance, water use efficiency, yield performance, drought tolerance, seed quality, improved nutritional quality, hybrid seed production, and herbicide tolerance, in which the trait is measured with respect to a plant lacking such trait. Examples of such advantageous and/or useful traits may include better plant growth, vigor, stress tolerance, standability, lodging resistance, nutrient uptake, plant nutrition, and/or yield, in particular improved growth, increased tolerance to high or low temperatures, increased tolerance to drought or to levels of water or soil salinity, enhanced flowering performance, easier harvesting, accelerated ripening, higher yields, higher quality and/or a higher nutritional value of the harvested products, better storage life and/or processability of the harvested products, increased resistance against animal and/or microbial pests, such as against insects, arachnids, nematodes, mites, slugs and snails, and increased resistance against phytopathogenic fungi, bacteria and/or viruses. Examples of transgenic or other events providing a beneficial trait to a corn plant may include any of the events in Table 1 below.

TABLE 1
Events in Corn for Additional Beneficial Traits

| | | Deposit Information or | Relevant Patent publication |
|---|---|---|---|
| Event Name | Trait(s) | commercial availability | or regulatory dossier |
| Event 32316 | insect control-herbicide | ATCC PTA-11507 | WO2011/084632 |
| | tolerance | | |
| Event 3272 | quality trait | ATCC PTA-9972 | WO2006/098952 or US-A |
| | | | 2006-230473 |
| Event 40416 | insect control - herbicide | ATCC PTA-11508 | WO2011/075593 |
| | tolerance | | |
| Event 4114 | insect control-herbicide | ATCC PTA-11506 | W02011/084621 |
| | tolerance | | |
| Event 43A47 | insect control - herbicide | ATCC PTA-11509 | WO2011/075595 |
| | tolerance | | |
| Event 5307 | insect control | ATCC PTA-9561 | WO2010/077816 |
| Event B16 | herbicide tolerance | ATCC 203059 | US-A 2003-126634 |
| Event Bt10 | insect control - herbicide | not deposited | US95-195-01p |
| | tolerance | | |
| Event Bt11 | insect control - herbicide | not deposited - commercially | US95-195-01p |
| | tolerance | available Agrisure ™ CB/LL | |
| Event Bt176 | insect control - herbicide | not deposited - commerically | US94-319-01p |
| | tolerance | available NaturGard KnockOut ™, | |
| | | Maximizer ™ | |
| Event BVLA430101 | quality trait | not deposited | CN101792812 |
| Event CBH-351 | insect control - herbicide | not deposited - commercially | US97-265-01p |
| | tolerance | available Starlink ™ Maize | |
| Event DAS40278 | herbicide tolerance | ATCC PTA-10244 | WO2011/022469 |
| Event DAS-59122-7 | insect control - herbicide | ATCC PTA 11384 | US-A 2006-070139 |
| | tolerance | | |
| Event DAS-59132 | insect control - herbicide | not deposited - commercially | WO2009/100188 |
| | tolerance | availble Herculex ™ RW | |
| Event DBT418 | insect control - herbicide | not deposited - commercially | US96-291-01p |
| | tolerance | available Bt Xtra ™ Maize | |
| Event DP- 004114-3 | insect control | ATCC PTA-11506 | WO2011/084621 |
| Event DP-023211-2 | insect control | ATCC PTA-124722 | WO2019/209700 |
| Event DP-032316-8 | insect control | ATCC PTA-11507 | WO2011/084632 |
| Event DP-033121 | insect control - herbicide | ATCC PTA-13392 | WO2014116854 |
| | tolerance | | |
| Event DP-040416-8 | insect control | ATCC PTA-11508 | WO2011/075593 |
| Event DP-043A47-3 | insect control | ATCC PTA-11509 | WO2011/075595 |
| Event DP-098140-6 | herbicide tolerance | ATCC PTA-8296 | US-A 2009- 137395 or |
| | | | WO2008/112019 |
| Event DP-32138-1 | hybridization system | ATCC PTA-9158 | US-A 2009-0210970 or |
| | | | WO2009/103049 |
| Event Fi117 | herbicide tolerance | ATCC 209031 | US-A 2006-059581 or WO |
| | | | 98/044140 |
| Event GA21 | herbicide tolerance | ATCC 209033 | US-A 2005-086719 or WO |
| | | | 98/044140 |
| Event GG25 | herbicide tolerance | ATCC 209032 | US-A 2005-188434 or |
| | | | WO98/044140 |
| Event GJ11 | herbicide tolerance | ATCC 209030 | US-A 2005-188434 or |
| | | | WO98/044140 |
| Event HCEM485 | herbicide tolerance | ATCC PTA-12014 | WO2013/025400 |
| Event LY038 | quality trait | ATCC PTA-5623 | US-A 2007-028322 or |
| | | | WO2005/061720 |
| Event MIR162 | insect control | ATCC PTA-8166 | US-A 2009-300784 or |
| | | | WO2007/142840 |
| Event MIR604 | insect control | not deposited - commercially | US-A 2008-167456 or |
| | | available Optimum ™ Intrasect | WO2005/103301 |
| | | Xtreme | |
| Event MON801 | insect control | not deposited | US95-093-01p |
| Event MON802 | insect control | not deposited | US96-317-01p |
| Event MON809 | insect control | not deposited | US96-017-01p |
| Event MON810 | insect control | not deposited - commercially | US-A 2002-102582 |
| | | available YieldGard ™, MaizeGard ™ | |
| Event MON832 | herbicide tolerance | not deposited commercially | US96-317-01p |
| | | available Roundup Ready ™ Maize | |
| Event MON863 | insect control | ATCC PTA-2605 | WO2004/011601 or US-A |
| | | | 2006-095986 |
| Event MON87403 | quality trait | ATCC PTA- 13584 | WO2015/053998 |
| Event MON87411 | insect control - herbicide | ATCC PTA- 12669 | WO2013/169923 |
| | tolerance | | |
| Event MON87419 | herbicide tolerance | ATCC PTA- 120860 | WO2015/142571 |
| Event MON87427 | pollination control | ATCC PTA-7899 | WO2011/062904 |
| Event MON87429 | herbicide tolerance | ATCC PTA-124635 | WO2019/152316 |
| Event MON87460 | stress tolerance | ATCC PTA-8910 | WO2009/111263 or US-A |
| | | | 2011-0138504 |
| Event MON88017 | insect control - herbicide | ATCC PTA-5582 | US-A 2008-028482 or |
| | tolerance | | WO2005/059103 |
| Event MON89034 | insect control | ATCC PTA-7455 | WO2007/140256 or US-A |
| | | | 2008-260932 |
| Event MON95379 | insect control | ATCC PTA-125027 | WO2020/028172 |
| Event MS3 | hybridization system | not deposited - commercially | US95-228-01p |
| | | available InVigor ™ Maize | |
| Event MS6 | hybridization system | not deposited - commercially | US95-228-01p |
| | | available InVigor ™ Maize | |
| Event MZDT09Y | quality trait | ATCC PTA-13025 | WO2013/012775 |
| Event MZHG0JG | herbicide tolerance | ATCC PTA-122835 | WO2017/214074 |
| Event MZIR098 | insect control - herbicide | ATCC PTA-124143 | WO2018/231890 |
| | tolerance | | |
| Event NK603 | herbicide tolerance | ATCC PTA-2478 | US-A 2007-292854 |
| Event PH-000676 | herbicide tolerance - | not deposited | US97-342-01p |
| | hybridization | | |
| Event PH-000678 | herbicide tolerance - | not deposited | US97-342-01p |
| | hybridization | | |
| Event PH-000680 | herbicide tolerance - | not deposited | US97-342-01p |
| | hybridization | | |
| Event T14 | herbicide tolerance | not deposited - commercially | US94-357-14p |
| | | available Liberty Link ™ Maize | |
| Event T25 | herbicide tolerance | not deposited - commercially | US-A 2001-029014 or |
| | | available Liberty Link ™ Maize | WO2001/051654 |
| Event TC1507 | insect control - herbicide | not deposited - commerically | US-A 2005-039226 or |
| | tolerance | available Herculex ™ I, Herculex ™ | WO2004/099447 |
| | | CB | |
| Event TC6275 | insect control - herbicide | not deposited | US03-181-01p |
| | tolerance | | |
| Event VCO-01981-5 | herbicide tolerance | NCIMB 41842 | US-A 20180251778 or |
| | | | WO2013/014241 |
| Event VIP1034 | insect control - herbicide | ATCC PTA-3925 | WO2003/052073 |
| | tolerance | | |

Examples of transgenic or other events providing an additional beneficial trait may also include any of the transgenic events provided by the United States Department of Agriculture's (USDA) Animal and Plant Health Inspection Service (APHIS), which can be found at aphis.usda.gov, and/or the ISAAA (International Service for the Acquisition of Agri-Biotech Applications, which can be found at www.isaaa.org/gmapprovaldatabase.

The aforementioned additional beneficial trait(s) may be introduced into a modified corn plant or plant part by crossing or breeding a modified or transgenic corn plant comprising a mutant allele of the brachytic or brachytic2 (br2) gene or locus with a transgenic corn plant comprising the transgenic event that conveys the additional beneficial trait(s) of interest, and selecting progeny plants comprising both the mutant allele of the brachytic or brachytic2 (br2) gene or locus and the additional transgenic event conveying the additional beneficial trait. Such progeny can be identified with or without the help of molecular markers. The aforementioned additional beneficial trait(s) may also be introduced into a modified corn plant or plant part comprising a mutant allele of the brachytic or brachytic2 (br2) gene or locus using any suitable transformation, genome editing or molecular technology or technique known in the art, including but not limited to, any particle bombardment, bacteria-mediated or Agrobacterium-mediated transformation, or other known plant transformation technique, Targeting Induced Local Lesions in Genomes (TILLING), and genome editing tool, such as a zinc-finger nucleases (ZFN), transcription activator-like effector nucleases (TALEN), meganucleases and CRISPR associated systems with Cas9, Cpf1 or other site-specific nuclease. Alternatively, a mutant allele of the brachytic or brachytic2 (br2) gene or locus can be made by

Having described the present disclosure in detail, it will be apparent that modifications, variations, and equivalent aspects are possible without departing from the spirit and scope of the present disclosure as described herein and in the appended claims. Furthermore, it should be appreciated that all examples in the present disclosure are provided as non-limiting examples.

### EXAMPLES

### Example 1. Creating Dominant Alleles by Genome Editing to Produce Inverted or Hairpin-Containing Transcripts

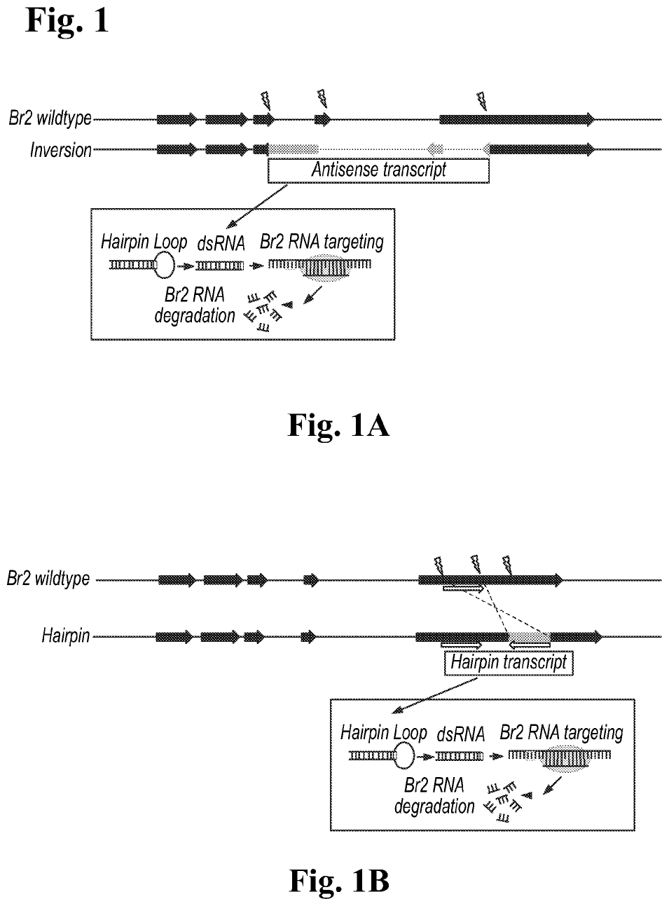

FIG. 1 provides illustrative examples for the production, through targeted genome editing, of a genetic modification of the Zm·Br2 locus (SEQ ID NO. 1), to encode a RNA transcript with an inverted sequence that may hybridize to a corresponding sense sequence of another RNA transcript in a heterozygous plant (FIG. 1A), or an inverted sequence that can hybridize to a corresponding sequence of the same RNA transcript to produce a hairpin or stem-loop structure (FIG. 1B), to cause the suppression of one or both of the copies or alleles at the endogenous Zm·Br2 locus. The insertion site is determined by the design of the guide RNA(s) directing one or more double-stranded genomic DNA cleavage(s). Without being bound to any particular theory, the edited Zm·Br2 allele may produce a RNA transcript having an antisense sequence, or able to form a stem-loop structure, which can induce suppression or gene silencing of the wild-type or other allele(s) of the Zm·Br2 gene. The inverted Zm·Br2 fragment or insertion sequence can be excised from either copy or allele of the endogenous Zm·Br2 gene. For example, an inverted Zm·Br2 fragment or insertion sequence can be excised from either copy or allele of the endogenous Zm·Br2 gene and inserted into another copy or allele of the Zm·Br2 gene. This type of editing can be referred to as a “homologous-fragment targeting” or HFT method. As another example, an inverted Zm·Br2 fragment or insertion sequence can be excised from a copy or allele of the endogenous Zm·Br2 gene and inserted into same copy or allele of the Zm·Br2 gene. This type of editing can be referred to as a “cis-fragment targeting” method. According to another approach, the inserted Zm·Br2 fragment or insertion sequence can also be excised from a DNA donor template comprising the desired Zm·Br2 fragment or sequence and flanked by target sites for two guide RNAs, which can be referred to as a “template assist” method when performed in combination with guide RNAs targeted for inserting the excised fragment or sequence from the donor template into the endogenous Zm·Br2 locus. In a further aspect, an inserted DNA fragment or sequence can be excised from another chromosomal location, which can be referred to as a “trans-fragment template” or TFT method). Regardless of the approach, the boundaries of the excision fragments or insertion sequences can be defined by two or more properly designed guide RNAs.

Two plant transformation constructs were designed to create double stranded breaks (DSB) in the Zm·Br2 gene to allow for excision and insertion of an antisense DNA fragment or sequence into the Zm·Br2 gene. In this example, the constructs generally contain 2 functional regions or cassettes relevant to gene editing and creation of the insertion (e.g., inversion) in the edited gene: expression of a Cpf1 or Cas12a variant protein, and expression of three guide RNAs for the Zm·Br2 gene locus (see, e.g., the two alternative expression cassettes below). Each guide RNA unit contains a common scaffold compatible with the Cpf1 mutant, and a unique spacer/targeting sequence complementary to its intended target site. The Cpf1 expression cassette comprises a maize ubiquitin promoter (SEQ ID NO: 39) operably linked to a sequence encoding a Lachnospiraceae bacterium G532R/K595R mutant Cpf1 RNA-guided endonuclease enzyme (SEQ ID NO: 40) fused to a nuclear localization signal at both the 5′ and 3′ ends of the transcript (SEQ ID NO: 41). See, e.g., Gao, L. et al., Nature Biotechnol. 35(8): 789-792 (2017), the entire contents and disclosure of which are incorporated herein by reference.

One expression cassette comprises a sequence encoding three guide RNAs (sequences encoded by the SP1, SP2, and SP3 DNA sequences in Table 2 below (see also FIG. 1) that target three sites in exons 3, 4 and 5 of the Zm·Br2 gene, respectively), operably linked to a maize RNA polymerase III (Pol3) promoter (SEQ ID NO: 42). Another expression cassette comprises a sequence encoding three guide RNAs (sequences encoded by the SP4, SP5, and SP6 DNA sequences in Table 2 below (see also FIG. 1) that target three sites in exon 5 of the Zm·Br2 gene, operably linked to a maize RNA polymerase III (Pol3) promoter (SEQ ID NO: 42).

With the constructs described in this example, as shown in FIG. 1A, guide RNAs with spacers SP1 and SP2 may work in combination with SP3, or SP2 and SP3 may work in combination with SPI to produce a fragment between about 860 bp and 2.4 kb from exons 3 to 5 of the endogenous Zm·Br2 gene that could be inserted into a site within exon 3 to 5 of the endogenous Zm·Br2 gene in the reverse complementary orientation, depending on any deletions and the cutting and insertion of the inversion sequence, such that the RNA molecule transcribed from the edited Zm·Br2 gene comprises an antisense sequence complementary to a corresponding sequence of the br2 locus or gene. The presence of an antisense sequence in the RNA transcript expressed from the edited br2 allele comprising the inversion or insertion sequence may trigger suppression or silencing of the other allele(s) of the endogenous Zm·Br2 gene. In addition, as shown in FIG. 1B, guide RNAs with spacers SP4 and SP5 may work in combination with SP6, or SP5 and SP6 may work in combination with SP4 to produce a fragment between about 200 bp and 450 bp from exon 5 of the endogenous Zm·Br2 gene that could be inserted into a site within exon 5 of the endogenous Zm·Br2 gene in the reverse complementary orientation, depending on any deletions and the cutting and insertion of the inversion sequence, such that the RNA molecule transcribed from the edited Zm·Br2 gene comprises an antisense sequence complementary to a neighboring corresponding sequence in the RNA molecule that may form a hairpin or stem-loop structure in the RNA transcript. Such hairpin or stem-loop structure in the RNA transcript may trigger suppression or silencing of the other allele(s) of the endogenous Zm·Br2 gene.

The DNA sequences encoding the guide RNA spacers and their intended target sites are listed in Table 2.

TABLE 2
Example guide RNAs used for editing the Zm.Br2
locus.

| Guide | | |
|---|---|---|
| RNA | | SEQ |
| Spacer | Spacer Sequence | ID |
| SP1 | GCTCATCGAGAGGTTCTACGACC | 2 |
| SP2 | ATGATGAAGGAGTGGGCGTTGGC | 3 |
| SP3 | CGATCTCGCGCTTCATGTACCGC | 4 |
| SP4 | GTGGGCAGAAGCAGCGCATCGCC | 5 |
| SP5 | GCCGCCTCTCCGACTTCTCCACC | 6 |
| SP6 | CCTACATCCTCAGCGCCGTGCTC | 7 |

### Example 2. Creation and Identification of Edits at R0 Generation

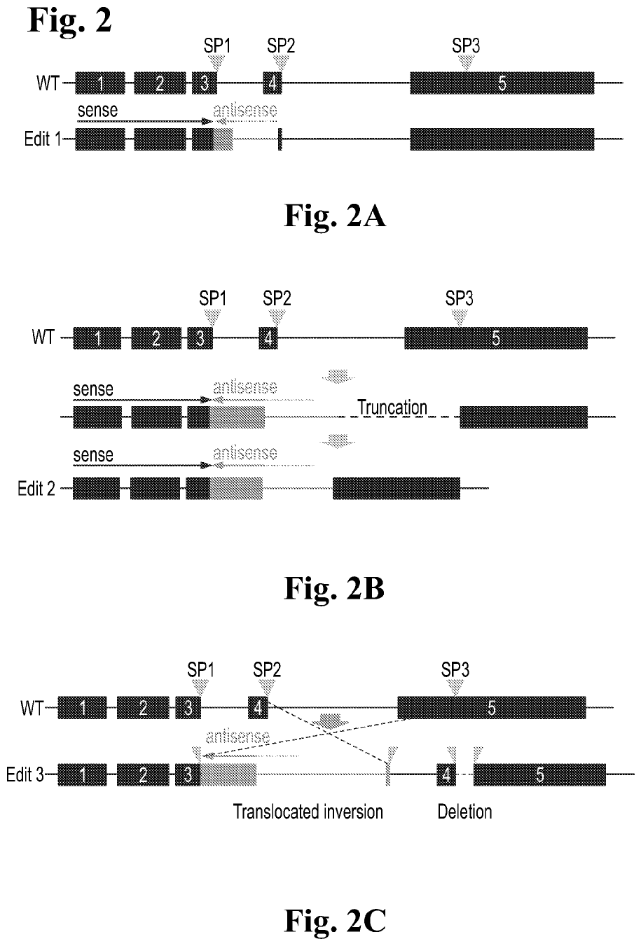

An inbred corn plant line was transformed via Agrobacterium-mediated transformation with one of the transformation vectors described above in Example 1. The transformed plant tissues were grown to mature R0 plants. R0 plants were outcrossed to wildtype corn plants of the same inbred to produce F1 inbred plants. To determine the edits and insertions in the endogenous Zm·Br2 gene of the R0 and F1 plants, a PCR assay was performed, with primers designed to identify the size or junctions of the intended insertions. One approach to identify inversions and insertions between spacers SP1, SP2, and SP3 used a PCR primer pair including one primer (SEQ ID NO: 8) hybridizing to a sequence upstream of SP1, and another primer (SEQ ID NO: 9) hybridizing to a sequence downstream of SP3. One approach to identify inversions and insertions between spacers SP4, SP5, and SP6 used a PCR primer pair including one primer (SEQ ID NO: 10) hybridizing to a sequence upstream of SP4, and another primer (SEQ ID NO: 9) hybridizing to a sequence downstream of SP6. Thus, the presence and size of the PCR fragments using these approaches would show whether an insertion occurred at the target sites, but independent of orientation. The PCR product can also be sequenced to determine the type and orientation of the insertion. Based on these sequencing results, gene models of the edited sequences were created. All edits are described in TABLE 3, and edits 1, 2, and 3 are illustrated FIGS. 2A, 2B and 2C, respectively. R0 plants were outcrossed to wildtype plants of the same inbred. Resulting F1 plants were assayed in a similar fashion (PCR and sequencing) for the type of insertion and the zygosity of the insertion mutant or allele (see TABLE 4).

TABLE 3
Inversion edits identified at R0, individual alleles confirmed by sequencing at F1.

| | | | SEQ ID | SEQ ID | SEQ ID | SEQ ID | SEQ ID |
|---|---|---|---|---|---|---|---|
| R0 | | | of 5′ | of 3′ | of | of | of |
| plant | | Edit | inversion | inversion | mutation | mutation | mutation |
| ID | EditID | description | junction | junction | at SP1 | at SP2 | at SP3 |
| 1 | 1 | Inversion | 11 | 12 | INV | INV | NONE |
| | | between guide | | | | | |
| | | SP1 and SP2 | | | | | |
| 1 | 4 | Simple deletions | NA | NA | 22 | 23 | 24 |
| | | at SP1, SP2, SP3 | | | | | |
| 2 | 2 | Inversion | 13 | 14 | INV | INV | INV |
| | | between guide | | | | | |
| | | SP1 and SP3, | | | | | |
| | | with truncation | | | | | |
| | | at 3′ end of | | | | | |
| | | inversion | | | | | |
| 3 | 3 | Inversion | 15 | 16 | INV | INV | INV |
| | | between guide | | | | | |
| | | SP2 and SP3, | | | | | |
| | | inserted into cut | | | | | |
| | | site of SP1 | | | | | |
| 4 | 5 | Inversion | 17 | 18 | INV | INV | 21 |
| | | between guide | | | | | |
| | | SP1 and SP2 | | | | | |
| 4 | 6 | Simple deletions | NA | NA | 25 | 26 | 27 |
| | | at SP1, SP2, SP3 | | | | | |
| 5 | 7 | Inversion | 19 | 20 | INV | INV | NONE |
| | | between guide | | | | | |
| | | SP1 and SP2 | | | | | |
| 5 | 8 | Simple deletions | NA | NA | 28 | 29 | NONE |
| | | at SP1 and SP2 | | | | | |
| 6 | 9 | Inversion | 30 | 31 | INV | INV | NONE |
| | | between guide | | | | | |
| | | SP2 and SP3, | | | | | |
| | | inserted into cut | | | | | |
| | | site of SP1 to | | | | | |
| | | SP3 | | | | | |
| 6 | 10 | Deletion | NA | NA | 32 | | 33 |
| | | between SP1 and | | | | | |
| | | SP2; simple | | | | | |
| | | deletion at SP3 | | | | | |
| 7 | 11 | Inversion | 34 | 35 | INV | INV | NONE |
| | | between guide | | | | | |
| | | SP1 and SP2 | | | | | |
| 7 | 12 | Simple deletions | NA | NA | 36 | 37 | 38 |
| | | at SP1, SP2, SP3 | | | | | |

### Example 3. Description of the F1 Plants and Related Data

As mentioned in Example 2, R0 plants were outcrossed to wildtype corn plants of the same inbred to produce F1 plants. F1 plants were first screened by PCR to identify nuclease-null and inversion-positive plants. Segregation of the inversion edits in F1 progeny appeared to segregate normally. Inversion-positive F1 plants were then confirmed by sequencing to be heterozygous for edits, using sequencing primers as described in example 2. F1 plants that were transplanted and kept to maturity are summarized in TABLE 4. All plants in TABLE 4 are nuclease-null, so the edit should be fixed in this and subsequent generations inheriting the edit. Edited F1 plants were phenotyped for plant height (PHT) at 7 weeks afer planting, and one week before tasseling stage (VT), which is also summarized in TABLE 4. F1 plants were grown in two batches in separate greenhouses, which is noted in the first column of TABLE 4, and statistical comparisons are made to the wildtype plants grown in the same greenhouse (see TABLES 5 and 6). Four out of five wildtype plants in GH1 were detasseled prior to maturity, which damaged the top of the plant and prevented accurate PHT measurement at VT stage, and thus statistics were not run on the “PHT at VT” column. In this experiment, none of the edits in GH2 had a significant reduction in PHT at 7 weeks after planting, and no further height measurements were taken.

TABLE 4
Nuclease-negative, edit positive F1 plants kept to maturity.

| | F1 | | PHT 7 | PHT | |
|---|---|---|---|---|---|
| | plant | Edit | weeks after | one week | PHT |
| GH | ID | ID | planting | before VT | at VT |
| 1 | 8 | 4 | 21 | 48 | 62 |
| 1 | 9 | 4 | 23 | 43 | 60 |
| 1 | 10 | 4 | 21 | 47 | 65 |
| 1 | 11 | 4 | 21.5 | 44 | 61 |
| 1 | 12 | 4 | 21.5 | 48 | 66 |
| 1 | 13 | 4 | 19 | 46 | 63 |
| 1 | 14 | 1 | 21 | 45 | 64 |
| 1 | 15 | 1 | 24 | 46 | 64 |
| 1 | 16 | 1 | 22.5 | 44 | 62 |
| 1 | 17 | 1 | 26 | 50 | 68 |
| 1 | 18 | 1 | 21 | 42 | 61 |
| 1 | 19 | 1 | 24 | 53 | 67 |
| 1 | 20 | 1 | 20.5 | 43 | 66 |
| 1 | 21 | 1 | 21 | 48 | 64 |
| 1 | 22 | 1 | 21 | 48 | 61 |
| 1 | 23 | 1 | 22 | 46 | 58 |
| 1 | 24 | 1 | 24 | 44 | 66 |
| 1 | 25 | 1 | 23 | 43 | 62 |
| 1 | 26 | 1 | 24 | 49 | 62 |
| 1 | 27 | 1 | 24 | 50 | 72 |
| 1 | 28 | 1 | 22 | 46 | 69 |
| 1 | 29 | 1 | 21 | 40 | 60 |
| 1 | 30 | 1 | 23 | 45 | 63 |
| 1 | 31 | 3 | 22.5 | 53 | 68 |
| 1 | 32 | 3 | 22.5 | 49 | 59 |
| 1 | 33 | 3 | 22.5 | 51 | 63 |
| 1 | 34 | 2 | 26.5 | 46 | 59 |
| 1 | 35 | 2 | 25 | 49 | 64 |
| 1 | 36 | 2 | 24.5 | 53 | 67 |
| 1 | 37 | 2 | 22 | 45 | 62 |
| 1 | 38 | 2 | 23 | 48 | 63 |
| 1 | 39 | 2 | 23 | 49 | 63 |
| 1 | 40 | 10 | 26 | 52 | 68 |
| 1 | 41 | 10 | 26 | 63 | 69 |
| 1 | 42 | 9 | 25.5 | 58 | 69 |
| 1 | 43 | 9 | 23.5 | 56 | 65 |
| 1 | 44 | 9 | 27.5 | 59 | 61 |
| 1 | 45 | 9 | 25 | 60 | 74 |
| 1 | 46 | 9 | 30 | 64 | 76 |
| 1 | 47 | 9 | 29 | 60 | 73 |
| 1 | 48 | 9 | 32 | 56 | 68 |
| 1 | 49 | WT - GH1 | 31 | 60 | Damaged |
| 1 | 50 | WT - GH1 | 28.5 | 66 | 88 |
| 1 | 51 | WT - GH1 | 31 | 61 | Damaged |
| 1 | 52 | WT - GH1 | 28.5 | 58 | Damaged |
| 1 | 53 | WT - GH1 | 28 | 56 | Damaged |
| 2 | 54 | 6 | 28 | — | — |
| 2 | 55 | 6 | 27 | — | — |
| 2 | 56 | 6 | 28 | — | — |
| 2 | 57 | 6 | 20 | — | — |
| 2 | 58 | 6 | 27 | — | — |
| 2 | 59 | 6 | 28 | — | — |
| 2 | 60 | 5 | 26 | — | — |
| 2 | 61 | 5 | 28 | — | — |
| 2 | 62 | 5 | 29 | — | — |
| 2 | 63 | 5 | 28 | — | — |
| 2 | 64 | 5 | 29 | — | — |
| 2 | 65 | 5 | 28 | — | — |
| 2 | 66 | 5 | 26 | — | — |
| 2 | 67 | 5 | 28 | — | — |
| 2 | 68 | 5 | 28 | — | — |
| 2 | 69 | 5 | 26 | — | — |
| 2 | 70 | 5 | 29 | — | — |
| 2 | 71 | 5 | 25 | — | — |
| 2 | 72 | 5 | 26 | — | — |
| 2 | 73 | 5 | 27 | — | — |
| 2 | 74 | 5 | 29 | — | — |
| 2 | 75 | 5 | 27 | — | — |
| 2 | 76 | 5 | 29 | — | — |
| 2 | 77 | 5 | 30 | — | — |
| 2 | 78 | 5 | 30 | — | — |
| 2 | 79 | 5 | 30 | — | — |
| 2 | 80 | 5 | 29 | — | — |
| 2 | 81 | 8 | 31 | — | — |
| 2 | 82 | 8 | 29 | — | — |
| 2 | 83 | 8 | 26 | — | — |
| 2 | 84 | 7 | 27 | — | — |
| 2 | 85 | 7 | 29 | — | — |
| 2 | 86 | 7 | 29 | — | — |
| 2 | 87 | 7 | 29 | — | — |
| 2 | 88 | 7 | 26 | — | — |
| 2 | 89 | 7 | 26 | — | — |
| 2 | 90 | 7 | 27 | — | — |
| 2 | 91 | 7 | 27 | — | — |
| 2 | 92 | 12 | 29 | — | — |
| 2 | 93 | 12 | 28 | — | — |
| 2 | 94 | 11 | 28 | — | — |
| 2 | 95 | 11 | 29 | — | — |
| 2 | 96 | WT - GH2 | 30 | — | — |
| 2 | 97 | WT - GH2 | 27 | — | — |
| 2 | 98 | WT - GH2 | 28 | — | — |
| 2 | 99 | WT - GH2 | 27 | — | — |
| 2 | 100 | WT - GH2 | 25 | — | — |

TABLE 5
Nuclease-negative, edit positive F1 plants kept to maturity;
statistics on measurements taken at 7 weeks after planting
(negative percent change = plant height reduction).

| | | Avg PHT | | | | | |
|---|---|---|---|---|---|---|---|
| | | 7 weeks | Percent | | | t-statistic | p-value |
| | GH | after | change | | | compared | compared |
| Edit ID | location | planting | from WT | STDEV | N | to WT | to WT |
| 1 | 1 | 22.6 | −23% | 1.57 | 17 | 8.62 | <0.0001 |
| 2 | 1 | 24.0 | −18% | 1.64 | 6 | 5.69 | 0.0003 |
| 3 | 1 | 22.5 | −23% | 0.00 | 3 | 7.87 | 0.0002 |
| 4 | 1 | 21.2 | −28% | 1.29 | 6 | 9.86 | <0.0001 |
| 5 | 2 | 28.0 | 2% | 1.50 | 21 | 0.77 | 0.4465 |
| 6 | 2 | 26.3 | −4% | 3.14 | 6 | 0.69 | 0.5081 |
| 7 | 2 | 27.5 | 0% | 1.31 | 8 | 0.12 | 0.9099 |
| 8 | 2 | 28.7 | 5% | 2.52 | 3 | 0.86 | 0.4249 |
| 9 | 1 | 27.5 | −6% | 3.03 | 7 | 1.29 | 0.2276 |
| 10 | 1 | 26.0 | −12% | 0.00 | 2 | 3.09 | 0.0271 |
| 11 | 2 | 28.5 | 4% | 0.71 | 2 | 0.79 | 0.4639 |
| 12 | 2 | 28.5 | 4% | 0.71 | 2 | 0.79 | 0.4639 |
| WT - GH1 | 1 | 29.4 | 0% | 1.47 | 5 | — | — |
| WT - GH2 | 2 | 27.4 | 0% | 1.82 | 5 | — | — |

TABLE 6
Nuclease-negative, edit positive F1 plants kept to maturity; statistics on measurements
taken at 1 week before VT (negative percent change = plant height reduction).

| | | Avg PHT 1 | Percent | | | t-statistic | p-value |
|---|---|---|---|---|---|---|---|
| | GH | week before | change from | | | compared | compared |
| Edit ID | location | VT | WT | STDEV | N | to WT | to WT |
| 1 | 1 | 46.0 | −24% | 3.34 | 17 | −8.14 | <0.0001 |
| 2 | 1 | 48.3 | −20% | 2.80 | 6 | −6.02 | 0.0002 |
| 3 | 1 | 51.0 | −15% | 2.00 | 3 | −3.83 | 0.0086 |
| 4 | 1 | 46.0 | −24% | 2.10 | 6 | −7.92 | <0.0001 |
| 5 | 2 | — | — | — | — | — | — |
| 6 | 2 | — | — | — | — | — | — |
| 7 | 2 | — | — | — | — | — | — |
| 8 | 2 | — | — | — | — | — | — |
| 9 | 1 | 59.0 | −2% | 2.77 | 7 | −0.64 | 0.5372 |
| 10 | 1 | 57.5 | −4% | 7.78 | 2 | −0.67 | 0.5349 |
| 11 | 2 | — | — | — | — | — | — |
| 12 | 2 | — | — | — | — | — | — |
| WT - GH1 | 1 | 60.2 | 0% | 3.77 | 5 | — | — |
| WT - GH2 | 2 | — | — | — | — | — | — |

### Example 4. Phenotypes and RNA Expression in F2 Plants

F1 plants were kept to maturity and self-pollinated to produce F2 progeny. F2 plants were sequenced using primers and method as in Example 2. To determine edit zygosity, the ratio of WT to NV (inversion) reads for each F2 population was plotted on a scatterplot, and homozygous, heterozygous, and null segregants were easily determined. Because F1 plants were negative for the Cpf1 nuclease and heterozygous for the inversion edits, the inversion edits are expected to segregate in a 1:2:1 Mendelian fashion at F2. Segregation of F2 generation is summarized in TABLE 7.

TABLE 7
segregation of inversion edits at F2 generation

| Edit | Edit zygosity | | | |
|---|---|---|---|---|
| ID | NULL | HET | HOMO | Total |
| 1 | 32 | 65 | 30 | 127 |
| 2 | 22 | 57 | 30 | 109 |
| 3 | 31 | 48 | 24 | 103 |

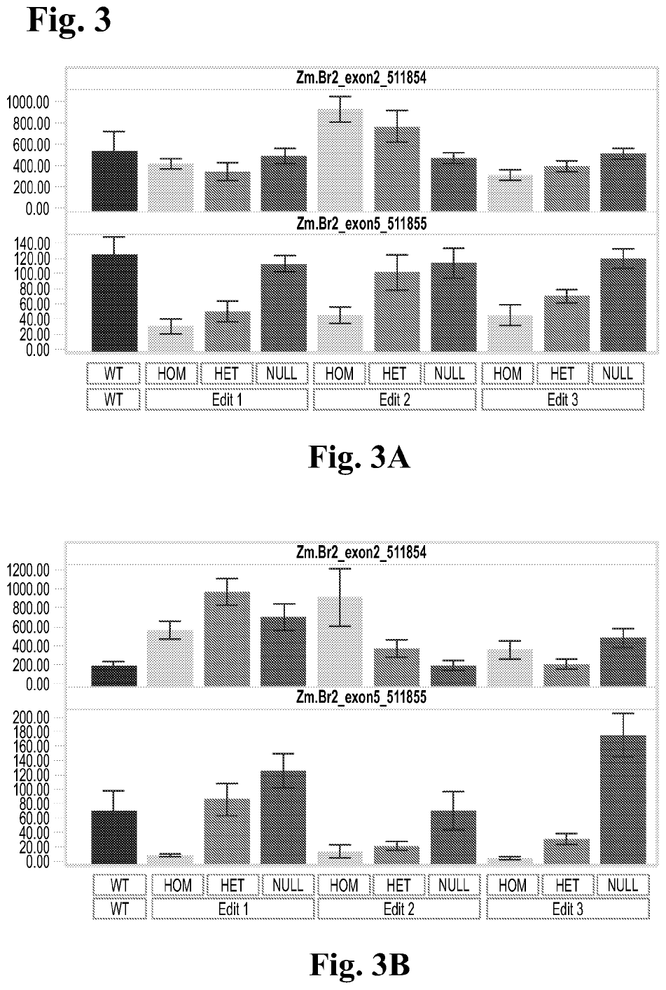

Eight plants for each edit by zygosity combination were sampled at the third leaf base for RNA expression analysis. Two RNA assays were designed: one for exon 2, and one for exon 5. Assay primers and probes are described in TABLE 8. Total RNA was extracted from the samples, and data was normalized to the Zm·EF1a gene. Results are summarized in FIG. 3A and TABLES 9 and 10. Homozygous and heterozygous edited plants had reduced Zm·Br2 mRNA expression compared to wildtype and null-segregated siblings for edits 1 and 3. For edit 2, Zm·Br2 exon2 expression was increased in homozygous and heterozygous edited plants, but exon 5 expression was decreased.

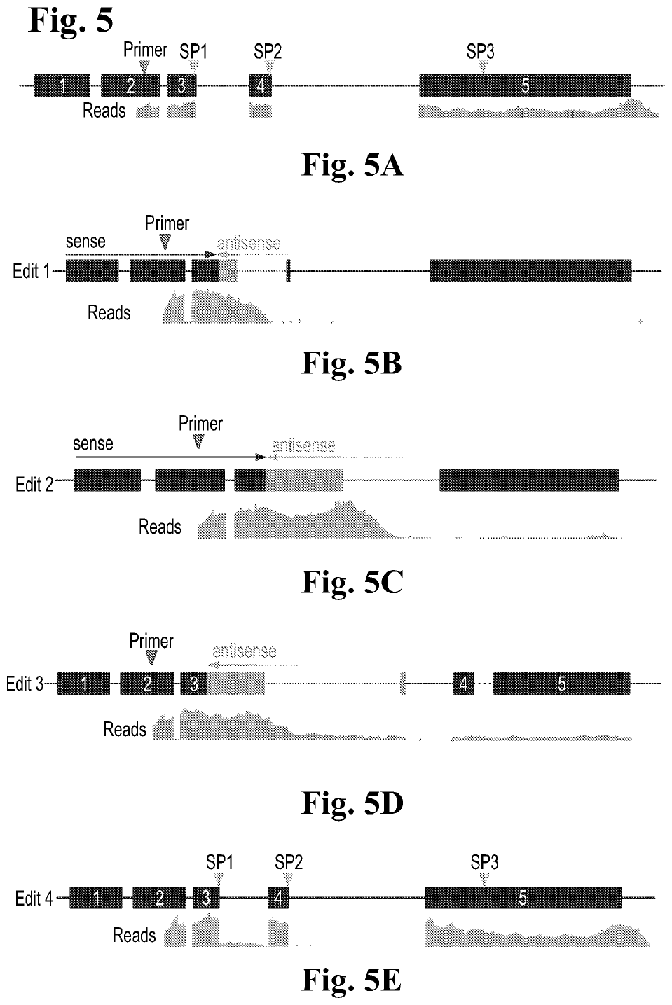

It has been previously observed that the br2 gene locus may produce two distinct mRNA species likely through alternative splicing with the primary RNA transcript species expressed from the br2 locus being spliced according to the exons 1-5 as described herein, and the alternative mature mRNA transcript not being spliced between exons 4 and 5 as for the primary transcript, such that at least part of intron 4 becomes part of the alternative mature RNA transcript including the additional “GTCCGTCCCGTATAG” sequence with the “TAG” providing a stop codon at the 3′ end of exon 4. See, e.g., Zhang, X. et al., BMC Plant Biology 19:589 (2019), the contents and disclosure of which are incorporated herein by reference. As a result, the alternative mature RNA transcript will lack exon 5 of the br2 locus. Therefore, RNA expression analysis using an oligonucleotide probe for exon 2 of the br2 locus would detect both RNA species, whereas a probe for exon 5 would only detect the primary transcript. In addition, RNA transcripts expressed from edited alleles of the br2 locus may be altered or truncated prior to exon 5 for transcriptional and/or post-transcriptional reasons, such that these edited allele transcripts are not detected by the probe for exon 5 of the br2 locus (see, e.g. FIG. 5 described below). Without being bound by theory, it is hypothesized that a truncated Br2 protein encoded by the alternatively spliced RNA transcript or RNA transcripts expressed from the edited alleles of the br2 locus may be non-functional or have reduced function relative to the wild-type Br2 protein encoded by the primary RNA transcript. In addition, RNA transcripts expressed from the edited alleles of the br2 locus may also lead to suppression or silencing of, and/or degradation or decay of the RNA transcript expressed from, the other copy or allele of the br2 locus or gene.

TABLE 8
RNA assay primer and probe designs.

| | | Primer or | |
|---|---|---|---|
| SEQ ID | Exon | probe | Sequence |
| 43 | 2 | Primer_FWD | CAGATACGGATCGTGCAGG |
| 44 | 2 | Primer_RVS | GCACCTACTTCACCGTCTTC |
| 47 | 2 | Probe | TGGCGCAGAGGATCGGCTAC |
| 45 | 5 | Primer_FWD | TGGACTTCTCGTACCCGTC |
| 46 | 5 | Primer_RVS | CTGGTGCAGCGGTTCTAC |
| 48 | 5 | Probe | TTCCGCGACCTGAGCCTCC |

TABLE 9
RNA expression analysis at third leaf base, exon 2 assay.

| | | | | | t-statistic | p-value |
|---|---|---|---|---|---|---|
| | | Relative | | | compared | compared |
| Edit | Zygosity | expression | STDEV | N | to WT | to WT |
| 1 | HOM | 444.5 | 148.2 | 11 | −0.74 | 0.4683 |
| 1 | HET | 378.0 | 233.3 | 8 | −0.94 | 0.3612 |
| 1 | NULL | 518.0 | 181.4 | 8 | −0.23 | 0.8209 |
| 2 | HOM | 944.0 | 379.2 | 11 | 1.91 | 0.0731 |
| 2 | HET | 784.8 | 409.1 | 8 | 0.98 | 0.3421 |
| 2 | NULL | 501.0 | 117.3 | 8 | −0.33 | 0.7439 |
| 3 | HOM | 343.8 | 163.5 | 11 | −1.37 | 0.1898 |
| 3 | HET | 420.2 | 142.7 | 8 | −0.77 | 0.4533 |
| 3 | NULL | 535.1 | 131.5 | 8 | −0.14 | 0.8882 |
| WT | WT | 561.1 | 496.5 | 8 | — | — |

TABLE 10
RNA expression analysis at third leaf base, exon 5 assay.

| | | | | | t-statistic | p-value |
|---|---|---|---|---|---|---|
| | | Relative | | | compared | compared |
| Edit | Zygosity | expression | STDEV | N | to WT | to WT |
| 1 | HOM | 33.7 | 31.3 | 11 | −4.27 | 0.0005 |
| 1 | HET | 53.3 | 38.7 | 8 | −2.82 | 0.0137 |
| 1 | NULL | 119.9 | 30.2 | 8 | −0.28 | 0.7807 |
| 2 | HOM | 48.1 | 33.8 | 11 | −3.54 | 0.0025 |
| 2 | HET | 103.8 | 63.3 | 8 | −0.73 | 0.4761 |
| 2 | NULL | 115.7 | 55.5 | 8 | −0.38 | 0.7114 |
| 3 | HOM | 48.0 | 45.1 | 11 | −3.19 | 0.0053 |
| 3 | HET | 73.7 | 25.0 | 8 | −2.22 | 0.0432 |
| 3 | NULL | 120.7 | 32.6 | 8 | −0.25 | 0.8081 |
| WT | WT | 126.9 | 62.9 | 8 | — | — |

Twenty-seven F2 plants per edit were transplanted. For each HOMO/HET/NULL set of F2 plants or inventory for each edit, 9 plants were kept to maturity: 5 plants homozygous for the inversion edit, 2 plants heterozygous for the inversion edit, and 2 plants null for the inversion edit. In addition, for each F2 inventory, 18 plants (6 from each zygosity) were transplanted and intended to be destructively sampled for RNA assay at V10 stage. See TABLE 11 for description of each transplanted F2 plant.

TABLE 11
F2 inversion, deletion and wildtype inbred plants transplanted.

| | | | | | Plant height | Plant height |
|---|---|---|---|---|---|---|
| Plant | Edit | Inbred | | Maturity or | 42 days after | 56 days after |
| ID | ID | ID | Zygosity | sample/discard | planting | planting |
| 101 | 1 | 1 | HOMO | maturity | 13 | 32 |
| 102 | 1 | 1 | HOMO | maturity | 12 | 32 |
| 103 | 1 | 1 | HOMO | maturity | 12 | 35 |
| 104 | 1 | 1 | HOMO | maturity | 13 | 31 |
| 105 | 1 | 1 | HOMO | maturity | 13 | 32 |
| 106 | 1 | 1 | HET | maturity | 31 | 64 |
| 107 | 1 | 1 | HET | maturity | 35 | 67 |
| 108 | 1 | 1 | NULL | maturity | 34 | 69 |
| 109 | 1 | 1 | NULL | maturity | 36 | 72 |
| 110 | 1 | 1 | HOMO | sample/discard at V10 | | |
| 111 | 1 | 1 | HOMO | sample/discard at V10 | | |
| 112 | 1 | 1 | HOMO | sample/discard at V10 | | |
| 113 | 1 | 1 | HOMO | sample/discard at V10 | | |
| 114 | 1 | 1 | HOMO | sample/discard at V10 | | |
| 115 | 1 | 1 | HOMO | sample/discard at V10 | | |
| 116 | 1 | 1 | HET | sample/discard at V10 | | |
| 117 | 1 | 1 | HET | sample/discard at V10 | | |
| 118 | 1 | 1 | HET | sample/discard at V10 | | |
| 119 | 1 | 1 | HET | sample/discard at V10 | | |
| 120 | 1 | 1 | HET | sample/discard at V10 | | |
| 121 | 1 | 1 | HET | sample/discard at V10 | | |
| 122 | 1 | 1 | NULL | sample/discard at V10 | | |
| 123 | 1 | 1 | NULL | sample/discard at V10 | | |
| 124 | 1 | 1 | NULL | sample/discard at V10 | | |
| 125 | 1 | 1 | NULL | sample/discard at V10 | | |
| 126 | 1 | 1 | NULL | sample/discard at V10 | | |
| 127 | 1 | 1 | NULL | sample/discard at V10 | | |
| 128 | 2 | 1 | HOMO | maturity | 14 | 34 |
| 129 | 2 | 1 | HOMO | maturity | 13 | 29 |
| 130 | 2 | 1 | HOMO | maturity | 13 | 30 |
| 131 | 2 | 1 | HOMO | maturity | 13 | 27 |
| 132 | 2 | 1 | HOMO | maturity | 12 | 28 |
| 133 | 2 | 1 | HET | maturity | 34 | 66 |
| 134 | 2 | 1 | HET | maturity | 32 | 57 |
| 135 | 2 | 1 | NULL | maturity | 34 | 59 |
| 136 | 2 | 1 | NULL | maturity | 35 | 62 |
| 137 | 2 | 1 | HOMO | sample/discard at V10 | | |
| 138 | 2 | 1 | HOMO | sample/discard at V10 | | |
| 139 | 2 | 1 | HOMO | sample/discard at V10 | | |
| 140 | 2 | 1 | HOMO | sample/discard at V10 | | |
| 141 | 2 | 1 | HOMO | sample/discard at V10 | | |
| 142 | 2 | 1 | HOMO | sample/discard at V10 | | |
| 143 | 2 | 1 | HET | sample/discard at V10 | | |
| 144 | 2 | 1 | HET | sample/discard at V10 | | |
| 145 | 2 | 1 | HET | sample/discard at V10 | | |
| 146 | 2 | 1 | HET | sample/discard at V10 | | |
| 147 | 2 | 1 | HET | sample/discard at V10 | | |
| 148 | 2 | 1 | HET | sample/discard at V10 | | |
| 149 | 2 | 1 | NULL | sample/discard at V10 | | |
| 150 | 2 | 1 | NULL | sample/discard at V10 | | |
| 151 | 2 | 1 | NULL | sample/discard at V10 | | |
| 152 | 2 | 1 | NULL | sample/discard at V10 | | |
| 153 | 2 | 1 | NULL | sample/discard at V10 | | |
| 154 | 2 | 1 | NULL | sample/discard at V10 | | |
| 155 | 3 | 1 | HOMO | maturity | 11 | 32 |
| 156 | 3 | 1 | HOMO | maturity | 12 | 37 |
| 157 | 3 | 1 | HOMO | maturity | 12 | 35 |
| 158 | 3 | 1 | HOMO | maturity | 12 | 32 |
| 159 | 3 | 1 | HOMO | maturity | 12 | 34 |
| 160 | 3 | 1 | HET | maturity | 30 | 61 |
| 161 | 3 | 1 | HET | maturity | 33 | 76 |
| 162 | 3 | 1 | NULL | maturity | 36 | 80 |
| 163 | 3 | 1 | NULL | maturity | 36 | 76 |
| 164 | 3 | 1 | HOMO | sample/discard at V10 | | |
| 165 | 3 | 1 | HOMO | sample/discard at V10 | | |
| 166 | 3 | 1 | HOMO | sample/discard at V10 | | |
| 167 | 3 | 1 | HOMO | sample/discard at V10 | | |
| 168 | 3 | 1 | HOMO | sample/discard at V10 | | |
| 169 | 3 | 1 | HOMO | sample/discard at V10 | | |
| 170 | 3 | 1 | HET | sample/discard at V10 | | |
| 171 | 3 | 1 | HET | sample/discard at V10 | | |
| 172 | 3 | 1 | HET | sample/discard at V10 | | |
| 173 | 3 | 1 | HET | sample/discard at V10 | | |
| 174 | 3 | 1 | HET | sample/discard at V10 | | |
| 175 | 3 | 1 | HET | sample/discard at V10 | | |
| 176 | 3 | 1 | NULL | sample/discard at V10 | | |
| 177 | 3 | 1 | NULL | sample/discard at V10 | | |
| 178 | 3 | 1 | NULL | sample/discard at V10 | | |
| 179 | 3 | 1 | NULL | sample/discard at V10 | | |
| 180 | 3 | 1 | NULL | sample/discard at V10 | | |
| 181 | 3 | 1 | NULL | sample/discard at V10 | | |
| 182 | WT | 1 | WT | maturity | 42 | 76 |
| 183 | WT | 1 | WT | maturity | 41 | 78 |
| 184 | WT | 1 | WT | maturity | 41 | 74 |
| 185 | WT | 1 | WT | maturity | 43 | 79 |
| 186 | WT | 1 | WT | maturity | | |
| 187 | WT | 1 | WT | maturity | | |
| 188 | WT | 1 | WT | maturity | | |
| 189 | WT | 1 | WT | maturity | | |
| 190 | WT | 1 | WT | maturity | | |
| 191 | WT | 1 | WI | maturity | | |
| 192 | WT | 1 | WT | maturity | | |
| 193 | WT | 1 | WT | sample/discard at V10 | | |
| 194 | WT | 1 | WT | sample/discard at V10 | | |
| 195 | WT | 1 | WT | sample/discard at V10 | | |
| 196 | WT | 1 | WT | sample/discard at V10 | | |
| 197 | WT | 1 | WT | sample/discard at V10 | | |
| 198 | WT | 1 | WT | sample/discard at V10 | | |
| 199 | WT | 1 | WT | sample/discard at V10 | | |
| 200 | WT | 1 | WT | sample/discard at V10 | | |
| 201 | WT | 2 | WT | maturity | | |
| 202 | WT | 2 | WT | maturity | | |
| 203 | WT | 2 | WT | maturity | | |
| 204 | WT | 2 | WT | maturity | | |
| 205 | WT | 2 | WT | maturity | | |
| 206 | WT | 2 | WT | maturity | | |
| 207 | WT | 2 | WT | maturity | | |
| 208 | WT | 2 | WT | maturity | | |
| 209 | WT | 3 | WT | maturity | | |
| 210 | WT | 3 | WT | maturity | | |
| 211 | WT | 3 | WT | maturity | | |
| 212 | WT | 3 | WT | maturity | | |
| 213 | WT | 3 | WT | maturity | | |
| 214 | WT | 3 | WT | maturity | | |
| 215 | WT | 3 | WT | maturity | | |
| 216 | WT | 3 | WT | maturity | | |

At V10 growth stage, the plants intended for RNA analysis were destructively sampled. An approximately 10 mm×3 mm section of the outer vascular section of node 6 was excised from each plant, frozen on dry ice, and total RNA was extracted. RNA assays were performed as described in Example 4. Results are summarized in FIG. 3B and TABLES 12 and 13, and expression trends are similar to those seen in samples from the 3^{rd} leaf base tissue.

TABLE 12
F2 RNA expression analysis at node 6 of V10 plants, exon 2 assay.

| | | | | | t-statistic | p-value |
|---|---|---|---|---|---|---|
| | | Relative | | | compared | compared |
| Edit | Zygosity | expression | STDEV | N | to WT | to WT |
| 1 | HOM | 584.6 | 218.1 | 6 | −3.98 | 0.0018 |
| 1 | HET | 978.1 | 327.9 | 6 | −6.05 | 0.0001 |
| 1 | NULL | 726.9 | 340.7 | 6 | −3.92 | 0.0020 |
| 2 | HOM | 928.1 | 724.6 | 6 | −2.75 | 0.0176 |
| 2 | HET | 398.7 | 218.6 | 6 | −1.95 | 0.0745 |
| 2 | NULL | 220.5 | 124.7 | 6 | −0.02 | 0.9820 |
| 3 | HOM | 384.3 | 239.6 | 6 | −1.68 | 0.1180 |
| 3 | HET | 233.4 | 113.3 | 6 | −0.22 | 0.8278 |
| 3 | NULL | 509.2 | 236.6 | 6 | −2.98 | 0.0114 |
| WT | WT | 218.95 | 125.1 | 8 | — | — |

TABLE 13
F2 RNA expression analysis at node 6 of V10 plants, exon 5 assay.

| | | | | | t-statistic | p-value |
|---|---|---|---|---|---|---|
| | | Relative | | | compared | compared |
| Edit | Zygosity | expression | STDEV | N | to WT | to WT |
| 1 | HOM | 15.1 | 5.5 | 6 | 1.793 | 0.0983 |
| 1 | HET | 90.0 | 51.8 | 6 | −0.411 | 0.6881 |
| 1 | NULL | 127.9 | 56.8 | 6 | −1.385 | 0.1912 |
| 2 | HOM | 19.8 | 17.2 | 6 | 1.627 | 0.1297 |
| 2 | HET | 27.0 | 15.4 | 6 | 1.417 | 0.1818 |
| 2 | NULL | 78.0 | 59.6 | 6 | −0.090 | 0.9301 |
| 3 | HOM | 11.0 | 5.3 | 6 | 1.917 | 0.0794 |
| 3 | HET | 36.5 | 18.5 | 6 | 1.127 | 0.2816 |
| 3 | NULL | 176.4 | 69.3 | 6 | −2.488 | 0.0286 |
| WT | WT | 74.5 | 80.2 | 8 | — | — |

Of plants kept to maturity, plant heights were measured. Individual heights are in TABLE 11 and results are summarized in TABLES 14 and 15. Plant height reductions in homozygous and heterozygous edited plants were observed for edits 1, 2, and 3 with a greater reduction in plant heights in homozygous plants.

TABLE 14
F2 plant height at 42 days after planting.

| | Average plant | | | | | Percent plant |
|---|---|---|---|---|---|---|
| | height 42 | | | | | height change |
| | days after | | | t-statistic | p-value | from WT 42 |
| | planting in | | | compared | compared | days after |
| Edit - zygosity | inches | STDEV | N | to WT | to WT | planting |
| WT | 25.38 | 0.96 | 5 | — | — | — |
| Edit 1 - HOM | 9 | 0.55 | 5 | −33.10 | <0.0001 | −65% |
| Edit 1 - HET | 21.43 | 2.83 | 2 | −3.10 | 0.0270 | −16% |
| Edit 1 - NULL | 24.25 | 1.41 | 2 | −1.27 | 0.2587 | −4% |
| Edit 2 - HOM | 9.27 | 0.71 | 5 | −30.27 | <0.0001 | −63% |
| Edit 2 - HET | 20.25 | 1.41 | 2 | −5.76 | 0.0022 | −20% |
| Edit 2 - NULL | 21.38 | 0.71 | 2 | −5.25 | 0.0033 | −16% |
| Edit 3 - HOM | 8.27 | 0.45 | 5 | −36.21 | <0.0001 | −67% |
| Edit 3 - HET | 19.06 | 2.12 | 2 | −5.91 | 0.0020 | −25% |
| Edit 3 - NULL | 20.81 | 0.00 | 2 | −6.38 | 0.0014 | −18% |

TABLE 15
F2 plant height at 56 days after planting.

| | Average plant | | | | | Percent plant |
|---|---|---|---|---|---|---|
| | height 56 days | | | t-statistic | p-value | height change |
| | after planting | | | compared | compared | from WT 56 days |
| Edit - zygosity | in inches | STDEV | N | to WT | to WT | after planting |
| WT | 76.75 | 2.22 | 5 | — | — | — |
| Edit 1 - HOM | 32.4 | 1.52 | 5 | −36.92 | <0.0001 | −58% |
| Edit 1 - HET | 65.5 | 2.12 | 2 | −6.12 | 0.0017 | −15% |
| Edit 1 - NULL | 70.5 | 2.12 | 2 | −3.40 | 0.0193 | −8% |
| Edit 2 - HOM | 59.23 | 2.70 | 5 | −11.21 | <0.0001 | −23% |
| Edit 2 - HET | 61.5 | 6.36 | 2 | −5.25 | 0.0033 | −20% |
| Edit 2 - NULL | 60.5 | 2.12 | 2 | −8.83 | 0.0003 | −21% |
| Edit 3 - HOM | 34 | 2.12 | 5 | −31.15 | <0.0001 | −56% |
| Edit 3 - HET | 68.5 | 10.61 | 2 | −1.92 | 0.1132 | −11% |
| Edit 3 - NULL | 78 | 2.83 | 2 | 0.64 | 1.4467 | +2% |

### Example 5. Description of the Hybrid Plants and Related Data

F2 homozygous edited plants of inbred 1 were crossed with plants of inbreds 2 and 3 to produce hybrid seeds. As controls, wildtype plants of inbred 1 were also crossed with plants of inbreds 2 and 3 to produce wildtype hybrid seeds. See TABLE 16 for hybrid plant information. Hybrid seeds were germinated and edit presence and zygosity was confirmed by sequencing as in above examples. Hybrid plants from edited parents were heterozygous for edits as expected.

TABLE 16
Hybrid plants

| Hybrid | Parent 1 | Parent 1 | Parent 2 | Parent 2 | Edit | Final plant | Final ear |
|---|---|---|---|---|---|---|---|
| plant ID | plant ID | inbred ID | plant ID | inbred ID | ID | height in inches | height in inches |
| 217 | 201 | 2 | 103 | 1 | WT | 110 | 55 |
| 218 | 201 | 2 | 103 | 1 | WT | 121 | 56 |
| 219 | 201 | 2 | 103 | 1 | WT | 112 | 54 |
| 220 | 201 | 2 | 103 | 1 | WT | 111 | 54 |
| 221 | 201 | 2 | 103 | 1 | WT | 112 | 46 |
| 222 | 201 | 2 | 103 | 1 | WT | 98 | 42 |
| 223 | 201 | 2 | 103 | 1 | WT | 103 | 48 |
| 224 | 201 | 2 | 103 | 1 | WT | 103 | 52 |
| 225 | 201 | 2 | 103 | 1 | WT | 107 | 54 |
| 226 | 201 | 2 | 103 | 1 | WT | 113 | 50 |
| 227 | 201 | 2 | 103 | 1 | WT | 105 | 52 |
| 228 | 201 | 2 | 103 | 1 | WT | 106 | 46 |
| 229 | 201 | 2 | 103 | 1 | WT | 103 | 53 |
| 230 | 201 | 2 | 103 | 1 | WT | 104 | 55 |
| 231 | 201 | 2 | 103 | 1 | WT | 105 | 51 |
| 232 | 201 | 2 | 103 | 1 | WT | 98 | 48 |
| 233 | 201 | 2 | 157 | 1 | WT | 126 | 62 |
| 234 | 201 | 2 | 157 | 1 | WT | 127 | 58 |
| 235 | 201 | 2 | 157 | 1 | WT | 107 | 50 |
| 236 | 201 | 2 | 157 | 1 | WT | 114 | 60 |
| 237 | 201 | 2 | 157 | 1 | WT | 105 | 47 |
| 238 | 201 | 2 | 157 | 1 | WT | 110 | 47 |
| 239 | 201 | 2 | 157 | 1 | WT | 108 | 54 |
| 240 | 103 | 2 | 157 | 1 | 1 | 100 | 47 |
| 241 | 103 | 2 | 132 | 1 | 1 | 99 | 45 |
| 242 | 103 | 2 | 132 | 1 | 1 | 99 | 46 |
| 243 | 103 | 2 | 132 | 1 | 1 | 102 | 42 |
| 244 | 103 | 2 | 132 | 1 | 1 | 111 | 47 |
| 245 | 103 | 2 | 132 | 1 | 1 | 101 | 47 |
| 246 | 103 | 2 | 209 | 1 | 1 | 98 | 53 |
| 247 | 103 | 2 | 209 | 1 | 1 | 100 | 46 |
| 248 | 103 | 2 | 209 | 1 | 1 | 101 | 48 |
| 249 | 103 | 2 | 209 | 1 | 1 | 86 | 38 |
| 250 | 103 | 2 | 209 | 1 | 1 | 101 | 48 |
| 251 | 103 | 2 | 209 | 1 | 1 | 109 | 56 |
| 252 | 103 | 2 | 209 | 1 | 1 | 96 | 48 |
| 253 | 103 | 2 | 209 | 1 | 1 | 110 | 46 |
| 254 | 103 | 2 | 209 | 1 | 1 | 107 | 50 |
| 255 | 103 | 2 | 209 | 1 | 1 | 108 | 51 |
| 256 | 157 | 2 | 209 | 1 | 3 | 94 | 47 |
| 257 | 157 | 2 | 209 | 1 | 3 | 97 | 47 |
| 258 | 157 | 2 | 209 | 1 | 3 | 100 | 48 |
| 259 | 157 | 2 | 209 | 1 | 3 | 105 | 48 |
| 260 | 157 | 2 | 209 | 1 | 3 | 98 | 46 |
| 261 | 157 | 2 | 209 | 1 | 3 | 97 | 41 |
| 262 | 157 | 2 | 209 | 1 | 3 | 105 | 52 |
| 263 | 157 | 2 | 209 | 1 | 3 | 100 | 46 |
| 264 | 132 | 2 | 209 | 1 | 2 | 95 | 45 |
| 265 | 132 | 2 | 209 | 1 | 2 | 94 | 46 |
| 266 | 132 | 2 | 209 | 1 | 2 | 88 | 46 |
| 267 | 132 | 2 | 209 | 1 | 2 | 90 | 42 |
| 268 | 132 | 2 | 104 | 1 | 2 | 84 | 40 |
| 269 | 209 | 3 | 104 | 1 | WT | 112 | 46 |
| 270 | 209 | 3 | 104 | 1 | WT | 112 | 48 |
| 271 | 209 | 3 | 104 | 1 | WT | 111 | 45 |
| 272 | 209 | 3 | 104 | 1 | WT | 111 | 44 |
| 273 | 209 | 3 | 104 | 1 | WT | 118 | 46 |
| 274 | 209 | 3 | 104 | 1 | WT | 111 | 42 |
| 275 | 209 | 3 | 158 | 1 | WT | 122 | 49 |
| 276 | 209 | 3 | 158 | 1 | WT | 111 | 48 |
| 277 | 209 | 3 | 158 | 1 | WT | 120 | 50 |
| 278 | 209 | 3 | 158 | 1 | WT | 118 | 48 |
| 279 | 209 | 3 | 158 | 1 | WT | 133 | 54 |
| 280 | 209 | 3 | 158 | 1 | WT | 123 | 52 |
| 281 | 209 | 3 | 158 | 1 | WT | 116 | 48 |
| 282 | 209 | 3 | 158 | 1 | WT | 123 | 43 |
| 283 | 209 | 3 | 158 | 1 | WT | 119 | 48 |
| 284 | 209 | 3 | 158 | 1 | WT | 118 | 37 |
| 285 | 209 | 3 | 158 | 1 | WT | 112 | 48 |
| 286 | 209 | 3 | 158 | 1 | WT | 104 | 40 |
| 287 | 209 | 3 | 158 | 1 | WT | 106 | 31 |
| 288 | 209 | 3 | 158 | 1 | WT | 104 | 41 |
| 289 | 209 | 3 | 158 | 1 | WT | 101 | 41 |
| 290 | 209 | 3 | 158 | 1 | WT | 102 | 35 |
| 291 | 104 | 3 | 158 | 1 | 1 | 118 | 42 |
| 292 | 104 | 3 | 158 | 1 | 1 | 114 | 49 |
| 293 | 104 | 3 | 158 | 1 | 1 | 120 | 51 |
| 294 | 104 | 3 | 128 | 1 | 1 | 106 | 37 |
| 295 | 104 | 3 | 128 | 1 | 1 | 114 | 48 |
| 296 | 104 | 3 | 128 | 1 | 1 | 117 | 49 |
| 297 | 104 | 3 | 128 | 1 | 1 | 130 | 52 |
| 298 | 158 | 3 | 128 | 1 | 3 | 116 | 45 |
| 299 | 158 | 3 | 128 | 1 | 3 | 109 | 45 |
| 300 | 158 | 3 | 128 | 1 | 3 | 111 | 46 |
| 301 | 158 | 3 | 128 | 1 | 3 | 110 | 46 |
| 302 | 158 | 3 | 128 | 1 | 3 | 114 | 45 |
| 303 | 158 | 3 | 128 | 1 | 3 | 119 | 41 |
| 304 | 158 | 3 | 128 | 1 | 3 | 114 | 52 |
| 305 | 158 | 3 | 128 | 1 | 3 | 116 | 45 |
| 306 | 158 | 3 | 128 | 1 | 3 | 117 | 46 |
| 307 | 158 | 3 | 128 | 1 | 3 | 121 | 47 |
| 308 | 158 | 3 | 128 | 1 | 3 | 115 | 48 |
| 309 | 158 | 3 | 128 | 1 | 3 | 107 | 41 |
| 310 | 158 | 3 | 128 | 1 | 3 | 126 | 56 |
| 311 | 158 | 3 | 133 | 1 | 3 | 117 | 43 |
| 312 | 158 | 3 | 133 | 1 | 3 | 118 | 45 |
| 313 | 158 | 3 | 133 | 1 | 3 | 113 | 44 |
| 314 | 158 | 3 | 133 | 1 | 3 | 107 | 40 |
| 315 | 158 | 3 | 133 | 1 | 3 | 115 | 47 |
| 316 | 158 | 3 | 133 | 1 | 3 | 109 | 42 |
| 317 | 128 | 3 | 133 | 1 | 2 | 119 | 47 |
| 318 | 128 | 3 | 133 | 1 | 2 | 114 | 54 |
| 319 | 128 | 3 | 133 | 1 | 2 | 114 | 45 |
| 320 | 128 | 3 | 133 | 1 | 2 | 114 | 48 |
| 321 | 128 | 3 | 133 | 1 | 2 | 114 | 43 |
| 322 | 128 | 3 | 133 | 1 | 2 | 118 | 46 |
| 323 | 128 | 3 | 133 | 1 | 2 | 115 | 47 |
| 324 | 128 | 3 | 133 | 1 | 2 | 118 | 48 |
| 325 | 128 | 3 | 133 | 1 | 2 | 117 | 49 |
| 326 | 128 | 3 | 133 | 1 | 2 | 115 | 49 |
| 327 | 128 | 3 | 133 | 1 | 2 | 120 | 49 |
| 328 | 128 | 3 | 133 | 1 | 2 | 111 | 39 |
| 329 | 128 | 3 | 133 | 1 | 2 | 114 | 47 |
| 330 | 128 | 3 | 133 | 1 | 2 | 96 | 41 |
| 331 | 128 | 3 | 133 | 1 | 2 | 103 | 40 |
| 332 | 128 | 3 | 133 | 1 | 2 | 108 | 44 |
| 333 | 128 | 3 | 133 | 1 | 2 | 105 | 42 |

Plant and ear heights were measured at maturity for each hybrid plant. Results are summarized in TABLES 17 and 18. Plants were compared to their wildtype counterparts of the same hybrid. Hybrids of inbred 2 by edits 1, 2, and 3 were significantly shorter than non-edited hybrid plants. Hybrids of inbred 3 were not significantly shorter than their non-edited counterparts, which could point to other alleles in inbred 3 that affect plant height.

TABLE 17
Average plant height for edited and wildtype hybrids.

| | Avg plant | | | t- | |
|---|---|---|---|---|---|
| Hybrid | height in | | | sta- | p- |
| (parent 1 × parent 2)_edit | inches | STDEV | N | tistic | value |
| Inbred 2 × Inbred 1_WT | 109.04 | 7.58 | 23 | — | — |
| Inbred 2 × Inbred 1_edit 1 | 101.75 | 6.28 | 16 | −3.17 | 0.0031 |
| Inbred 2 × Inbred 1_edit 2 | 90.20 | 4.49 | 5 | −5.31 | <0.0001 |
| Inbred 2 × Inbred 1_edit 3 | 99.50 | 3.89 | 8 | −3.38 | 0.0021 |
| Inbred 3 × Inbred 1_WT | 113.95 | 7.94 | 22 | — | — |
| Inbred 3 × Inbred 1_edit 1 | 117.00 | 7.28 | 7 | 0.90 | 0.37625 |
| Inbred 3 × Inbred 1_edit 2 | 112.65 | 6.34 | 17 | −0.56 | 0.5822 |
| Inbred 3 × Inbred 1_edit 3 | 114.42 | 4.90 | 19 | 0.22 | 0.82556 |

TABLE 18
Average ear height for edited and wildtype hybrids.

| | Avg ear | | | | |
|---|---|---|---|---|---|
| Hybrid | height in | | | t- | p- |
| (parent 1 × parent 2)_edit | inches | STDEV | N | statistic | value |
| Inbred 2 × Inbred 1_WT | 51.91 | 4.83 | 23 | — | — |
| Inbred 2 × Inbred 1_edit 1 | 47.38 | 4.13 | 16 | −3.06 | 0.00414 |
| Inbred 2 × Inbred 1_edit 2 | 43.80 | 2.68 | 5 | −3.60 | 0.00132 |
| Inbred 2 × Inbred 1_edit 3 | 46.88 | 3.04 | 8 | −2.75 | 0.01021 |
| Inbred 3 × Inbred 1_WT | 44.73 | 5.59 | 22 | — | — |
| Inbred 3 × Inbred 1_edit 1 | 46.86 | 5.40 | 7 | 0.88 | 0.38419 |
| Inbred 3 × Inbred 1_edit 2 | 45.76 | 3.88 | 17 | 0.65 | 0.51822 |
| Inbred 3 × Inbred 1_edit 3 | 45.47 | 3.78 | 19 | 0.49 | 0.62509 |

Concurrently with the hybrid greenhouse generation, homozygous-edited F3 seeds from selfed F2 plants for edits 2 and 3 were planted to create sufficient hybrid seed for field testing. F3 seeds were bulked from five F2 plants of each edit, as described in TABLE 19. Homozygous-edited F3 plants from selfed F2 plants were crossed with plants of inbred 2 to produce hybrid seed. In addition, F3 seeds from edits 1, 2, 3, and 4 were planted and selfed to generate homozygous F4 seed.

TABLE 19
F3 seed inventories planted in hybrid nursery.

| | F2 parent | F2 parent | |
|---|---|---|---|
| F3 seed bulk ID | plant ID | inbred ID | Edit ID |
| F3 seed bulk 1 | 101 | 1 | 1 |
| | 102 | 1 | 1 |
| | 103 | 1 | 1 |
| | 104 | 1 | 1 |
| | 105 | 1 | 1 |
| F3 seed bulk 2 | 128 | 1 | 2 |
| | 129 | 1 | 2 |
| | 130 | 1 | 2 |
| | 131 | 1 | 2 |
| | 132 | 1 | 2 |
| F3 seed bulk 3 | 155 | 1 | 3 |
| | 156 | 1 | 3 |
| | 157 | 1 | 3 |
| | 158 | 1 | 3 |
| | 159 | 1 | 3 |

### Example 6: Hybrid Field Experiment and Related Data

Hybrid seed was produced from F3 homozygous-edited plants (sources described in TABLE 19) of inbred 1 crossed to wildtype plants of inbred 2. Hybrid control seed was also produced from wildtype plants of inbred 1 crossed to wildtype plants of inbred 2 in the same nursery. Wildtype and heterozygous-edited hybrid seed were planted in the field in a randomized complete block design, with 8 entries per hybrid. Plant height for each entry was measured at the R2 growth stage (see TABLE 20), and 10 leaf samples from each hybrid were collected at V10 and analyzed for hormone concentrations (see TABLE 21).

For hormone analysis, the fresh frozen plant leaf tissues were extracted and cleaned up using Waters solid phase extraction MAX cartridge plate. Total auxin (IAA) hormones, and internal standard were analyzed using UPLC coupled with an ABSciex 5500 Mass Spectrometer using the MRM method. The final auxin (IAA) values were calculated based on the calibration curve with ABSciex software OS Multi-Quan. Each IAA calibration curve had a good linear fit with the R2 linear regression >0.99. The 8 technical controls per 96 well plate for IAA were also included and evaluated in analytical process for meeting the standard criterion which is <10% CV.

Final plant height of the heterozygous edited hybrids were about 11 inches shorter on average (over 8 plots; one plant per plot) than their wildtype hybrid counterparts, which was statistically significant at p≤0.05. Further, IAA (auxin)hormone levels were statistically decreased in hybrid plant leaves with edit 3 compared to wildtype and were trending lower for edit 2 although the change was not statistically significant (TABLE 22). As Br2 is an auxin transporter, this reduction in auxin in the leaves could be due to reduced transport from the site of auxin biosynthesis to leaf tissue.

TABLE 20
Hybrid plant height at R2 growth stage in field experiment.

| | Inbred | Inbred | Mean plant | Standard | | |
|---|---|---|---|---|---|---|
| Edit | parent 1 | parent 2 | height at R2 | error | Number of | p- |
| ID | ID | ID | in inches | of mean | plots (N) | value |
| 2 | 1 | 2 | 88.95 | 0.55 | 8 | 0.00 |
| 3 | 1 | 2 | 89.11 | 0.55 | 8 | 0.00 |
| WT | 1 | 2 | 100.07 | 0.55 | 8 | |

TABLE 21
Hormone analysis at V10 growth stage in field experiment.

| Plant | Edit | Hybrid (parent 1 inbred ID × | IAA |
|---|---|---|---|
| ID | ID | parent 2 inbred ID) | (pmol/g) |
| 334 | 2 | 1 × 2 | 552.61 |
| 335 | 2 | 1 × 2 | 80.06 |
| 336 | 2 | 1 × 2 | 868.24* |
| 337 | 2 | 1 × 2 | 0.00 |
| 338 | 2 | 1 × 2 | 98.04 |
| 339 | 2 | 1 × 2 | 242.54 |
| 340 | 2 | 1 × 2 | 74.41 |
| 341 | 2 | 1 × 2 | 88.18 |
| 342 | 2 | 1 × 2 | 120.15 |
| 343 | 2 | 1 × 2 | 0.27 |
| 344 | 3 | 1 × 2 | 95.73 |
| 345 | 3 | 1 × 2 | 88.74 |
| 346 | 3 | 1 × 2 | 129.92 |
| 347 | 3 | 1 × 2 | 135.31 |
| 348 | 3 | 1 × 2 | 77.83 |
| 349 | 3 | 1 × 2 | 96.41 |
| 350 | 3 | 1 × 2 | 104.61 |
| 351 | 3 | 1 × 2 | 124.16 |
| 352 | 3 | 1 × 2 | 90.74 |
| 353 | 3 | 1 × 2 | 87.70 |
| 354 | WT | 1 × 2 | 471.13 |
| 355 | WT | 1 × 2 | 153.73 |
| 356 | WT | 1 × 2 | 85.52 |
| 357 | WT | 1 × 2 | 78.68 |
| 358 | WT | 1 × 2 | 296.68 |
| 359 | WT | 1 × 2 | 0.00 |
| 360 | WT | 1 × 2 | 88.19 |
| 361 | WT | 1 × 2 | 402.28 |
| 362 | WT | 1 × 2 | 83.27 |
| 363 | WT | 1 × 2 | 156.72 |

*statistical outlier omitted from analysis

TABLE 22
Statistics of hormone analysis in field experiment.

| | | | | | Percent | |
|---|---|---|---|---|---|---|
| | Hybrid ID | Average | Standard | | change | |
| Edit | (Parent | IAA | error | | from | p- |
| ID | Inbreds) | (pmon/g) | of mean | N | WT | value |
| 2 | 1 × 2 | 139.58 | 32.26 | 9 | −23.15% | 0.3467 |
| 3 | 1 × 2 | 103.12 | 30.60 | 10 | −43.23% | 0.0726 |
| WT | 1 × 2 | 181.62 | 30.60 | 10 | | |

### Example 7: F4 Controlled Environment Experiment and Related Data

F3 seeds (sources described in Table 19) were planted and selfed to generate homozygous edited F4 seed. The resulting homozygous edited F4 seed was planted in a controlled environment for a destructive sampling experiment. At V4 growth stage, three plants for each edit plus wildtype were sampled at the base of the third leaf. At V6 growth stage, eight plants for each edit plus wildtype were sampled at the following locations: approximately 3 g of tissue from the base of the uppermost expanded leaf, an approximate 1 cm cross-section of the node corresponding to the uppermost expanded leaf, and an approximate 1 cm cross-section of the internode above the sampled node. Sampled tissue was flash-frozen in liquid nitrogen, milled, and aliquoted for both RNA and hormone analyses.

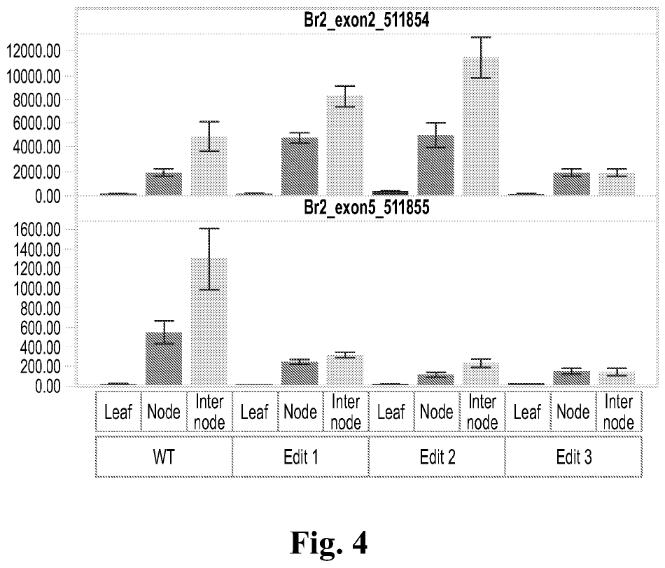

Total RNA was extracted from the V6 samples, and relative mRNA expression levels were quantified as in Example 4. Again, data for each sample was normalized to the Zm·EF1a gene. Assay primers and probes are described in Table 8. Results are summarized in Tables 23 and 24, and FIG. 4. For all samples, the relative expression of Br2 was higher in nodes and internodes than leaves, which is expected based on published reports of Br2 expression. For the exon 2 assay, the relative expression of Br2 was higher in edit 1 and 2 nodes and internodes than wildtype nodes and internodes. These results are similar to the F2 results presented in FIG. 3B. Also similar to F2 results, the relative level of expression was decreased in all edited node and internode samples on the exon 5 assay.

To explain the discrepancy between exon 2 and exon 5 assays, the RNA transcripts were more thoroughly characterized with a quantitative sequencing approach using the V4 samples. To further characterize Br2 transcripts from the edits, a RACE (Rapid Amplification of cDNA Ends) experiment was conducted. Total RNA was extracted from V4 leaves of 3 plants for each edit plus wildtype. Amplicons were generated using the SMARTer® RACE 5′/3′ Kit (Clontech, 634858). In brief, cDNA was synthesized from 250 ng of total RNA as input using modified oligo(dT) primers supplied with the kit, then diluted in 50 μl of Tricine-EDTA buffer. Amplicons were created using 25 cycles of touch down PCR with a gene specific primer located upstream of all edits (SEQ ID NO: 48) and Universal Primer A Mix. A post PCR cleanup was done using a 1:1 ratio of SeqPure beads (biochain) and elution in 15 ul of elution buffer, next products were verified using the fragment analyzer (DNF-474-0500). Final libraries were created using the Nextera DNA Flex Library Prep Kit (Illumina, 20018708). The final amplification and adapter addition were done using Kappa HiFi HotStart ReadyMix (Roche, KK2602). Libraries were sequenced with a NextSeq 500/550 mid output kit v2.5, 300 cycles (Illumina, 20024905).

For data analysis, reads were adaptor trimmed (trim_galore) and then mapped to genomic DNA sequence files (with or without gene edits, as appropriate) using a splice-aware aligner (STAR). Alignments were visualized using Integrative Genomics Viewer javascript (IGV.js). The read alignments are summarized in FIG. 5 showing that RNA transcripts expressed from most of the edited br2 alleles (except for edit 4) tended to trail off or diminish with a truncated overall length after or downstream of the site or region of the edit (inversion and/or deletion) as opposed to wild type.

Wildtype transcript spliced and terminated as expected based on published literature. The following observations were made from this experiment. For Edit 1 (inversion between SP1 and SP2), the transcript ended at the inversion, indicating the inversion could have introduced novel transcription stop sites, although the transcript did include inverted exon 4 before termination. For Edit 2 (inversion between guide SP1 and SP3, with truncation at 3′ end of inversion), the mRNA was transcribed through much of the inversion and dropped off shortly before the non-edited portion of exon 5. For transcripts that included the inverted intron, the intron appeared to be retained and not spliced. For Edit 3 (inversion between guide SP2 and SP3, inserted into cut site of SP1), the transcript included the targeted and inverted fragments in the transcript, and some but not all of the transcripts trailed off downstream of the edit. There was some splicing observed after the edit, but to novel splice sites rather than to the expected splice site before exon 4. For Edit 4 (simple deletions at guides SP1, SP2, and SP3), the transcript appeared to splice normally, although there was some evidence that intron 3 may have been retained in some transcripts. Taken together, these results indicate that the edit-induced inversions in the Br2 gene do create an antisense sequence in the RNA transcript that could potentially hybridize to a corresponding sequence of a wildtype transcript in a heterozygous plant and cause RNA silencing or suppression of one or both transcripts.

TABLE 23
br2 mRNA expression analysis at node
6 of V10 plants, exon 2 assay.

| | | | | | t-statistic | p-value |
|---|---|---|---|---|---|---|
| | | Relative | | | compared | compared |
| Edit | Tissue | expression | STDEV | N | to WT | to WT |
| 1 | Leaf | 160.6 | 125.1 | 8 | 1.32 | 0.2067 |
| 1 | Node | 4764.5 | 1107.4 | 8 | 5.89 | 0.0000 |
| 1 | Internode | 8247.8 | 2469.2 | 8 | 2.25 | 0.0408 |
| 2 | Leaf | 354.3 | 89.7 | 8 | 5.98 | 0.0000 |
| 2 | Node | 4968.2 | 2891.9 | 8 | 2.92 | 0.0113 |
| 2 | Internode | 11433.4 | 4712.3 | 8 | 3.18 | 0.0067 |
| 3 | Leaf | 116.4 | 66.0 | 8 | 0.70 | 0.4928 |
| 3 | Node | 1837.7 | 886.0 | 8 | −0.06 | 0.9568 |
| 3 | Internode | 1828.5 | 834.8 | 8 | −2.47 | 0.0269 |
| WT | Leaf | 89.1 | 87.6 | 8 | — | — |
| WT | Node | 1861.6 | 847.1 | 8 | — | — |
| WT | Internode | 4895.0 | 3407.6 | 8 | — | — |

TABLE 24
br2 mRNA expression analysis at node
6 of V10 plants, exon 5 assay.

| | | | | | t-statistic | p-value |
|---|---|---|---|---|---|---|
| | | Relative | | | compared | compared |
| Edit | Tissue | expression | STDEV | N | to WT | to WT |
| 1 | Leaf | 4.2 | 5.0 | 8 | −2.11 | 0.0535 |
| 1 | Node | 249.9 | 61.5 | 8 | −2.71 | 0.0169 |
| 1 | Internode | 326.3 | 83.3 | 8 | −3.15 | 0.0071 |
| 2 | Leaf | 12.6 | 4.0 | 8 | −0.68 | 0.5098 |
| 2 | Node | 117.7 | 62.9 | 8 | −3.89 | 0.0016 |
| 2 | Internode | 235.0 | 111.7 | 8 | −3.43 | 0.0040 |
| 3 | Leaf | 21.3 | 8.7 | 8 | 0.76 | 0.4594 |
| 3 | Node | 155.2 | 80.1 | 8 | −3.51 | 0.0035 |
| 3 | Internode | 149.3 | 95.1 | 8 | −3.71 | 0.0023 |
| WT | Leaf | 16.5 | 15.7 | 8 | — | — |
| WT | Node | 553.1 | 310.4 | 8 | — | — |
| WT | Internode | 1303.5 | 873.6 | 8 | — | — |

Homozygous edited plants were sampled as above, and hormone analysis was conducted as in example 6. Results are summarized in TABLE 25. IAA levels in leaves and internodes of edited plants were reduced in some cases but were not statistically different from wildtype. However, IAA was significantly reduced in V6 nodes for all edits compared to wildtype. These results may differ from the field hormone data presented in Example 6 because the sample timings were different. The controlled environment samples were taken at V6 stage when internodes are actively elongating, while field samples were taken at V10 stage closer to maturity. Hormone levels change dramatically at different timepoints in different tissues, so it is expected that IAA levels with V6 and V10 sample timings would be different.

Br2 is an auxin transporter, thought to transport auxin from the node to internode through vascular bundles, and promote elongation in internodes. The significant reduction in auxin at the sixth node of edited plants is similar to previous results, where free IAA levels in nodes were significantly reduced in a natural br2 mutant (see, e.g., FIG. 4B of Knöller, A. S. et al., Journal of Expt. Biology, 61(13):3689 (2010), the contents and disclosure of which are incorporated herein by reference).

The results presented in this example show that these brachytic-2 edits in maize reduce Br2 gene expression and auxin accumulation in key plant tissues in a homozygous state and reduce plant height in both a heterozygous and homozygous state.

TABLE 25
Hormone analysis at V6 growth stage of
F4 plants in controlled environment.

| | | | | | Percent | |
|---|---|---|---|---|---|---|
| | | | | | increase | |
| | | Average | Standard | | or | |
| Edit | Sample | IAA | error | | reduction | p- |
| ID | location | (pmon/g) | of mean | N | from WT | value |
| 1 | Leaf | 71.95 | 6.26 | 8 | −7.77 | 0.4983 |
| 1 | Node | 43.82 | 2.41 | 8 | −25.51 | 0.0001 |
| 1 | Internode | 50.24 | 44.08 | 8 | −52.59 | 0.3776 |
| 2 | Leaf | 76.26 | 6.26 | 8 | −2.24 | 0.8447 |
| 2 | Node | 45.13 | 2.25 | 8 | −23.28 | 0.0001 |
| 2 | Internode | 160.28 | 47.12 | 8 | 51.26 | 0.4058 |
| 3 | Leaf | 72.02 | 6.26 | 8 | −7.68 | 0.5033 |
| 3 | Node | 45.82 | 2.41 | 8 | −22.09 | 0.0004 |
| 3 | Internode | 79.51 | 44.08 | 8 | −24.97 | 0.6739 |
| 4 | Leaf | 75.44 | 6.26 | 8 | −3.30 | 0.7733 |
| 4 | Node | 42.74 | 2.25 | 8 | −27.34 | 0.0001 |
| 4 | Internode | 118.41 | 44.08 | 8 | 11.75 | 0.8429 |
| WT | Leaf | 78.01 | 6.26 | 8 | — | — |
| WT | Node | 58.82 | 2.25 | 8 | — | — |
| WT | Internode | 105.96 | 44.08 | 8 | — | — |

### Example 3: Hybrid Field Experiment for Corn Plants of Edit ID 1

Corn plants of F3 homozygous seeds of Edit ID I (source described in Table 19) were crossed with wild-type plants of Inbred 2 or 3. Hybrid control seeds were also produced from wild-type plants of Inbred 1 crossed with wild-type plants of Inbred 2 or 3 in the same nursery. Insect damage in the hybrid nursery prevented all combinations from being planted.

Wild-type and heterozygous edited hybrid corn seeds were planted in the field in a randomized complete block design with 3 to 9 entries per hybrid at 3 or 4 locations. Plant height was measured from three plants at maturity (R2 growth stage) for each entry, and statistical comparisons were done by germplasm. It was noted that overall growing conditions during this field trial season were not ideal, especially for Location 2 where all plant heights were shorter than expected. However, comparisons between entries within each location are made to determine if edited alleles are significantly shorter than their wild-type counterparts.

TABLE 26
Hybrid plant height at R2 growth stage in field experiment

| | Inbred | Inbred | | Mean plant | Standard | | |
|---|---|---|---|---|---|---|---|
| Edit | parent 1 | parent 2 | | height at R2 | error of | Number of | |
| ID | ID | ID | Location | in inches | mean | plots (N) | p-value |
| 1 | 1 | 2 | 1 | 93.31 | 0.89 | 9 | 3.0169E−06 |
| 1 | 1 | 2 | 2 | 57.96 | 1.08 | 9 | 0.9771651 |
| 1 | 1 | 2 | 3 | 65.00 | 1.22 | 9 | 4.8069E−09 |
| 1 | 1 | 2 | 4 | 85.96 | 0.87 | 9 | 0.05132365 |
| WT | 1 | 2 | 1 | 99.39 | 0.89 | 9 | |
| WT | 1 | 2 | 2 | 58.01 | 1.08 | 9 | |
| WT | 1 | 2 | 3 | 75.72 | 1.22 | 9 | |
| WT | 1 | 2 | 4 | 88.36 | 0.87 | 9 | |
| 1 | 1 | 3 | 1 | 87.09 | 1.29 | 3 | 3.8214E−07 |
| 1 | 1 | 3 | 2 | 60.89 | 1.59 | 3 | 0.9116007 |
| 1 | 1 | 3 | 4 | 85.34 | 1.46 | 3 | 0.00068597 |
| WT | 1 | 3 | 1 | 96.77 | 1.30 | 3 | |
| WT | 1 | 3 | 2 | 61.15 | 1.61 | 3 | |
| WT | 1 | 3 | 4 | 92.52 | 1.47 | 3 | |

In summary, hybrid edited plants were significantly shorter than their wildtype comparators at three of the four locations. Height of edited plants was reduced by up to 10 inches, or by 14%, in comparison with the corresponding wild-type plants. This plant height reduction was s greater than would be expected from a heterozygous recessive allele.

1. A modified corn plant, or plant part thereof, comprising a mutant allele of an endogenous Brachytic2 (br2) locus, wherein the mutant allele comprises a DNA segment inserted into the endogenous br2 locus, wherein the DNA segment encodes an antisense RNA sequence that is at least 85% complementary to at least 20 consecutive nucleotides of one or more of SEQ ID NOs: 1 and 50, and wherein the mutant allele of the endogenous br2 locus produces a RNA transcript comprising the antisense RNA sequence.

2. The modified corn plant, or plant part thereof, of claim 1, wherein the mutant allele of the endogenous br2 locus suppresses the expression and/or disrupts the function of a wild-type allele of the endogenous br2 locus.

3. The modified corn plant, or plant thereof, of claim 1, wherein the mutant allele product of the endogenous br2 locus disrupts the function of a wild-type allele product of the endogenous br2 locus.

4. The modified corn plant, or plant part thereof, of claim 1, wherein the RNA transcript further comprises one or more sequence elements of the endogenous br2 locus selected from the group consisting of 5′UTR, first exon, first intron, second exon, second intron, third exon, third intron, fourth exon, fourth intron, fifth exon, 3′ UTR, and any portion thereof.

5. The modified corn plant, or plant part thereof, of claim 1, wherein the DNA segment comprises a nucleotide sequence originating from the endogenous br2 locus.

6. The modified corn plant, or plant part thereof, of claim 5, wherein the DNA segment corresponds to an inverted genomic fragment of the endogenous br2 locus.

7. The modified corn plant, or plant part thereof, of claim 1, wherein at least a portion of the antisense RNA sequence is at least 90% complementary to a corresponding endogenous sequence of the RNA transcript.

8. The modified corn plant, or plant part thereof, of claim 7, wherein the corresponding endogenous sequence of the RNA transcript is at least 90%, identical to at least 20 consecutive nucleotides of one or more of SEQ ID NOs: 1 and 50.

9. The modified corn plant, or plant part thereof, of claim 7, wherein the antisense RNA sequence hybridizes to the corresponding endogenous sequence of the RNA transcript.

10. The modified corn plant, or plant part thereof, of claim 7, wherein the DNA segment is inserted near or adjacent to a corresponding endogenous DNA segment of the endogenous br2 locus.

11. The modified corn plant, or plant part thereof, of claim 10, wherein the antisense RNA sequence encoded by the inserted DNA segment hybridizes to a corresponding endogenous sequence of the RNA transcript encoded by the corresponding endogenous DNA segment.

12. The modified corn plant, or plant part thereof, of claim 10, wherein the antisense RNA sequence forms a stem-loop structure with the corresponding endogenous sequence of the RNA transcript.

13. The modified corn plant, or plant part thereof, of claim 10, wherein the inserted DNA segment and the corresponding endogenous DNA segment of the mutant allele are separated by an intervening DNA sequence.

14. The modified corn plant, or plant part thereof, of claim 13, wherein the intervening DNA sequence has a length of at least 2 consecutive nucleotides.

15. The modified corn plant, or plant part thereof, of claim 13, wherein the DNA segment and the corresponding endogenous DNA segment are separated by an intervening sequence of at most 4,000 consecutive nucleotides.

16. The modified corn plant, or plant part thereof, of claim 13, wherein the intervening DNA sequence encodes an intervening RNA sequence between the antisense RNA sequence and the corresponding endogenous sequence of the RNA transcript.

17. The modified corn plant, or plant part thereof, of claim 16, wherein the RNA transcript forms a stem-loop structure with the intervening RNA sequence forming the loop portion of the stem-loop structure.

18. The modified corn plant, or plant part thereof, of claim 17, wherein (a) the stem-loop secondary structure contains a near-perfect-complement stem with mismatches: or (b) the stem-loop secondary structure contains a perfect-complement stem with no mismatch.

19. (canceled)

20. The modified corn plant, or plant part thereof, of claim 13, wherein:
(a) the intervening DNA sequence comprises a native sequence of the endogenous br2 locus;
(b) the intervening DNA sequence comprises an exogenous sequence inserted into the endogenous br2 locus;
(c) the intervening sequence contains an intron sequence; or
(d) the intervening sequence does not contain an intron sequence.

21. (canceled)

22. (canceled)

23. (canceled)

24. The modified corn plant, or plant part thereof, of claim 10, wherein (a) the inserted DNA segment is located upstream of the corresponding endogenous DNA segment; or (b) the inserted DNA segment is located downstream of the corresponding endogenous DNA segment.

25. (canceled)

26. The modified corn plant, or plant part thereof, of claim 7, wherein the DNA segment is inserted within a region selected from the group consisting of 5′ untranslated region (UTR), first exon, first intron, second exon, second intron, third exon, third intron, fourth exon, fourth intron, fifth exon, and 3′ UTR of the endogenous br2 locus, and a combination thereof.

27. The modified corn plant, or plant part thereof, of claim 7, wherein the DNA segment is inserted at a genomic site recognized by a targeted editing technique to create a double-stranded break (DSB).

28. The modified corn plant, or plant part thereof, of claim 7, wherein the mutant allele further comprises a deletion of at least one portion of the endogenous br2 locus.

29. The modified corn plant, or plant part thereof, of claim 7, wherein:
(a) the sense strand of the DNA segment comprises a sequence at least 80% complementary to an exon sequence of the endogenous br2 locus;
(b) the sense strand of the DNA segment comprises a sequence at least 80% complementary to an untranslated region (UTR) sequence of the endogenous br2 locus;
(c) the sense strand of the DNA segment comprises a sequence at least 80% complementary to an exon sequence and an intron sequence of the endogeneous br2 locus, the exon sequence and the intron sequence being Contiguous within the endogenous locus.

30. (canceled)

31. (canceled)

32. The modified corn plant, or plant part thereof, of claim 7, wherein the DNA segment comprises a sequence having at least 80% identity to one or more of SEQ ID Nos: 1 and 50.

33. The modified corn plant, or plant part thereof, of claim 1, wherein the corn plant is homozygous for the mutant allele at the endogenous br2 locus.

34. The modified corn plant, or plant part thereof, of claim 1, wherein the corn plant is heterozygous for the mutant allele at the endogenous br2 locus.

35. The modified corn plant, or plant part thereof, of claim 1, wherein the DNA segment has a length of at least 15 nucleotides.

36. The modified corn plant, or plant part thereof, of claim 1, wherein the DNA segment has a length of at most 1000 nucleotides.

37. The modified corn plant, or plant part thereof, of claim 1, wherein the modified corn plant has a shorter plant height and/or improved lodging resistance relative to an unmodified control plant.

38. The modified corn plant, or plant part thereof, of claim 1, wherein the modified corn plant exhibits an at least 2.5% reduction in plant height at maturity relative to an unmodified control plant.

39. The modified corn plant, or plant part thereof, of claim 38, wherein the plant height reduction is between 5% and 40%.

40. The modified corn plant, or plant part thereof, of claim 1, wherein the modified corn plant does not have any significant off-types in at least one female organ or ear.

41. The modified corn plant, or plant part thereof, of claim 1, wherein the modified corn plant exhibits essentially no reproductive abnormality.

42. A method for producing a mutant allele of an endogenous Brachytic2 (br2) locus, the method comprising:
(a) generating a first double-stranded break (DSB) in the endogenous br2 locus in a corn cell using a targeted editing technique;
(b) inserting at the first DSB a DNA segment using a targeted editing technique, wherein the DNA segment encodes an antisense RNA sequence that is at least 85% complementary to at least 20 consecutive nucleotides of one or more of SEQ ID NOs: 1 and 50, and wherein the mutant allele of the endogenous br2 locus produces a RNA transcript comprising the antisense RNA sequence.

43. The method of claim 42, wherein the targeted editing technique comprises the use of at least one site-specific nuclease.

44. The method of claim 43, wherein the at least one site-specific nuclease is selected from the group consisting of a zinc-finger nuclease, a meganuclease, an RNA-guided nuclease, a TALE-nuclease, a recombinase, a transposase, and any combination thereof.

45. (canceled)

46. The method of claim 42, wherein the DNA segment originates from the endogenous br2 locus.

47. The method of claim 42, wherein the DNA segment is provided in a donor template.

48. The method of claim 42, wherein the method further comprises regenerating or developing a corn plant from the corn cell.

49. The method of claim 42, wherein the mutant allele of the endogenous br2 locus is capable of suppressing the expression of a wild-type allele of the endogenous br2 locus.

50.-70. (canceled)

71. A method for generating a corn plant comprising:
(a) fertilizing at least one female corn plant with pollen from a male corn plant, wherein said female corn plant comprises a mutant allele of an endogenous Brachytic2 (br2) locus, wherein the mutant allele comprises a DNA segment inserted into the endogenous br2 locus, wherein the DNA segment encodes an antisense RNA sequence that is at least 85% complementary to at least 20 consecutive nucleotides of one or more of SEQ ID NOs: 1 and 50, and wherein the mutant allele of the endogenous br2 locus produces a RNA transcript comprising the antisense RNA sequence; and
(b) obtaining at least one seed produced by said fertilizing of step (a).

72. The method of claim 71, wherein said method further comprises (c) growing said at least one seed obtained in step (b) to generate at least one progeny corn plant comprising said mutant allele.

73.-128. (canceled)
